(19) United States
(12) Patent Application Publication
(10) Pub. No.: US 2022/0244254 A1
(43) Pub. Date: Aug. 4, 2022

(54) Screening Methods and Assays for Use with Transmembrane Proteins, in Particular with GPCRs

(71) Applicant: CONFO THERAPEUTICS N.V., Zwijnaarde (BE)

(72) Inventors: Christel Menet, Brussel (BE); Lies Dekeyzer, Knesselare (BE); Murielle Martini, Blaton (BE); Kamila Skieterska, Gent (BE)

(21) Appl. No.: 17/607,333

(22) PCT Filed: Apr. 28, 2020
(86) PCT No.: PCT/EP2020/061803

§ 371 Date: Oct. 28, 2021

Related U.S. Application Data
(60) Provisional application No. 62/840,091, filed on Apr. 29, 2019.
(60) Provisional application No. 62/840,092, filed on Apr. 29, 2019.
(60) Provisional application No. 62/840,094, filed on Apr. 29, 2019.
(60) Provisional application No. 62/863,544, filed on Jun. 19, 2019.
(60) Provisional application No. 62/934,136, filed on Nov. 12, 2019.
(60) Provisional application No. 62/934,181, filed on Nov. 12, 2019.
(60) Provisional application No. 62/934,133, filed on Nov. 12, 2019.

(51) Int. Cl. G01N 33/566; C07K 16/28; C07K 14/72

(52) U.S. Cl. CPC G01N 33/566; C07K 16/28; C07K 14/723; C07K 2317/569; C07K 2317/567; C07K 2319/74; G01N 2333/726; G01N 2500/04; C07K 2317/31; C07K 2319/03

(57) ABSTRACT

The invention provides methods and arrangements for screening membrane proteins. The arrangement comprises a first fusion protein comprising a membrane protein present in a boundary layer such as a wall of a cell, liposome or vesicle fused to one member of a binding pair and a second fusion protein comprising an intracellular ligand of said membrane protein fused to another member of the binding pair, in which the binding pair is capable of generating a detectable signal.

### CROSS-REFERENCE TO RELATED APPLICATIONS

This application is a national phase entry under 35 U.S.C. § 371 of International Patent Application PCT/EP2020/061803, filed Apr. 28, 2020, designating the United States of America and published in English as International Patent Publication WO 2020/221768 on Nov. 5, 2020, which claims the benefit under Article 8 of the Patent Cooperation Treaty to U.S. Provisional Patent Application Ser. No. 62/840,091, filed Apr. 29, 2019, U.S. Provisional Patent Application Ser. No. 62/840,092, filed Apr. 29, 2019, U.S. Provisional Patent Application Ser. No. 62/840,094, filed Apr. 29, 2019, U.S. Provisional Patent Application Ser. No. 62/863,544, filed Jun. 19, 2019, U.S. Provisional Patent Application Ser. No. 62/934,136, filed Nov. 12, 2019, U.S. Provisional Patent Application Ser. No. 62/934,181, filed Nov. 12, 2019, and U.S. Provisional Patent Application Ser. No. 62/934,133, filed Nov. 12, 2019, the entireties of which are hereby incorporated by reference.

The present invention relates to methods and tools that can be used for assays, for screening and for drug discovery and development efforts.

In particular, the present invention relates to methods and tools that can be used in screening and assay techniques that involve the use of membrane proteins (i.e. as targets to be screened, for example to discover candidate compounds acting against said target) and in efforts to discover, generate, optimize and/or develop therapeutic, prophylactic and diagnostic agents that are directed against (i.e. that have specificity for) membrane proteins. The present invention further relates to methods of making the tools that can be used in the screening and assay techniques.

With advantage, the methods and tools of the invention can be used in screening and assay techniques that involve the use of membrane proteins that can take on/exist in multiple conformations (for example and without limitation, an active and an inactive conformation) and in efforts to discover, generate, optimize and/or develop therapeutic, prophylactic and diagnostic agents that are directed against such membrane proteins. Such membrane proteins include, but are not limited to, transmembrane proteins such as GPCRs and other cell-surface receptors.

In one particularly preferred but non-limiting aspect, the methods and tools of the invention can be used in screening and assay techniques that involve the use of membrane proteins that can undergo a conformational change (again, for example and without limitation, from an inactive conformation to an active conformation) in response to a ligand binding to said protein, and in efforts to discover, generate, optimize and/or develop therapeutic, prophylactic and diagnostic agents that are directed against such membrane proteins. Again, such membrane proteins can be cell-surface receptors such as GPCRs.

The invention generally provides methods which can be used for performing an assay (i.e. on a given compound or ligand) or for screening purposes (i.e. for screening a group, series or library of compounds or ligands to identify “hits” against the target). The invention also provides an arrangement which can be used in said methods, i.e. as a system or set-up for performing said assay or screening. Said arrangements comprise the elements described herein. Said elements can also be provided or established as a kit of parts, and such a kit of parts forms a further aspect of the invention. The invention also provides methods of identifying and creating the components of such arrangements as well as assembling such arrangements.

The methods and arrangements described herein can generally be used for testing a (known) compound or ligand for one or more of its properties (i.e. those properties that can be determined using the methods described herein) and/or for identifying a compound or ligand that has such desired property or properties (i.e. from a group, series or library of compounds or ligands). These compounds or ligands can be any desired and/or suitable compound or ligand, including but not limited to small molecules, small peptides, biological molecules or other chemical entities, and examples of such compounds will be clear to the skilled person based on the further disclosure herein. Also, the compounds that are identified using the methods of the invention (i.e. the “hits” from such screening) can be used as a starting point for further drug discovery and development efforts (e.g. using well-known techniques of so-called “hits-to-leads” chemistry), and such further efforts can also involve the use of the methods of the invention (e.g. as a functional assay or an assay used for quality control purposes).

The compounds that are identified using the methods and techniques of the invention (i.e. as “hits”), and any compounds that are generated or developed using such hits as a starting point, are also collectively referred to as herein “compounds of the invention” and form further aspects of the invention. It will be clear to the skilled person that such compounds may for example be so-called “hits”, “leads”, “development candidates”, “pre-clinical compounds”, “clinical candidates” or commercial compounds or products, depending on their stage of development and on the specific terminology that is used by the company or entity that is developing and/or commercializing them.

With advantage, compared to conventional radioligand assays or functional assays, the methods and assays of the invention do not require the use of a labeled antagonist (e.g. fluorescently labelled or radiolabeled) and thus can also be applied to membrane proteins for which no antagonists are available or known. Also, as further described herein, the methods and assays of the invention may allow allosteric agonists (both positive and negative), antagonists and/or inverse agonists to be identified and/or characterized (depending on the specific target and assay used).

Oher features, aspects, embodiments, uses and advantages of the present invention will become clear from the further description herein.

Membrane proteins (such as cell-surface receptors including GPCRs) and assay and screening techniques for membrane proteins are well-known in the art. It is estimated that over half of all modern medicinal drugs are targeted towards membrane proteins, with roughly a third of all modern medicinal drugs targeting GPCRs. Reference is made to the standard handbooks as well as the further prior art cited herein.

As is well-known from the field of protein dynamics, most proteins are not static objects whose function is determined solely by their primary, secondary, tertiary and—when the protein is comprised of two or more polypeptide chains—quaternary structures, but are often flexible structures that can undergo transitions (also referred to as “conformational changes”) between different conformational states, such that the protein may exist in an equilibrium between these different states. Some of these states may be functional and/or active, while others may be a basal state (which may or may not exhibit some level of constitutive activity), be an essentially inactive state and/or be a less active state compared to more functional or active states. Also, the geometry of the different epitopes, binding sites (including ligand binding sites) and/or catalytic sites that may exist in or on the protein may differ between these different conformations, for example such that in some of the conformational states, a binding site may not be available/accessible for ligand binding and/or such that the affinity for the interaction between the binding site and the relevant ligand(s) is reduced compared to a more active conformational state.

It is also known that for some protein/ligand combinations, binding of a ligand to the protein may change the conformation (for example from an inactive/less active conformation into an active/more active conformation) and/or shift the equilibrium from an inactive/less active conformation towards an active/more active conformation. It is also possible that binding of a ligand to one binding site of a protein may make another binding site on the protein more accessible for its relevant ligand(s) and/or may lead to an increase in the affinity of said other binding site for said ligand(s), and/or shift the equilibrium from a conformation in which said other binding site has less affinity for said ligand(s) towards a conformation in which said other binding site has better affinity for said ligand(s). For example, for some transmembrane proteins such as GPCRs, binding of an extracellular ligand to an extracellular binding site on the protein may increase the affinity of an intracellular binding site for an intracellular ligand (for example, increase the affinity for the interaction between the G-protein and the G-protein binding site on the GPCR), or visa versa. This change in binding affinity for an intercellular ligand following binding of an extracellular ligand, and the subsequent binding of an intracellular ligand to an intracellular binding site, may be part of the way in which the protein transducts an extracellular signal.

Generally, as further described herein, it can be said that for receptor proteins that can undergo a conformation change, an “agonist” of the receptor will shift the conformational equilibrium from an inactive state (or one or more less active states) towards an active state (or one or more states that are more active), whereas an “inverse agonist” of the receptor will do the inverse.

It is also possible that a protein forms a complex with two ligands that bind to two different binding sites on the protein, and that the interaction between the protein and each of the ligands is stabilized by the binding of the other ligand (in other words, that said complex is stabilized by the binding of both ligands). Again, in this case, binding of one or both of the ligands may also shift the conformational equilibrium of the protein towards (the formation and/or stabilization of) this complex. Reference is for example made to WO2012/007593 cited below.

Given that the perceived “overall” state of such a protein is to a large extent governed by the (statistical) distribution of the protein over its various possible conformations, and thus by the equilibrium that exists between these conformational states, it should be understood that when, in the present description or claims, a protein is said to undergo a conformational change into a certain conformation (i.e. from one or more other conformations), this will include a mechanism or situation where the conformational equilibrium of the protein is shifted towards said conformation (i.e. under the specific conditions used, such as the conditions used for screening or the relevant assay). Similarly, when a ligand is said to elicit a conformational change of a protein into a certain conformation (i.e. from one or more other conformations), this includes a mechanism or situation where the binding of the ligand shifts the conformational equilibrium of the protein towards said conformation (i.e. under the specific conditions used, such as the conditions used for screening or the relevant assay).

However, it should also be noted that, although any one of the mechanisms described herein (or any combination thereof) may at any given time be involved in the practice of the invention (also depending on, for example, the specific protein and/or ligand(s) to which the invention is applied), the invention is its broadest sense is not limited to any specific mechanism, explanation or hypothesis as long as the application of the invention to a specific target or protein results in the technical effect(s) outlined herein.

One of the challenges of screening for compounds that are directed against membrane proteins that exist in multiple conformations is that the correct conformation of the protein may be lost if the protein is expressed or used in isolation from its native environment (if it is even feasible or possible to express the protein and to ensure its proper folding outside of its cellular environment). Also, it may be challenging to ensure that the protein is in its desired conformation (often, a functional conformation such as its active conformation) under the conditions that are used for screening. There may also be a need for, or an advantage in achieving, a shift in the conformational equilibrium of the protein towards a conformational state that is more suitable for screening or assay purposes (such as an active state or a state in which the relevant binding site is more accessible for, and/or has a geometry that is better for, assay or screening purposes). As further described herein, such a conformation is also referred to as a “druggable” conformation, and according to preferred aspects of the invention, means are applied (as further described herein) to ensure that the protein is in such a druggable conformation and/or to ensure that the conformational equilibrium of the protein is shifted towards a more druggable conformation when the methods of the invention are performed.

For example, WO2012/007593, WO2012/007594, WO 2012/175643, WO 2014/118297, WO2014/122183 and WO 2014/118297 are directed towards protein binding domains that can be used to stabilize a particular conformational state of a GPCR for the purposes of determining its structure and for drug screening and discovery purposes. In these references, VHH domains are used that can stabilize the GPCR in a desired conformation, and in particular a (more) druggable conformation, such as a functional state and/or active state, for example in the conformation that arises when an activating ligand (agonist) binds to the extracellular side of the GPCR so as to allow the GPCR to activate heterotrimeric G proteins. Reference is for example also made to Pardon et al., Angew Chem Int Ed Engl. 2018, 57(19):5292-5295; Che et al., Cell. 2018, 172(1-2):55-67; Manglik et al., Annu Rev Pharmacol Toxicol. 2017; 57:19-37; Pardon et al., Nat Protoc. 2014, 674-93; Kruse et al., Nature. 2013, 504(7478); Steyaert and Kobilka, Curr Opin Struct Biol. 2011, 567-72; and Rasmussen et al., Nature. 2011, 469(7329): 175-180 and the further references cited therein. VHH domains that can be used to stabilize a desired conformation of a membrane protein such as a GPCR are also referred to herein as ConfoBodies [Confobody™ is a registered trademark of Confo Therapeutics, Ghent, Belgium].

Some specific, but non-limiting examples of ConfoBodies that can bind to an intracellular epitope of a GPCR and that can be used to stabilize a GPCR in a desired conformation (and that can also be used in the present invention) are the VHH called CA2764, CA3431, CA3413, CA2780, CA2765, CA2761, CA3475, CA2770, CA3472, CA3420, CA3433, CA3434, CA3484, CA2760, CA2773, CA3477, CA2774, CA2768, CA3424, CA2767, CA2786, CA3422, CA2763, CA2772, CA2771, CA2769, CA2782, CA2783 and CA2784 (see for example WO 2012/007593, Tables 1 and 2 and SEQ ID NO's: 1 to 29); the VHH called CA5669, Nb9-1, Nb9-8, XA8633 and CA4910 (see for example WO 2014/118297, Tables 1 and 2 and SEQ ID NO's: 15, 16, 17, 19 and 20); the VHH called Nb9-11, Nb9-7, Nb9-7, Nb9-22, Nb9-17, Nb9-24, Nb9-9, Nb9-14, Nb9-2, Nb9-20, Nb_C3, NbH-4, Nb-E1, Nb_A2, Nb_B4, Nb_D3, Nb_D1 and Nb_H1 (see for example WO 2014/122183, Tables 1 and 2 and SEQ ID NOs: 1-19); and the VHH called XA8639, XA8635, XA8727 and XA9644 (see for example WO 2015/121092, Tables 2 and 3 and SEQ ID NOs: 2 to 6 and 74).

Some specific, but non-limiting examples of VHH that can bind to a G-protein are CA4435, CA4433, CA4436, CA4437, CA4440 and CA4441 (see for example WO 2012/175643007593, Tables 2 and 3 and SEQ ID NO's: 1 to 6).

As further described herein, the present invention generally provides improved screening methods and assay techniques that can be used to discover and develop (e.g. to identify, generate, test and optimize) compounds that are directed towards membrane proteins (i.e. that have specificity for one or more membrane proteins and/or that are intended to target one or more membrane proteins, e.g. for therapeutic, prophylactic and/or diagnostic purposes). Preferably, such compounds will be specific for one particular membrane protein compared to other (closely related) membrane proteins (i.e. will be selective for one particular membrane protein).

The compounds identified and/or developed using the methods of the invention can be used to modulate (as defined herein) the membrane protein, its signaling and/or the biological functions, pathways and/or mechanisms in which said membrane protein or its signaling is involved. For example, the invention can be used to discover and develop compounds that are agonists, antagonists, inverse agonists, inhibitors or modulators (such as allosteric modulators, both positive and negative) of said membrane protein and/or of the signaling, the pathway and/or the physiological and/or biological mechanisms in which the membrane protein is involved.

The invention can be used to discover and develop compounds that are directed towards membrane proteins that, in their native environment, are integral membrane proteins or peripheral membrane proteins. The invention can be in particular be used to discover and develop compounds that are directed towards transmembrane proteins, as further described herein. In one specific but non-limiting aspect, the compounds that are discovered and/or developed using the invention will be directed towards a receptor, and in particular a cell-surface receptor.

As further described herein, the transmembrane protein may in particular be a membrane protein with multiple passes through the membrane, such as a 7TM or GPCR. [In this respect, it should be noted that generally, within the field, the terms “7TM receptor” and “7TM” are often used interchangeably with “GPCR”, although according to the IUPHAR database, there are some 7TM receptors that do not signal through G proteins. For the purposes of the present description and claims, the terms “GPCR” and “7TM” are used interchangeably herein to include all transmembrane proteins—and in particular transmembrane receptors—with 7-transmembrane domains, irrespective of their intracellular signaling cascade or signal transduction mechanism, although it should be understood that throughout the description and claims, 7TMs that signal through G-proteins are a preferred aspect of the invention].

Usually, the compounds that are discovered and/or developed using the invention will be directed towards a membrane protein that, when it is in its native environment, is expressed on and/or exposed on the surface of a cell, and in particular towards a membrane protein that is expressed by or on a cell that is present in the body of a subject that is to be treated with a compound that has been discovered or developed using the methods and techniques of the invention.

The invention can be used to discover and/or develop any kind of compound that is suitable for its intended use, which will often be a use as a therapeutic, diagnostic or prophylactic agent. As such, these compounds may be small molecules, peptides, biological molecules or other chemical entities. Examples of suitable biological molecules may for example include antibodies and antibody fragments (such as Fabs, VH, VL and VHH domains) and compounds based on antibody fragments (such as ScFvs and diabodies and other compounds or constructs comprising one or more VH, VL and/or VHH domains), compounds based on other protein scaffolds such Alphabodies™ and scaffolds based on avimers, PDZ domains, protein A domains (such as Affibodies™), ankyrin repeats (such as DARPins™), fibronectin (such as Adnectins™) and lipocalins (such as Anticalins™) as well as binding moieties based on DNA or RNA including but not limited to DNA or RNA aptamers. Reference is made to the further description herein, as well as for example to Simeon and Chen, Protein Cell 2018, 9(1): 3-14, Binz et al, Nat. Biotech 2005, Vol 23: 1257 and Ulrich et al., Comb Chem High Throughput Screen 2006 9(8):619-32.

The methods and techniques of the invention can for example be used to screen libraries of such compounds in order to identify one or more “hits” that are specific for the membrane protein (and in particular for a desired conformation of the membrane protein and/or that are capable of inducing a desired conformation of the membrane protein, such as a ligand-bound and in particular agonist-bound conformation) and/or as an assay that is used as part of a strategy to improve the affinity and/or potency of compounds that are directed against a membrane protein and/or to otherwise improve (the pharmacological and/or other properties of) such a compound (for example, in the case of a small molecule, as part of a “hits-to-leads” campaign).

The methods and techniques of the invention can also be used for the purposes of so-called “fragment-based drug discovery” or “FBDD” (also known as “fragment-based lead discovery” or “FBLD”). Reference is for example made to Lamoree and Hubbard, Essays in Biochemistry (2017) 61, 453-464, and standard handbooks such as Jahnke and Erlanson, “Fragment-based approaches in drug discovery”, 2006; Zartler and Shapiro, “Fragment-based drug discovery: a practical approach”, 2008; and Kuo “Fragment based drug design: tools, practical approaches, and examples”, 2011.

The present invention will be described herein with respect to particular embodiments and with reference to certain non-limiting examples and figures. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Unless otherwise defined herein, scientific and technical terms and phrases used in connection with the present invention shall have the meanings that are commonly understood by those of ordinary skill in the art. Generally, nomenclatures used in connection with, and techniques of molecular and cellular biology, structural biology, biophysics, pharmacology, genetics and protein and nucleic acid chemistry described herein are those well-known and commonly used in the art. Singleton, et al., Dictionary of Microbiology and Molecular Biology, 2D ED., John Wiley and Sons, New York (1994), and Hale & Marham, The Harper Collins Dictionary of Biology, Harper Perennial, NY (1991) provide one of skill with general dictionaries of many of the terms used in this disclosure. The methods and techniques of the present invention are generally performed according to conventional methods well known in the art and as described in various general and more specific references that are cited and discussed throughout the present specification unless otherwise indicated. See, for example, Sambrook et al. Molecular Cloning: A Laboratory Manual, 3th ed., Cold Spring Harbor Laboratory Press, Cold Spring Harbor, N.Y. (2001); Ausubel et al., Current Protocols in Molecular Biology, Greene Publishing Associates (1992, and Supplements to 2002); up, Biomolecular crystallography: principles, Practice and Applications to Structural Biology, 1st edition, Garland Science, Taylor & Francis Group, LLC, an informa Business, N.Y. (2009); Limbird, Cell Surface Receptors, 3d ed., Springer (2004).

As used herein, the terms “polypeptide”, “protein”, “peptide” are used interchangeably herein, and refer to a polymeric form of amino acids of any length, which can include coded and non-coded amino acids, chemically or biochemically modified or derivatized amino acids, and polypeptides having modified peptide backbones. Throughout the application, the standard one letter notation of amino acids will be used. Typically, the term “amino acid” will refer to “proteinogenic amino acid”, i.e. those amino acids that are naturally present in proteins. Most particularly, the amino acids are in the L isomeric form, but D amino acids are also envisaged.

As used herein, the terms “nucleic acid molecule”, “polynucleotide”, “polynucleic acid”, “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

Any of the peptides, polypeptides, nucleic acids, compound, etc., disclosed herein may be “isolated” or “purified”. “Isolated” is used herein to indicate that the material referred to is (i) separated from one or more substances with which it exists in nature (e.g., is separated from at least some cellular material, separated from other polypeptides, separated from its natural sequence context), and/or (ii) is produced by a process that involves the hand of man such as recombinant DNA technology, protein engineering, chemical synthesis, etc.; and/or (iii) has a sequence, structure, or chemical composition not found in nature. “Isolated” is meant to include compounds that are within samples that are substantially enriched for the compound of interest and/or in which the compound of interest is partially or substantially purified. “Purified” as used herein denote that the material referred to is removed from its natural environment and is at least 60% free, at least 75% free, or at least 90% free from other components with which it is naturally associated, also referred to as being “substantially pure”.

The term “sequence identity” as used herein refers to the extent that sequences are identical on a nucleotide-by-nucleotide basis or an amino acid-by-amino acid basis over a window of comparison.

Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Determining the percentage of sequence identity can be done manually, or by making use of computer programs that are available in the art. Examples of useful algorithms are PILEUP (Higgins & Sharp, CABIOS 5:151 (1989), BLAST and BLAST 2.0 (Altschul et al. J. Mol. Biol. 215: 403 (1990). Software for performing BLAST analyses is publicly available through the National Center for Biotechnology Information (http://www.ncbi.nlm.nih.gov/).

“Similarity” refers to the percentage number of amino acids that are identical or constitute conservative substitutions. Similarity may be determined using sequence comparison programs such as GAP (Deveraux et al. 1984). In this way, sequences of a similar or substantially different length to those cited herein might be compared by insertion of gaps into the alignment, such gaps being determined, for example, by the comparison algorithm used by GAP. As used herein, “conservative substitution” is the substitution of amino acids with other amino acids whose side chains have similar biochemical properties (e.g. are aliphatic, are aromatic, are positively charged, . . . ) and is well known to the skilled person. Non-conservative substitution is then the substitution of amino acids with other amino acids whose side chains do not have similar biochemical properties (e.g. replacement of a hydrophobic with a polar residue). Conservative substitutions will typically yield sequences which are not identical anymore, but still highly similar. By conservative substitutions is intended combinations such as gly, ala; val, ile, leu, met; asp, glu; asn, gin; ser, thr; lys, arg; cys, met; and phe, tyr, trp.

A “deletion” is defined here as a change in either amino acid or nucleotide sequence in which one or more amino acid or nucleotide residues, respectively, are absent as compared to an amino acid sequence or nucleotide sequence of a parental polypeptide or nucleic acid. Within the context of a protein, a deletion can involve deletion of about 2, about 5, about 10, up to about 20, up to about 30 or up to about 50 or more amino acids. A protein or a fragment thereof may contain more than one deletion. Within the context of a GPCR, a deletion may also be a loop deletion, or an N- and/or C-terminal deletion. As will be clear to the skilled person, an N- and/or C-terminal deletion of a GPCR is also referred to as a truncation of the amino acid sequence of the GPCR or a truncated GPCR.

An “insertion” or “addition” is that change in an amino acid or nucleotide sequences which has resulted in the addition of one or more amino acid or nucleotide residues, respectively, as compared to an amino acid sequence or nucleotide sequence of a parental protein. “Insertion” generally refers to addition to one or more amino acid residues within an amino acid sequence of a polypeptide, while “addition” can be an insertion or refer to amino acid residues added at an N- or C-terminus, or both termini. Within the context of a protein or a fragment thereof, an insertion or addition is usually of about 1, about 3, about 5, about 10, up to about 20, up to about 30 or up to about 50 or more amino acids. A protein or fragment thereof may contain more than one insertion.

A “substitution”, as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity. By conservative substitutions is intended combinations such as gly, ala; val, ile, leu, met; asp, glu; asn, gin; ser, thr; lys, arg; cys, met; and phe, tyr, trp.

The term “amino acid differences” refers to the total number of amino acid residues in a sequence that have been changed (i.e. by substitution, insertion and/or deletion) compared to a starting or reference sequence. The number of amino acid differences between a sequence and a reference sequence can usually be determined by comparing these sequences, e.g. by making an alignment.

The term “ortholog” when used in reference to an amino acid or nucleotide/nucleic acid sequence from a given species refers to the same amino acid or nucleotide/nucleic acid sequence from a different species. It should be understood that two sequences are orthologs of each other when they are derived from a common ancestor sequence via linear descent and/or are otherwise closely related in terms of both their sequence and their biological function. Orthologs will usually have a high degree of sequence identity but may not (and often will not) share 100% sequence identity.

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express nucleic acids or polypeptides that are not found within the native (non-recombinant) form of the cell or express native genes that are otherwise abnormally expressed, under expressed, over expressed or not expressed at all.

As used herein, the term “expression” refers to the process by which a polypeptide is produced based on the nucleic acid sequence of a gene. The process includes both transcription and translation.

The term “operably linked” as used herein refers to a linkage in which the regulatory sequence is contiguous with the gene of interest to control the gene of interest, as well as regulatory sequences that act in trans or at a distance to control the gene of interest. For example, a DNA sequence is operably linked to a promoter when it is ligated to the promoter downstream with respect to the transcription initiation site of the promoter and allows transcription elongation to proceed through the DNA sequence. A DNA for a signal sequence is operably linked to DNA coding for a polypeptide if it is expressed as a pre-protein that participates in the transport of the polypeptide. Linkage of DNA sequences to regulatory sequences is typically accomplished by ligation at suitable restriction sites or adapters or linkers inserted in lieu thereof using restriction endonucleases known to one of skill in the art.

The term “regulatory sequence” as used herein, and also referred to as “control sequence”, refers to polynucleotide sequences which are necessary to affect the expression of coding sequences to which they are operably linked. Regulatory sequences are sequences which control the transcription, post-transcriptional events and translation of nucleic acid sequences. Regulatory sequences include appropriate transcription initiation, termination, promoter and enhancer sequences; efficient RNA processing signals such as splicing and polyadenylation signals; sequences that stabilize cytoplasmic mRMA; sequences that enhance translation efficiency (e.g., ribosome binding sites); sequences that enhance protein stability; and when desired, sequences that enhance protein secretion. The nature of such control sequences differs depending upon the host organism. The term “regulatory sequence” is intended to include, at a minimum, all components whose presence is essential for expression, and can also include additional components whose presence is advantageous, for example, leader sequences and fusion partner sequences.

The term “vector” as used herein is intended to refer to a nucleic acid molecule capable of transporting another nucleic acid molecule to which it has been linked. The vector may be of any suitable type including, but not limited to, a phage, virus, plasmid, phagemid, cosmid, bacmid or even an artificial chromosome. Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of certain genes of interest. Such vectors are referred to herein as “recombinant expression vectors” (or simply, “expression vectors”). Suitable vectors have regulatory sequences, such as promoters, enhancers, terminator sequences, and the like as desired and according to a particular host organism (e.g. bacterial cell, yeast cell). Typically, a recombinant vector according to the present invention comprises at least one “chimeric gene” or “expression cassette”. Expression cassettes are generally DNA constructs preferably including (5′ to 3′ in the direction of transcription): a promoter region, a polynucleotide sequence, homologue, variant or fragment thereof of the present invention operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell.

The term “host cell”, as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may be an isolated cell or cell line grown in culture or may be a cell which resides in a living tissue or organism. In particular, host cells are of bacterial or fungal origin, but may also be of plant or mammalian origin. The wordings “host cell”, “recombinant host cell”, “expression host cell”, “expression host system”, “expression system”, are intended to have the same meaning and are used interchangeably herein.

“G-protein coupled receptors” or “GPCRs” are polypeptides that share a common structural motif, having an extracellular amino-terminus (N-terminus), an intracellular carboxy terminus (C-terminus) and seven hydrophobic transmembrane seven regions of between 22 to 24 hydrophobic amino acids that form seven alpha helices, each of which spans a membrane. Each span is identified by number, i.e., transmembrane-1 (TM1), transmembrane-2 (TM2), etc. The transmembrane helices are joined by regions of amino acids between transmembrane-2 and transmembrane-3, transmembrane-4 and transmembrane-5, and transmembrane-6 and transmembrane-7 on the exterior, or “extracellular” side, of the cell membrane, referred to as “extracellular” regions 1, 2 and 3 (EC1, EC2 and EC3), respectively. The transmembrane helices are also joined by regions of amino acids between transmembrane-1 and transmembrane-2, transmembrane-3 and transmembrane-4, and transmembrane-5 and transmembrane-6 on the interior, or “intracellular” side, of the cell membrane, referred to as “intracellular” regions 1, 2 and 3 (IC1, IC2 and IC3), respectively. The “carboxy” (“C”) terminus of the receptor lies in the intracellular space within the cell, and the “amino” (“N”) terminus of the receptor lies in the extracellular space outside of the cell. GPCR structure and classification is generally well known in the art, and further discussion of GPCRs may be found in Cvicek et al., PLoS Comput Biol. 2016 Mar. 30; 12(3):e1004805. doi: 10.1371/journal.pcbi.1004805; Ventakakrishnan, Current Opinion in Structural Biology, 2014, 27:129-137; Isberg, Trends Pharmacol. Sci., 2015 January, 22-13, Probst, DNA Cell Biol. 1992 11:1-20; Marchese et al Genomics 23: 609-618, 1994; and the following books: Jurgen Wess (Ed) Structure-Function Analysis of G Protein-Coupled Receptors published by Wiley Liss (1st edition; Oct. 15, 1999); Kevin R. Lynch (Ed) Identification and Expression of G Protein-Coupled Receptors published by John Wiley & Sons (March 1998) and Tatsuya Haga (Ed), G Protein-Coupled Receptors, published by CRC Press (Sep. 24, 1999); and Steve Watson (Ed) G-Protein Linked Receptor Factsbook, published by Academic Press (1st edition; 1994).

The International Union of Basic and Clinical Pharmacology (IUPHAR) maintains a database (http://www.guidetopharmacology.org/targets.jsp) of receptors (including GPCRs) and their known endogenous ligands and signaling mechanisms. According to this database, as of January 2019, about 800 GPCRs have been identified in man, of which about half have sensory functions (for example olfaction, taste, light perception and pheromone signaling) and about half mediate signaling associated with ligands that range in size from small molecules to peptides to large proteins. The IUPHAR database as of January 2019 describes two systems for classifying GPCRs, one of which is based on six classes of GPCRs, as follows: Class A (rhodopsin-like), Class B (secretin receptor family), Class C (metabotropic glutamate), Class D (fungal mating pheromone receptors, not found in vertebrates), Class E (cyclic AMP receptors, also not found in vertebrates) and Class F (frizzled/smoothened). The IUPHAR database also mentions an alternative classification scheme known as “GRAFS” which divides the vertebrate GPCRs into five classes (overlapping with the A-F nomenclature), as follows: the Glutamate family (overlapping with the above “class C”), which inter alia includes metabotropic glutamate receptors, a calcium-sensing receptor and GABAB receptors; the Rhodopsin family (overlapping with the above “class A”), which includes receptors for a wide variety of small molecules, neurotransmitters, peptides and hormones, together with olfactory receptors, visual pigments, taste type 2 receptors and five pheromone receptors (V1 receptors); the Adhesion family GPCRs (which are phylogenetically related to class B receptors); the Frizzled family, consisting of 10 Frizzled proteins (FZD(1-10)) and Smoothened (SMO); and the Secretin family, which are receptors for peptide ligands/hormones having between 27-141 amino acid residues, including glucagon, glucagon-like peptides (GLP-1, GLP-2), glucose-dependent insulinotropic polypeptide (GIP), secretin, vasoactive intestinal peptide (VIP), pituitary adenylate cyclase-activating polypeptide (PACAP) and growth-hormone-releasing hormone (GHRH). In this description and in the appended claims, the Type A-to-F classification will be used, unless explicitly stated otherwise. Further reference is made to Cvicek et al., cited herein.

The term “biologically active”, with respect to a GPCR, refers to a GPCR having a biochemical function (e.g., a binding function, a signal transduction function, or an ability to change conformation as a result of ligand binding) of a naturally occurring GPCR.

In general, the term “naturally-occurring” in reference to a GPCR means a GPCR that is naturally produced (e.g., by a wild type mammal such as a human). Such GPCRs are found in nature. The term “non-naturally occurring” in reference to a GPCR means a GPCR that is not naturally-occurring. Naturally-occurring GPCRs that have been made constitutively active through mutation, and variants of naturally-occurring transmembrane receptors, e.g., epitope-tagged GPCRs and GPCRs lacking their native N-terminus are examples of non-naturally occurring GPCRs. Non-naturally occurring versions of a naturally occurring GPCR are often activated by the same ligand as the naturally-occurring GPCR. Non-limiting examples of either naturally-occurring or non-naturally occurring GPCRs within the context of the present invention are provided further herein.

An “epitope”, as used herein, refers to an antigenic determinant of a polypeptide. An epitope could comprise 3 amino acids in a spatial conformation, which is unique to the epitope. Generally an epitope consists of at least 4, 5, 6, 7 such amino acids, and more usually, consists of at least 8, 9, 10 such amino acids. Methods of determining the spatial conformation of amino acids are known in the art, and include, for example, x-ray crystallography and multi-dimensional nuclear magnetic resonance. A “conformational epitope”, as used herein, refers to an epitope comprising amino acids in a spacial conformation that is unique to a folded 3-dimensional conformation of the polypeptide. Generally, a conformational epitope consists of amino acids that are discontinuous in the linear sequence that come together in the folded structure of the protein. However, a conformational epitope may also consist of a linear sequence of amino acids that adopts a conformation that is unique to a folded 3-dimensional conformation of the polypeptide (and not present in a denatured state).

The term “conformation” or “conformational state” of a protein refers generally to a spacial arrangement, structure or range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Post-translational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

A “functional conformation” or a “functional conformational state”, as used herein, refers to the fact that proteins possess different conformational states having a dynamic range of activity, in particular ranging from no activity to maximal activity. It should be clear that “a functional conformational state” is meant to cover any conformational state of a protein, having any activity, including no activity, and is not meant to cover the denatured states of proteins. Non-limiting examples of functional conformations include active conformations, inactive conformations or basal conformations (as defined further herein). As mentioned, a particular class of functional conformations is defined as “druggable conformation” and generally refers to the therapeutically relevant conformational state(s) of the protein. Reference is for example made to Johnson and Karanicolas, PLoS Comput Biol 9(3): e1002951. doi:10.1371/journal.pcbi.1002951 and to for example WO2014/122183 which describes that the agonist-bound active conformation of the muscarinic acetylcholine receptor M2 corresponds to the druggable conformation of this receptor relating to pain and gliobastoma, and describes VHHs that can stabilize said druggable conformation for assay and screening purposes. It will thus be understood that druggability is confined to particular conformations depending on the therapeutic indication. More details are provided further herein.

With respect to a protein that is a receptor, the term “active conformation”, as used herein, more specifically refers to a conformation or spectrum of receptor conformations that allows signal transduction towards an intracellular effector system, such as G protein dependent signaling and/or G protein-independent signaling (e.g. β-arrestin signaling). An “active conformation” thus encompasses a range of ligand-specific conformations, including an agonist-specific active state conformation, a partial agonist-specific active state conformation or a biased agonist-specific active state conformation, so that it induces the cooperative binding of an intracellular effector protein.

In addition to the foregoing, with respect to a GPCR, the terms “active conformation” and “active form” as used herein refer to a GPCR that is folded in a way so as to be (functionally) active. A GPCR can be placed into an active conformation using an activating ligand (agonist) of the receptor, and such a conformational change will generally enable the receptor to activate heterotrimeric G proteins. For example, a GPCR in its active conformation binds to heterotrimeric G protein and catalyzes nucleotide exchange of the G-protein to activate downstream signaling pathways. Activated GPCRs bind to the inactive, GDP-bound form of heterotrimeric G-proteins and cause the G-proteins to release their GDP so GTP can bind. There is a transient ‘nucleotide-free’ state that results from this process that enables GTP to bind. Once GTP is bound, the receptor and G-protein dissociate, allowing the GTP-bound G protein to activate downstream signaling pathways such as adenylyl cyclase, ion channels, RAS/MAPK, etc. The terms “inactive conformation” and “inactive form” refer to a GPCR that is folded in a way so as to be inactive. A GPCR can be placed into an inactive conformation using an inverse agonist of the receptor. For example, a GPCR in its inactive conformation does not bind to heterotrimeric G protein with high affinity. The terms “active conformation” and “inactive conformation” will be illustrated further herein. As used herein, the term “basal conformation” refers to a GPCR that is folded in a way that it exhibits activity towards a specific signaling pathway even in the absence of an agonist (also referred to as basal activity or constitutive activity). Inverse agonists can inhibit this basal activity. Thus, a basal conformation of a GPCR corresponds to a stable conformation or prominent structural species in the absence of ligands or accessory proteins.

Similarly, with respect to a protein that is a receptor, the term “inactive conformation” as used herein refers to a spectrum of receptor conformations that does not allow or blocks signal transduction towards an intracellular effector system. An “inactive conformation” thus encompasses a range of ligand-specific conformations, including an inverse agonist-specific inactive state conformation, so that it prevents the cooperative binding of an intracellular effector protein. It will be understood that the site of binding of the ligand is not critical for obtaining an active or inactive conformation. Hence, orthosteric ligands as well as allosteric modulators may equally be capable of stabilizing a receptor in an active or inactive conformation.

The term “binding agent”, as used herein, means the whole or part of a proteinaceous (protein, protein-like or protein containing) molecule that is capable of binding using specific intermolecular interactions to a membrane protein. In a particular embodiment, the term “binding agent” is not meant to include a naturally-occurring binding partner of the relevant membrane protein, such as a G protein, an arrestin, an endogenous ligand; or variants or derivatives (including fragments) thereof. More specifically, the term “binding agent” refers to a polypeptide, more particularly a protein domain. A suitable protein domain is an element of overall protein structure that is self-stabilizing and that folds independently of the rest of the protein chain and is often referred to as “binding domain”. Such binding domains vary in length from between about 25 amino acids up to 500 amino acids and more. Many binding domains can be classified into folds and are recognizable, identifiable, 3-D structures. Some folds are so common in many different proteins that they are given special names. Non-limiting examples are binding domains selected from a 3- or 4-helix bundle, an armadillo repeat domain, a leucine-rich repeat domain, a PDZ domain, a SUMO or SUMO-like domain, a cadherin domain, an immunoglobulin-like domain, phosphotyrosine-binding domain, pleckstrin homology domain, src homology 2 domain, amongst others. A binding domain can thus be derived from a naturally occurring molecule, e.g. from components of the innate or adaptive immune system, or it can be entirely artificially designed.

In general, a binding domain can be immunoglobulin-based or it can be based on domains present in proteins, including but limited to microbial proteins, protease inhibitors, toxins, fibronectin, lipocalins, single chain antiparallel coiled coil proteins or repeat motif proteins. Particular examples of binding domains which are known in the art include, but are not limited to: antibodies, heavy chain antibodies (hcAb), single domain antibodies (sdAb), minibodies, the variable domain derived from camelid heavy chain antibodies (VHH or Nanobodies), the variable domain of the new antigen receptors derived from shark antibodies (VNA), alphabodies, protein A, protein G, designed ankyrin-repeat domains (DARPins), fibronectin type III repeats, anticalins, knottins, engineered CH2 domains (nanoantibodies), engineered SH3 domains, affibodies, peptides and proteins, lipopeptides (e.g. pepducins) (see, e.g., Gebauer & Skerra, 2009; Skerra, 2000; Starovasnik et al., 1997; Binz et al., 2004; Koide et al., 1998; Dimitrov, 2009; Nygren et al. 2008; WO2010066740). Frequently, when generating a particular type of binding domain using selection methods, combinatorial libraries comprising a consensus or framework sequence containing randomized potential interaction residues are used to screen for binding to a molecule of interest, such as a protein.

According to a preferred embodiment, it is particularly envisaged that the binding agent of the invention is derived from an innate or adaptive immune system. Preferably, said binding agent is derived from an immunoglobulin. Preferably, the binding agent according to the invention is derived from an antibody or an antibody fragment. The term “antibody” (Ab) refers generally to a polypeptide encoded by an immunoglobulin gene, or a functional fragment thereof, that specifically binds and recognizes an antigen, and is known to the person skilled in the art. An antibody is meant to include a conventional four-chain immunoglobulin, comprising two identical pairs of polypeptide chains, each pair having one “light” (about 25 kDa) and one “heavy” chain (about 50 kDa). Typically, in conventional immunoglobulins, a heavy chain variable domain (VH) and a light chain variable domain (VL) interact to form an antigen binding site. The term “antibody” is meant to include whole antibodies, including single-chain whole antibodies, and antigen-binding fragments. In some embodiments, antigen-binding fragments may be antigen-binding antibody fragments that include, but are not limited to, Fab, Fab′ and F(ab′)2, Fd, single-chain Fvs (scFv), single-chain antibodies, disulfide-linked Fvs (dsFv) and fragments comprising or consisting of either a VL or VH domain, and any combination of those or any other functional portion of an immunoglobulin peptide capable of binding to the target antigen. The term “antibodies” is also meant to include heavy chain antibodies, or fragments thereof, including immunoglobulin single variable domains, as defined further herein.

The term “immunoglobulin single variable domain” or “ISVD” defines molecules wherein the antigen binding site is present on, and formed by, a single immunoglobulin domain (which is different from conventional immunoglobulins or their fragments, wherein typically two immunoglobulin variable domains interact to form an antigen binding site). It should however be clear that the term “immunoglobulin single variable domain” does comprise fragments of conventional immunoglobulins wherein the antigen binding site is formed by a single variable domain. Preferably, the binding agent within the scope of the present invention is an immunoglobulin single variable domain.

Generally, an immunoglobulin single variable domain will be an amino acid sequence comprising 4 framework regions (FR1 to FR4) and 3 complementary determining regions (CDR1 to CDR3), preferably according to the following formula (1): FR1-CDR1-FR2-CDR2-FR3-CDR3-FR4 (1), or any suitable fragment thereof (which will then usually contain at least some of the amino acid residues that form at least one of the complementarity determining regions). ISVDs comprising 4 FRs and 3 CDRs are known to the person skilled in the art and have been described, as a non-limiting example, in Wesolowski et al. 2009. Typical, but non-limiting, examples of immunoglobulin single variable domains include light chain variable domain sequences (e.g. a VL domain sequence) or a suitable fragment thereof, or heavy chain variable domain sequences (e.g. a VH domain sequence or VHH domain sequence) or a suitable fragment thereof, as long as it is capable of forming a single antigen binding unit. Thus, according to a preferred embodiment, the binding agent is an immunoglobulin single variable domain that is a light chain variable domain sequence (e.g. a VL domain sequence) or a heavy chain variable domain sequence (e.g. a VH domain sequence); more specifically, the immunoglobulin single variable domain is a heavy chain variable domain sequence that is derived from a conventional four-chain antibody or a heavy chain variable domain sequence that is derived from a heavy chain antibody. The immunoglobulin single variable domain may be a domain antibody, or a single domain antibody, or a “dAB” or dAb, or a Nanobody (as defined herein), or another immunoglobulin single variable domain, or any suitable fragment of any one thereof. For a general description of single domain antibodies, reference is made to the following book: “Single domain antibodies”, Methods in Molecular Biology, Eds. Saerens and Muyldermans, 2012, Vol 911. The immunoglobulin single variable domains, generally comprise a single amino acid chain that can be considered to comprise 4 “framework sequences” or FR's and 3 “complementary determining regions” or CDR's (as defined hereinbefore). It should be clear that framework regions of immunoglobulin single variable domains may also contribute to the binding of their antigens (Desmyter et al 2002; Korotkov et al. 2009).

As further described herein, the total number of amino acid residues in a VHH, Nanobody or ConfoBody can be in the region of 110-120, is preferably 112-115, and is most preferably 113. It should however be noted that parts, fragments, analogs or derivatives (as further described herein) of a VHH or Nanobody are not particularly limited as to their length and/or size, as long as such parts, fragments, analogs or derivatives meet the further requirements outlined herein and are also preferably suitable for the purposes described herein.

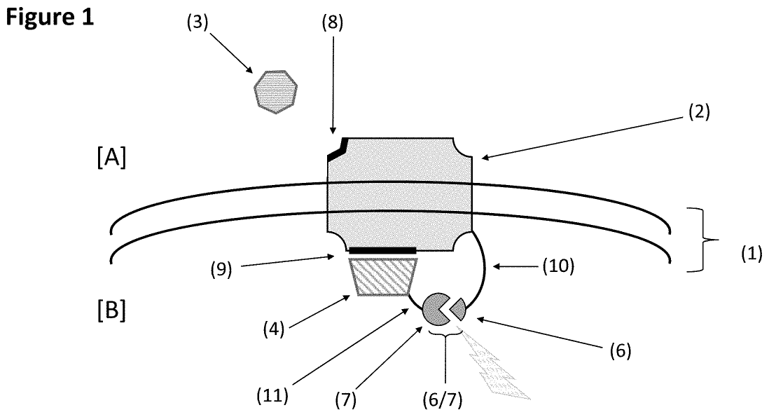

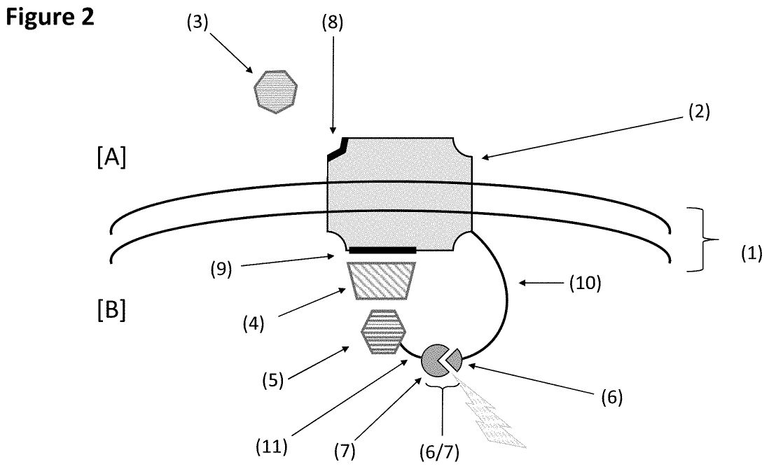

In the present application, the amino acid residues/positions in an immunoglobulin heavy-chain variable domain will be indicated with the numbering according to Kabat (“Sequence of proteins of immunological interest”, US Public Health Services, NIH Bethesda, Md., Publication No. 91), as applied to V_{HH} domains from Camelids in the article of Riechmann and Muyldermans, J. Immunol. Methods 2000 Jun. 23; 240 (1-2): 185-195 (see for example FIG. 2 of this publication). Reference is for example also made to FIG. 1 of the International application WO 2108/134235, which gives a table listing some of the amino acid positions in a VHH and their numbering according to some alternative numbering systems (such as Aho and IMGT. Note: unless explicitly indicated otherwise, for the present description and claims, Kabat numbering is decisive for the amino acid residues/positions in VHH, Nanobody or ConfoBody; other numbering systems are given for reference only).

With regard to the CDR's, as is well-known in the art, there are multiple conventions to define and describe the CDR's of a VH or VHH fragment, such as the Kabat definition (which is based on sequence variability and is the most commonly used) and the Chothia definition (which is based on the location of the structural loop regions). Reference is for example made to the website http://www.bioinf.org.uk/abs/). For the purposes of the present specification and claims, even though the CDR's according to Kabat may also be mentioned, the CDRs are most preferably defined on the basis of the Abm definition (which is based on Oxford Molecular's AbM antibody modelling software), as this is considered to be an optimal compromise between the Kabat and Chothia definitions. Reference is again made to the website http://www.bioinf.org.uk/abs/).

Accordingly, in the present specification and claims, all CDRs or a VHH, Nanobody or ConfoBody are defined according to the Abm convention, unless explicitly stated otherwise herein.

It should be noted that the immunoglobulin single variable domains as binding agent in their broadest sense are not limited to a specific biological source or to a specific method of preparation. The term “immunoglobulin single variable domain” or “ISVD” encompasses variable domains of different origin, comprising mouse, rat, rabbit, donkey, human, shark, camelid variable domains. According to specific embodiments, the immunoglobulin single variable domains are derived from shark antibodies (the so-called immunoglobulin new antigen receptors or IgNARs), more specifically from naturally occurring heavy chain shark antibodies, devoid of light chains, and are known as VNAR domain sequences. Preferably, the immunoglobulin single variable domains are derived from camelid antibodies. More preferably, the immunoglobulin single variable domains are derived from naturally occurring heavy chain camelid antibodies, devoid of light chains, and are known as VHH domain sequences or Nanobodies.

According to a particularly preferred embodiment, the binding agent of the invention is an immunoglobulin single variable domain that is a Nanobody (as defined further herein, and including but not limited to a VHH). The term “Nanobody” (Nb), as used herein, is a single domain antigen binding fragment. It particularly refers to a single variable domain derived from naturally occurring heavy chain antibodies and is known to the person skilled in the art. Nanobodies are usually derived from heavy chain only antibodies (devoid of light chains) seen in camelids (Hamers-Casterman et al. 1993; Desmyter et al. 1996) and consequently are often referred to as VHH antibody or VHH sequence. Camelids comprise old world camelids (Camelus bactrianus and Camelus dromedarius) and new world camelids (for example Lama paccos, Lama glama, Lama guanicoe and Lama vicugna). Nanobody® and Nanobodies® are registered trademarks of Ablynx NV (Belgium). For a further description of VHH's or Nanobodies, reference is made to the book “Single domain antibodies”, Methods in Molecular Biology, Eds. Saerens and Muyldermans, 2012, Vol 911, in particular to the Chapter by Vincke and Muyldermans (2012), as well as to a non-limiting list of patent applications, which are mentioned as general background art, and include: WO 94/04678, WO 95/04079, WO 96/34103 of the Vrije Universiteit Brussel; WO 94/25591, WO 99/37681, WO 00/40968, WO 00/43507, WO 00/65057, WO 01/40310, WO 01/44301, EP 1 134 231 and WO 02/48193 of Unilever; WO 97/49805, WO 01/21817, WO 03/035694, WO 03/054016 and WO 03/055527 of the Vlaams Instituut voor Biotechnologie (VIB); WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As will be known by the person skilled in the art, the Nanobodies are particularly characterized by the presence of one or more Camelidae “hallmark residues” in one or more of the framework sequences (according to Kabat numbering), as described for example in WO 08/020079, on page 75, Table A-3, incorporated herein by reference). It should be noted that the Nanobodies, of the invention in their broadest sense are not limited to a specific biological source or to a specific method of preparation. For example, Nanobodies, can generally be obtained: (i) by isolating the VHH domain of a naturally occurring heavy chain antibody; (ii) by expression of a nucleotide sequence encoding a naturally occurring VHH domain; (iii) by “humanization” of a naturally occurring VHH domain or by expression of a nucleic acid encoding a such humanized VHH domain; (iv) by “camelization” of a naturally occurring VH domain from any animal species, and in particular from a mammalian species, such as from a human being, or by expression of a nucleic acid encoding such a camelized VH domain; (v) by “camelisation” of a “domain antibody” or “Dab” as described in the art, or by expression of a nucleic acid encoding such a camelized VH domain; (vi) by using synthetic or semi-synthetic techniques for preparing proteins, polypeptides or other amino acid sequences known per se; (vii) by preparing a nucleic acid encoding a Nanobody using techniques for nucleic acid synthesis known per se, followed by expression of the nucleic acid thus obtained; and/or (8) by any combination of one or more of the foregoing. A further description of Nanobodies, including humanization and/or camelization of Nanobodies, can be found e.g. in WO08/101985 and WO08/142164, as well as further herein. A particular class of Nanobodies binding conformational epitopes of native targets is called Xaperones and is particularly envisaged here. Xaperone™ is a trademark of VIB and VUB (Belgium). A Xaperone™ is a camelid single domain antibody that constrains drug targets into a unique, disease relevant druggable conformation.

Within the scope of the present invention, the term “immunoglobulin single variable domain” also encompasses variable domains that are “humanized” or “camelized”, in particular Nanobodies that are “humanized” or “camelized”. For example both “humanization” and “camelization” can be performed by providing a nucleotide sequence that encodes a naturally occurring VHH domain or VH domain, respectively, and then changing, in a manner known per se, one or more codons in said nucleotide sequence in such a way that the new nucleotide sequence encodes a “humanized” or “camelized” immunoglobulin single variable domains of the invention, respectively. This nucleic acid can then be expressed in a manner known per se, so as to provide the desired immunoglobulin single variable domains of the invention. Alternatively, based on the amino acid sequence of a naturally occurring VHH domain or VH domain, respectively, the amino acid sequence of the desired humanized or camelized immunoglobulin single variable domains of the invention, respectively, can be designed and then synthesized de novo using techniques for peptide synthesis known per se. Also, based on the amino acid sequence or nucleotide sequence of a naturally occurring VHH domain or VH domain, respectively, a nucleotide sequence encoding the desired humanized or camelized immunoglobulin single variable domains of the invention, respectively, can be designed and then synthesized de novo using techniques for nucleic acid synthesis known per se, after which the nucleic acid thus obtained can be expressed in a manner known per se, so as to provide the desired immunoglobulin single variable domains of the invention. Other suitable methods and techniques for obtaining the immunoglobulin single variable domains of the invention and/or nucleic acids encoding the same, starting from naturally occurring VH sequences or preferably VHH sequences, will be clear from the skilled person, and may for example comprise combining one or more parts of one or more naturally occurring VH sequences (such as one or more FR sequences and/or CDR sequences), one or more parts of one or more naturally occurring VHH sequences (such as one or more FR sequences or CDR sequences), and/or one or more synthetic or semi-synthetic sequences, in a suitable manner, so as to provide a Nanobody of the invention or a nucleotide sequence or nucleic acid encoding the same.

According to a particular embodiment of the present invention, the binding agent that is capable of stabilizing the receptor may bind at the orthosteric site or an allosteric site. In other specific embodiments, the binding agent that is capable of stabilizing the receptor may be an active conformation-selective binding agent, or an inactive conformation-selective binding agent, either by binding at the orthosteric site or at an allosteric site. Generally, a conformation-selective binding agent that stabilizes an active conformation of a receptor will increase or enhance the affinity of the receptor for an active conformation-selective ligand, such as an agonist, more specifically a full agonist, a partial agonist or a biased agonist, as compared to the receptor in the absence of the binding agent (or in the presence of a mock binding agent—also referred to as control binding agent or irrelevant binding agent—that is not directed against and/or does not specifically bind to the receptor). Also, a binding agent that stabilizes an active conformation of a receptor will decrease the affinity of the receptor for an inactive conformation-selective ligand, such as an inverse agonist, as compared to the receptor in the absence of the binding agent (or in the presence of a mock binding agent). In contrast, a binding agent that stabilizes an inactive conformation of a receptor will enhance the affinity of the receptor for an inverse agonist and will decrease the affinity of the receptor for an agonist, particularly for a full agonist, a partial agonist or a biased agonist, as compared to the receptor in the absence of the binding agent (or in the presence of a mock binding agent). An increase or decrease in affinity for a ligand may be directly measured by and/or calculated from a decrease or increase, respectively in EC50, IC50, Kd, K, or any other measure of affinity or potency known to one of skill in the art. It is particularly preferred that the binding agent that stabilizes a particular conformation of a receptor is capable of increasing or decreasing the affinity for a conformation-selective ligand at least 2 fold, at least 5 fold, at least 10 fold, at least 50 fold, and more preferably at least 100 fold, even more preferably at least 1000 fold or more, upon binding to the receptor. It will be appreciated that affinity measurements for conformation-selective ligands that trigger/inhibit particular signaling pathways may be carried out with any type of ligand, including natural ligands, small molecules, as well as biologicals; with orthosteric ligands as well as allosteric modulators; with single compounds as well as compound libraries; with lead compounds or fragments; etc.

The term “affinity”, as used herein, refers to the degree to which a ligand (as defined further herein) binds to a target protein so as to shift the equilibrium of target protein and ligand toward the presence of a complex formed by their binding. Thus, for example, where a GPCR and a ligand are combined in relatively equal concentration, a ligand of high affinity will bind to the available antigen on the GPCR so as to shift the equilibrium toward high concentration of the resulting complex. The dissociation constant is commonly used to describe the affinity between a ligand and a target protein. Typically, the dissociation constant is lower than 10⁻⁵ M. Preferably, the dissociation constant is lower than 10⁻⁶ M, more preferably, lower than 10⁻⁷ M. Most preferably, the dissociation constant is lower than 10⁻⁸M. Other ways of describing the affinity between a ligand (including small molecule ligands) and its target protein are the association constant (Ka), the inhibition constant (Ki) (also referred to as the inhibitory constant), or indirectly by evaluating the potency of ligands by measuring the half maximal inhibitory concentration (IC50) or half maximal effective concentration (EC50). Within the scope of the present invention, the ligand may be a binding agent, preferably an immunoglobulin, such as an antibody, or an immunoglobulin fragment, such as a VHH or Nanobody, that binds a conformational epitope on a GPCR. It will be appreciated that within the scope of the present invention, the term “affinity” is used in the context of a binding agent, in particular an immunoglobulin or an immunoglobulin fragment, such as a VHH or Nanobody, that binds a conformational epitope of a target GPCR as well as in the context of a test compound (as defined further herein) that binds to a target GPCR, more particularly to an orthosteric or allosteric site of a target GPCR.

The term “specificity”, as used herein, refers to the ability of a protein or other binding agent, in particular an immunoglobulin or an immunoglobulin fragment, such as a VHH or Nanobody, to bind preferentially to one antigen, versus a different antigen, and does not necessarily imply high affinity.

The terms “specifically bind” and “specific binding”, as used herein, generally refers to the ability of a binding agent, in particular an immunoglobulin, such as an antibody, or an immunoglobulin fragment, such as a VHH or Nanobody, to preferentially bind to a particular antigen that is present in a homogeneous mixture of different antigens. In certain embodiments, a specific binding interaction will discriminate between desirable and undesirable antigens in a sample, in some embodiments more than about 10 to 100-fold or more (e.g., more than about 1000- or 10,000-fold). Within the context of the spectrum of conformational states of GPCRs, the terms particularly refer to the ability of a binding agent (as defined herein) to preferentially recognize and/or bind to a particular conformational state of a GPCR as compared to another conformational state.

Also, it should be understood that in the present description and appended claims, where a protein, ligand, compound, binding domain, binding unit or other chemical entity is said to “bind” another protein, ligand, compound, binding domain, binding unit or other chemical entity or an epitope or binding site, that such binding is preferably “specific” binding as defined herein. Also, preferably, such binding is “selective binding” as defined herein.

As used herein, the term “conformation-selective binding agent” in the context of the present invention refers to a binding agent that binds to a target protein in a conformation-selective manner. A binding agent that selectively binds to a particular conformation or conformational state of a protein refers to a binding agent that binds with a higher affinity to a protein in a subset of conformations or conformational states than to other conformations or conformational states that the protein may assume. One of skill in the art will recognize that binding agents that selectively bind to a specific conformation or conformational state of a protein will stabilize or retain the protein it this particular conformation or conformational state. For example, an active conformation-selective binding agent will preferentially bind to a GPCR in an active conformational state and will not or to a lesser degree bind to a GPCR in an inactive conformational state, and will thus have a higher affinity for said active conformational state; or vice versa. The terms “specifically bind”, “selectively bind”, “preferentially bind”, and grammatical equivalents thereof, are used interchangeably herein. The terms “conformational specific” or “conformational selective” are also used interchangeably herein.

As used herein, the term “stabilizing”, or grammatically equivalent terms, as defined hereinbefore, is meant an increased stability of a protein (as described herein) or receptor (also as described herein) with respect to the structure (e.g. conformational state) and/or particular biological activity (e.g. intracellular signaling activity, ligand binding affinity, . . . ). In relation to increased stability with respect to structure and/or biological activity, this may be readily determined by either a functional assay for activity (e.g. Ca2+ release, cAMP generation or transcriptional activity, β-arrestin recruitment, . . . ) or ligand binding or by means of physical methods such as X-ray crystallography, NMR, or spin labeling, among other methods. The term “stabilize” also includes increased thermostability of the receptor under non-physiological conditions induced by denaturants or denaturing conditions. The term “thermostabilize”, “thermostabilizing”, “increasing the thermostability of”, as used herein, refers to the functional rather than to the thermodynamic properties of a receptor and to the protein's resistance to irreversible denaturation induced by thermal and/or chemical approaches including but not limited to heating, cooling, freezing, chemical denaturants, pH, detergents, salts, additives, proteases or temperature. Irreversible denaturation leads to the irreversible unfolding of the functional conformations of the protein, loss of biological activity and aggregation of the denaturated protein. In relation to an increased stability to heat, this can be readily determined by measuring ligand binding or by using spectroscopic methods such as fluorescence, CD or light scattering that are sensitive to unfolding at increasing temperatures. It is preferred that the binding agent is capable of increasing the stability as measured by an increase in the thermal stability of a protein or receptor in a functional conformational state with at least 2° C., at least 5° C., at least 8° C., and more preferably at least 10° C. or 15° C. or 20° C. In relation to an increased stability to a detergent or to a chaotrope, typically the protein or receptor is incubated for a defined time in the presence of a test detergent or a test chaotropic agent and the stability is determined using, for example, ligand binding or a spectroscoptic method, optionally at increasing temperatures as discussed above. Otherwise, the binding agent is capable of increasing the stability to extreme pH of a functional conformational state of a protein or receptor. In relation to an extreme of pH, a typical test pH would be chosen for example in the range 6 to 8, the range 5.5 to 8.5, the range 5 to 9, the range 4.5 to 9.5, more specifically in the range 4.5 to 5.5 (low pH) or in the range 8.5 to 9.5 (high pH). The term “(thermo)stabilize”, “(thermo)stabilizing”, “increasing the (thermo)stability of”, as used herein, applies to protein or receptors embedded in lipid particles or lipid layers (for example, lipid monolayers, lipid bilayers, and the like) and to proteins or receptors that have been solubilized in detergent.

In addition to the foregoing, with respect to a functional conformational state of a GPCR, the term “stabilizing” or “stabilized” refers to the retaining or holding of a GPCR protein in a subset of the possible conformations that it could otherwise assume, due to the effects of the interaction of the GPCR with the binding agent according to the invention. Within this context, a binding agent that selectively binds to a specific conformation or conformational state of a protein refers to a binding agent that binds with a higher affinity to a protein in a subset of conformations or conformational states than to other conformations or conformational states that the protein may assume. One of skill in the art will recognize that binding agents that specifically or selectively bind to a specific conformation or conformational state of a protein will stabilize this specific conformation or conformational state, and its related activity. More details are provided further herein.

The term “compound” or “test compound” or “candidate compound” or “drug candidate compound” as used herein describes any molecule, either naturally occurring or synthetic that is tested in an assay, such as a screening assay or drug discovery assay. As such, these compounds comprise organic or inorganic compounds. The compounds include polynucleotides, lipids or hormone analogs that are characterized by low molecular weights. Other biopolymeric organic test compounds include small peptides or peptide-like molecules (peptidomimetics) comprising from about 2 to about 40 amino acids and larger polypeptides comprising from about 40 to about 500 amino acids, such as antibodies, antibody fragments or antibody conjugates. Test compounds can also be protein scaffolds. For high-throughput purposes, test compound libraries may be used, such as combinatorial or randomized libraries that provide a sufficient range of diversity. Examples include, but are not limited to, natural compound libraries, allosteric compound libraries, peptide libraries, antibody fragment libraries, synthetic compound libraries, fragment-based libraries, phage-display libraries, and the like. A more detailed description can be found further in the specification.

As used herein, the term “ligand” means a molecule that specifically binds to a protein referred to herein, such as to a GPCR. A ligand may be, without the purpose of being limitative, a polypeptide, a lipid, a small molecule, an antibody, an antibody fragment, a nucleic acid, a carbohydrate. A ligand may be synthetic or naturally occurring. A ligand also includes a “native ligand” which is a ligand that is an endogenous, natural ligand for a native GPCR. Within the context of the present invention, when a protein is a transmembrane protein such as a GPCR, a ligand may bind to said protein either on a ligand binding site that is exposed to the intracellular environment when the protein is in its native cellular environment (i.e. the ligand may be an “intracellular ligand”), or the ligand may bind to said protein on a ligand binding site that is exposed to the environment outside of the cell when the protein is in its native cellular environment (i.e. the ligand may be an “extracellular ligand”). A ligand may be an agonist, a partial agonist, an inverse agonist, an antagonist, an allosteric modulator, and may bind at either the orthosteric site or at an allosteric site. In particular embodiments, a ligand may be a “conformation-selective ligand” or “conformation-specific ligand”, meaning that such a ligand binds the protein or GPCR in a conformation-selective manner. As further described herein, a conformation-selective ligand binds with a higher affinity to a particular conformation of the protein than to other conformations the protein may adopt. For the purpose of illustration, an agonist is an example of an active conformation-selective ligand, whereas an inverse agonist is an example of an inactive conformation-selective ligand. For the sake of clarity, a neutral antagonist is not considered as a conformation-selective ligand, since a neutral antagonist does not distinguish between the different conformations of a GPCR.

An “orthosteric ligand”, as used herein, refers to a ligand (both natural and synthetic), that binds to the active site of a GPCR, and are further classified according to their efficacy or in other words to the effect they have on signaling through a specific pathway. As used herein, an “agonist” refers to a ligand that, by binding a receptor protein, increases the receptor's signaling activity. Full agonists are capable of maximal protein stimulation; partial agonists are unable to elicit full activity even at saturating concentrations. Partial agonists can also function as “blockers” by preventing the binding of more robust agonists. An “antagonist”, also referred to as a “neutral antagonist”, refers to a ligand that binds a receptor without stimulating any activity. An “antagonist” is also known as a “blocker” because of its ability to prevent binding of other ligands and, therefore, block agonist-induced activity. Further, an “inverse agonist” refers to an antagonist that, in addition to blocking agonist effects, reduces a receptor's basal or constitutive activity below that of the unliganded protein.

Ligands as used herein may also be “biased ligands” with the ability to selectively stimulate a subset of a receptor's signaling activities, for example in the case of GPCRs the selective activation of G-protein or β-arrestin function. Such ligands are known as “biased ligands”, “biased agonists” or “functionally selective agonists”. More particularly, ligand bias can be an imperfect bias characterized by a ligand stimulation of multiple receptor activities with different relative efficacies for different signals (non-absolute selectivity) or can be a perfect bias characterized by a ligand stimulation of one receptor protein activity without any stimulation of another known receptor protein activity.

Another kind of ligands is known as allosteric regulators. “Allosteric regulators” or otherwise “allosteric modulators”, “allosteric ligands” or “effector molecules”, as used herein, refer to ligands that bind at an allosteric site (that is, a regulatory site physically distinct from the protein's active site) of a GPCR. In contrast to orthosteric ligands, allosteric modulators are non-competitive because they bind receptor proteins at a different site and modify their function even if the endogenous ligand also is binding. Allosteric regulators that enhance the protein's activity are referred to herein as “allosteric activators” or “positive allosteric modulators” (PAMs), whereas those that decrease the protein's activity are referred to herein as “allosteric inhibitors” or otherwise “negative allosteric modulators” (NAMs).

As used herein, the terms “determining”, “measuring”, “assessing”, “assaying” are used interchangeably and include both quantitative and qualitative determinations.

The term “antibody” is intended to mean an immunoglobulin or any fragment thereof that is capable of antigen binding. The term “antibody” also refers to single chain antibodies and antibodies with only one binding domain.

As used herein, the terms “complementarity determining region” or “CDR” within the context of antibodies refer to variable regions of either H (heavy) or L (light) chains (also abbreviated as VH and VL, respectively) and contains the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed LI, L2, L3, HI, H2, H3) for the respective light (L) and heavy (H) chains. Immunoglobulin single variable domains, in particular Nanobodies, generally comprise a single amino acid chain that can be considered to comprise 4 “framework sequences or regions” or FRs and 3 “complementary determining regions” or CDRs. The nanobodies have 3 CDR regions, each non-contiguous with the others (termed CDR1, CDR2, CDR3). As mentioned herein, for denoting the amino acid positions/residues CDRs in a VHH, Nanobody or ConfoBody, the Kabat numbering system will be followed, and the frameworks and CDRs are defined on the basis of the Abm definitions (unless explicitly stated otherwise).

Generally, for the purposes of the disclosure herein and its appended claims, a compound of the invention will be considered to a “modulator” of a target, or to “modulate” a target (and/or of the signaling, pathway(s), mechanism of action and/or said biological, physiological and/or pharmacological functions in which said target is involved) when the presence of the compound (i.e. in a suitable amount or concentration, such as a biologically active amount or concentration) in a suitable assay or model changes a suitable or intended read-out of said assay or model (i.e. at least one suitable value or parameter that can be determined using said assay or model) by at least 0.1%, such as at least 1%, for example at least 10% and up to 50% or more, compared to the same value or parameter when it is measured using the same assay or model under essentially the same conditions but without the presence of said compound. Again, said modulation may result in an increase or a decrease of said value or parameter (i.e. by the percentages given in the previous sentence). Also, a compound of the invention will preferably be such that it can modulate said target, signaling, pathway(s), mechanism of action and/or said biological, physiological and/or pharmacological functions in a dose-dependent manner, i.e. in or over at least one range of concentrations of the compound used in the assay or model.

The methods of the invention are generally performed in an arrangement which comprises at least the following elements (all as further defined herein):
a boundary layer that separates a first environment from a second environment;
a translayer protein;
a first ligand for the translayer protein that is present in (as defined herein) the first environment;
a second ligand for the translayer protein that is present in (as defined herein) the second environment; and
a binding pair that consists of at least a first binding member and a second binding member, which binding pair is capable of generating a detectable signal;
   which elements are arranged with respect to each other (and where applicable operably linked to and/or associated with each other) in the manner as further described herein.

In particular, in the arrangements of the invention and as further described herein, the first binding member of the binding pair may be part of a “first fusion protein” (as further described herein) and the second member of the binding pair may be part of a “second fusion protein” (also as further described herein, and different from the first fusion protein), and such a first fusion protein, such a second fusion protein (in its various formats as described herein), nucleotide sequences and/or nucleic acids encoding the same, and cells, cell lines or other host cells or host organisms that express (and in particularly suitably express, as described herein) or are capable of (suitably) expressing either the first and/or the second fusion protein (and preferably both) form further aspects of the invention.

In particular, an arrangement for performing the methods of the invention may comprise at least the following elements:
a boundary layer that separates a first environment from a second environment;
a binding pair that consists of at least a first binding member and a second binding member, which binding pair is capable of generating a detectable signal;
a translayer protein that is suitably fused or linked (either directly or via a suitable linker or spacer) to one of the binding members of said binding pair (i.e. so as to form a first fusion protein);
a first ligand for the translayer protein that is present in the first environment; and
a second ligand for the translayer protein that is present in the second environment;
   which elements are arranged with respect to each other (and where applicable operably linked to and/or associated with each other) in the manner as further described herein. In particular, the second member of the binding pair may be part of a second fusion protein (which is different from the first fusion protein that comprises the translayer protein and the first binding member of the binding pair), which second fusion protein is as further described herein.

More in particular, an arrangement for performing the methods of the invention may comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a second fusion protein comprising a protein that can bind directly or indirectly to the translayer protein and the other binding member of said binding pair, which second fusion protein is present in the second environment; and
a first ligand for the translayer protein that is present in the first environment;
   which elements are arranged with respect to each other (and where applicable operably linked to and/or associated with each other) in the manner as further described herein.

It should be noted that in the present description and claims, when it is said that a ligand, binding domain, binding unit or other compound or protein “can bind to” another protein or compound, that such binding is most preferably “specific binding” as further defined herein. Also, as further described herein, when a fusion protein is described as “comprising” a first protein, ligand, binding domain, binding member or binding unit and a second protein, ligand, binding domain, binding member or binding unit (and optionally one or more further proteins, ligands, binding domains, binding members or binding units), it should be understood that in such a fusion protein, such proteins, ligands, binding domains, binding members or binding units are suitably linked to each other, either directly or via a suitable spacer or linker.

For the purposes of the present description and claims, a protein (such as a binding domain, binding unit or ligand) is said to bind “directly or indirectly” to the translayer protein if: (i) said protein itself binds (and/or is capable of binding) to the translayer protein (e.g. to an epitope or binding site on the translayer protein, as further described herein); or if (ii) said protein binds (and/or is capable of binding) to a ligand or protein that binds (and/or is capable of binding) to said translayer protein; or if (iii) said protein binds (and/or is capable of binding) to a protein complex that comprises a ligand or protein that binds (and/or is capable of binding) to said translayer protein. In the case of (i), the protein is said herein to bind “directly” to the translayer protein, and in the case of (ii) and (iii), the protein is said herein to bind “indirectly” to the translayer protein. Also, when a protein binds to a protein complex that comprises a ligand or protein that binds to the translayer protein, said protein may bind to said ligand or protein or to any other part, epitope or binding site of said complex).

Thus, in one aspect of the invention, the protein that binds to the translayer protein is chosen from (i) a binding domain, binding unit or other protein that binds (and/or is capable of binding) to an epitope or binding site on the translayer protein; (ii) a binding domain, binding unit or other protein that binds (and/or is capable of binding) to a ligand or protein that binds (and/or is capable of binding) to said translayer protein; and (iii) a binding domain, binding unit or other protein that binds (and/or is capable of binding) to a protein complex that comprises a ligand or protein that binds (and/or is capable of binding) to said translayer protein. In each such case, such a binding domain, binding unit or other protein is preferably as further described herein.

In particular, the protein that binds directly or indirectly to the translayer protein may be chosen from (i) an ISVD that binds (and/or is capable of binding) to an epitope or binding site on the translayer protein; (ii) an ISVD that binds (and/or is capable of binding) to a ligand or protein that binds (and/or is capable of binding) to said translayer protein; and (iii) an ISVD that binds (and/or is capable of binding) to a protein complex that comprises a ligand or protein that binds (and/or is capable of binding) to said translayer protein. Again, in each such case, such an ISVD is preferably as further described herein.

In further aspect of the invention, an arrangement for performing the methods of the invention may comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a second fusion protein comprising a protein that can bind directly (as defined herein) to the translayer protein and the other binding member of said binding pair, which second fusion protein is present in the second environment; and
a first ligand for the translayer protein that is present in the first environment;
   which elements are arranged with respect to each other (and where applicable operably linked to and/or associated with each other) in the manner as further described herein. In this aspect of the invention, the protein that can bind directly (as defined herein) to the translayer protein and that is present in the second fusion protein is preferably a binding domain or binding unit and more preferably an immunoglobulin single variable domain. It should also be understood that in this aspect of the invention, the protein that can bind directly (as defined herein) to the translayer protein and that is present in the second fusion protein acts as the second ligand.

In another aspect of the invention, an arrangement for performing the methods of the invention may comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a first ligand for the translayer protein that is present in the first environment;
a second ligand for the translayer protein, which may optionally be part of a protein complex;
a second fusion protein comprising a protein that can bind indirectly (as defined herein) to the translayer protein and the other binding member of said binding pair, which second fusion protein is present in the second environment;
   which elements are arranged with respect to each other (and where applicable operably linked to and/or associated with each other) in the manner as further described herein. In this aspect of the invention, the second ligand can be any suitable ligand (as further described herein) and the protein that can bind indirectly (as defined herein) to the translayer protein and that is present in the second fusion protein is preferably a binding domain or binding unit and more preferably an immunoglobulin single variable domain. It will also be clear that in this aspect of the invention, the second ligand will not form part of the second fusion protein.

It should be noted that, as further described herein, in the practice of the invention, the first ligand will often be added to the further elements of an already formed/established arrangement of the invention as described herein, and that consequently arrangements of the invention without the first ligand being present (i.e. before the first ligand is added) form further aspects of the invention (as do methods in which a first ligand is added to an arrangement of the invention in which said first ligand is not or not yet present).

In the present description and claims, the term “second ligand” is used to denote the ligand, binding domain, binding unit or other chemical entity that, in the methods and arrangements described herein, binds directly to the translayer protein or is capable of binding directly to the translayer protein (or forms part of a protein complex that binds directly to the translayer protein or that is capable of binding directly to the translayer protein).

As will be clear from the further description herein, said second ligand can either be part of the second fusion protein or it can be separate from the second fusion protein. In either case (i.e. irrespective of whether the second ligand is part of the second fusion protein or not), the second ligand is preferably such that it is capable of binding to a conformational epitope on the translayer protein (or such that it can form part of a protein complex that binds directly to the translayer protein or that is capable of binding directly to the translayer protein). More preferably, the second ligand (and/or the protein complex that comprises the second ligand) is preferably such that it specifically binds to one or more functional, active and/or druggable conformations of the translayer protein, that it induces the formation of and/or stabilizes one or more functional, active and/or druggable conformations of the translayer protein (and/or shifts the conformational equilibrium of the translayer protein towards one or more such conformations); and/or that it induces the formation of and/or stabilizes a complex of the translayer protein, the first ligand and the second ligand.

When the second ligand is part of the second fusion protein, it can be any ligand, binding domain, binding unit, peptide, protein or other chemical entity that can bind directly to the translayer protein and that can suitably be included in the second fusion protein. Preferably, as further described herein, when it is part of the second fusion protein, the second ligand will be a suitable binding domain or binding unit, and in particular an immunoglobulin single variable domain.

When the second ligand is separate from the second fusion protein, it can be any ligand or protein that can bind directly to the translayer protein and/or that can form part of a protein complex that can bind to the translayer protein. For example, as further described herein, such a second ligand may be a naturally occurring ligand of the translayer protein (such as a naturally occurring G-protein, for example the G-protein that naturally occurs in the cell or cell line used), a semi-synthetic or synthetic analog or derivative of such a naturally occurring ligand or an ortholog of such a naturally occurring ligand (such as a “chimeric” G-protein as described herein). Also, when the second ligand is not part of the second fusion protein, the second fusion protein will comprise a binding domain or binding unit that can bind indirectly (as defined herein) to the translayer protein, i.e. a binding domain or binding unit that can bind to the second ligand and/or to a protein complex that comprises the second ligand. Again, as also further described herein, such a binding domain or binding unit may in particular be an immunoglobulin single variable domain, such as a camelid-derived ISVD (or it may comprise one or more such immunoglobulin single variable domains, such as two or three such immunoglobulin single variable domains, which may be the same or different, as further described herein).

As further described herein, in one aspect of the invention, the arrangement of the invention can be present in a suitable cell or cell line and/or the methods of the invention can be performed using a suitable cell or cell line that suitably contains an (operable) arrangement of the invention.

Thus, as further described herein, the invention also relates to a cell or cell line that suitably contains an arrangement of the invention and/or that suitably expresses (as defined herein) or is capable of suitably expressing the elements of an arrangement of the invention so as to provide an arrangement of the invention (and in particular an arrangement of the invention that is operable in said cell or cell line). The invention also relates to a cell or cell line that comprises and/or that suitably expresses (as defined herein) or is capable of suitably expressing a first fusion protein as described herein. The invention also relates to a cell or cell line that comprises and/or that suitably expresses or is capable of suitably expressing a second fusion protein as described herein. In yet another aspect, the invention relates to a cell or cell line that comprises and/or that suitably expresses or is capable of suitably expressing both a first fusion protein as described herein and a second fusion protein as described herein. In aspects and embodiments where the second ligand does not form part of the second fusion protein, such cells or cell lines may also contain or suitably express a suitable second ligand.

As also described herein, in one aspect of the invention, the arrangement of the invention can be present in a suitable liposome or vesicle and/or the methods of the invention can be performed using a liposome or vesicle that suitably contains an (operable) arrangement of the invention.

Thus, as further described herein, the invention also relates to a liposome or vesicle that suitably contains (the elements of) an arrangement of the invention, in particular so as to provide an arrangement of the invention that is operable in said liposome or vesicle. The invention also relates to a liposome or vesicle that comprises a first fusion protein as described herein. The invention also relates to liposome or vesicle a cell or cell line that comprises a second fusion protein as described herein. In yet another aspect, the invention relates to a liposome or vesicle that comprises both a first fusion protein as described herein and a second fusion protein as described herein. In aspects and embodiments where the second ligand does not form part of the second fusion protein, such a liposome or vesicle may also contain a suitable second ligand.

Thus, as further described herein and as will be illustrated by means of the appended non-limiting Figures, and depending on whether the second ligand is part of the second fusion protein or not, the invention envisages at least three preferred embodiments of the methods and arrangements of the invention.

In a first such preferred embodiment (schematically shown in FIG. 1), the second binding member of the binding pair will be suitably fused or linked (either directly or via a suitable linker or spacer) to the second ligand. According to this preferred embodiment, an arrangement for performing the methods of the invention may thus comprise at least the following elements:
a boundary layer that separates a first environment from a second environment;
a binding pair that consists of at least a first binding member and a second binding member, which binding pair is capable of generating a detectable signal;
a translayer protein that is suitably fused or linked (either directly or via a suitable linker or spacer) to one of the binding members of said binding pair;
a first ligand for the translayer protein that is present in the first environment; and
a second ligand for the translayer protein that is present in the second environment and that is suitably fused or linked (either directly or via a suitable linker or spacer) to the other binding member of said binding pair;
   which elements are arranged with respect to each other (and where applicable operably linked to and/or associated with each other) in the manner as further described herein.

In particular, as further described herein, such an arrangement may comprise the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a first ligand for the translayer protein that is present in the first environment; and
a second fusion protein comprising a second ligand for the translayer protein and the other binding member of said binding pair, which second fusion protein is present in the second environment;
   which elements are arranged with respect to each other (and where applicable operably linked to and/or associated with each other) in the manner as further described herein.

It will be clear to the skilled person that in this first embodiment, the “second ligand” will be a binding domain, binding unit or other protein that binds (and/or is capable of binding) directly to an epitope or binding site on the translayer protein. Again, said binding domain or binding unit is preferably an immunoglobulin single variable domain as further described herein.

In a second such preferred embodiment (schematically shown in FIG. 2), the second binding member of the binding pair will be suitably fused or linked (either directly or via a suitable linker or spacer) to a binding domain or binding unit that does not bind directly to the translayer protein, but instead binds to the second ligand (which in turn can bind to the translayer protein). According to this preferred embodiment, an arrangement for performing the methods of the invention may thus comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a first ligand for the translayer protein that is present in the first environment;
a second ligand for the translayer protein that is present in the second environment; and
a second fusion protein that is present in the second environment and that comprises a binding domain or binding unit that can bind to the second ligand, which binding domain or binding unit is suitably fused or linked (either directly or via a suitable linker or spacer) to the other binding member of said binding pair;
   which elements are arranged with respect to each other (and where applicable operably linked to and/or associated with each other) in the manner as further described herein.

It will be clear to the skilled person that in this second embodiment, the binding domain or binding unit that is present in the second fusion protein will bind “indirectly” to the translayer protein, i.e. by binding to the second ligand which binds to the translayer protein. Again, said binding domain or binding unit is preferably (and/or preferably essentially consists of) an immunoglobulin single variable domain as further described herein. The binding domain or binding unit may also comprise or essentially consist of two or more immunoglobulin single variable domains (such as two or three immunoglobulin single variable domains), which are each capable of (specifically) binding to the second ligand (i.e. to the same epitope or binding site on the second ligand or to different epitopes/binding sites on the second ligand) and which may be the same or different (as further described herein), and which are suitably linked or fused to each other and to the other binding member of said binding pair (optionally via suitable linkers or spacers) so as to form a second fusion protein that is suitable for use in the invention. For example and without limitation, such a binding domain or binding unit may comprise two or three copies of the ConfoBody CA4437 (SEQ ID NO:4 in WO2012/75643 and SEQ ID NO:2 herein), which are suitably linked or fused to each other and to the other binding member of said binding pair (optionally via suitable linkers or spacers) so as to form a second fusion protein that is suitable for use in the invention. Also, in this embodiment, the second ligand can be any suitable ligand for the translayer protein as further described herein. Also, again, such a “multivalent” binding domain comprising two or more ISVDs should most preferably be such that its binding to the second ligand essentially does not interfere with the ability of the second ligand to bind to the translayer protein and/or to form (or facilitate the formation of) a complex between the second ligand, the translayer protein and the first ligand.

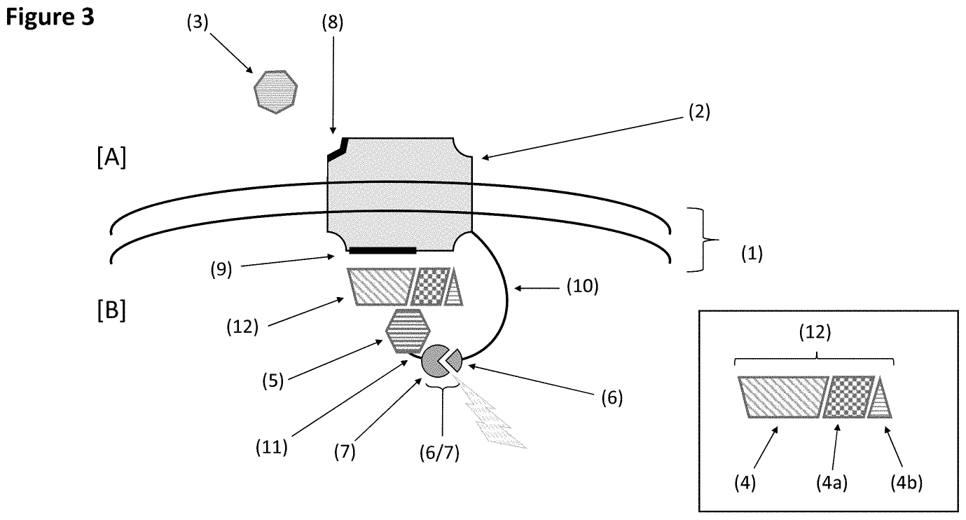

In a third preferred embodiment (schematically shown in FIG. 3), the second binding member of the binding pair will be suitably fused or linked (either directly or via a suitable linker or spacer) to a binding domain or binding unit that does not bind directly to the translayer protein, but instead binds to a protein complex that comprises at least the second ligand for the translayer protein (which protein complex may either bind to, or be bound by, the translayer protein and/or comprise the translayer protein). According to this preferred embodiment, an arrangement for performing the methods of the invention may thus comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a first ligand for the translayer protein that is present in the first environment;
a protein complex that at least comprises a second ligand for the translayer protein, which protein complex is present in the second environment; and
a second fusion protein that is present in the second environment and that comprises a binding domain or binding unit that can bind to the protein complex, which binding domain or binding unit is suitably fused or linked (either directly or via a suitable linker or spacer) to the other binding member of said binding pair;
   which elements are arranged with respect to each other (and where applicable operably linked to and/or associated with each other) in the manner as further described herein.

It will be clear to the skilled person that in this third embodiment, the binding domain or binding unit that is present in the second fusion protein will bind “indirectly” to the translayer protein, i.e. by binding to a protein complex that comprises the second ligand. Again, said binding domain or binding unit is preferably an immunoglobulin single variable domain as further described herein, and the second ligand can be any suitable ligand for the translayer protein that can be part of a protein complex as further described herein.

Also, said binding domain or binding unit in the second fusion protein may comprise two or more immunoglobulin single variable domains, each of which is capable of binding to a different epitope, part, domain or subunit on/of said protein complex, such as to two different epitopes on the G-protein complex. For example and without limitation, when the protein complex is a heterotrimeric G-protein, said binding domain or binding unit may comprise two or three different ISVDs, in which each ISVD is capable of (specifically) binding to a different subunit of said G-protein (in which preferably, at least one of said ISVDs is capable of specifically binding to the G-alpha subunit that is present in said heterotrimeric G-protein). A specific but non-limiting example of such a binding domain or binding unit may for example comprise the ConfoBodies CA4435 (SEQ ID NO:1 in WO2012/75643 and SEQ ID NO:1 herein) and CA4437 (SEQ ID NO:4 in WO2012/75643 and SEQ ID NO:2 herein), which are suitably linked or fused to each other and to the other binding member of said binding pair (optionally via suitable linkers or spacers) so as to form a second fusion protein that is suitable for use in the invention.

The use of such “multivalent” binding domains or binding units (i.e. comprising two or more ISVDs) in the second fusion protein may also lead to improved sensitivity in the assays described herein compared to the use of the corresponding ISVD(s) in a monovalent format (i.e. comprising only one of said ISVDs).

More generally, the arrangements for performing the methods of the invention in their various aspects and embodiments will usually, and preferably, at least comprise at least the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a first ligand for the translayer protein that is present in the first environment;
a second ligand for the translayer protein that is present in the second environment; and
a second fusion protein that comprises the other binding member of said binding pair (i.e. such that said other member of the binding pair is also present in the second environment);
   which elements are arranged with respect to each other (and where applicable operably linked to and/or associated with each other) in the manner as further described herein. In particular:
in the first preferred embodiment described herein, the second fusion protein will comprise the other binding member of said binding pair and the second ligand;
in the second preferred embodiment described herein, the second fusion protein will comprise the other binding member of said binding pair and a binding domain or binding unit that can bind to the second ligand; and
in the third preferred embodiment described herein, the second fusion protein will comprise the other binding member of said binding pair and a binding domain or binding unit that can bind to a protein complex that comprises at least the second ligand.

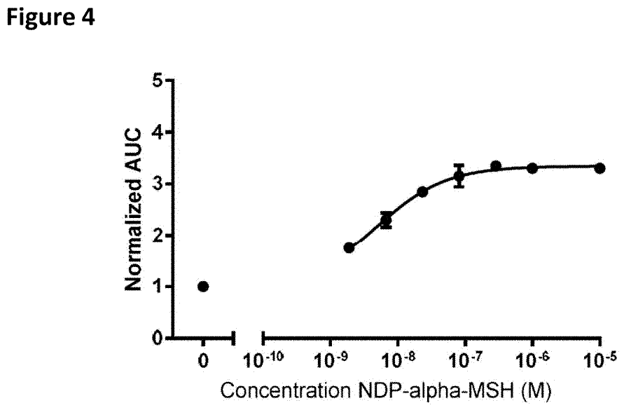

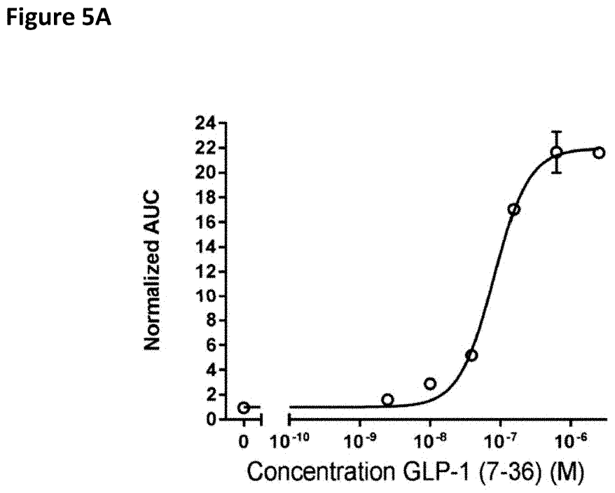

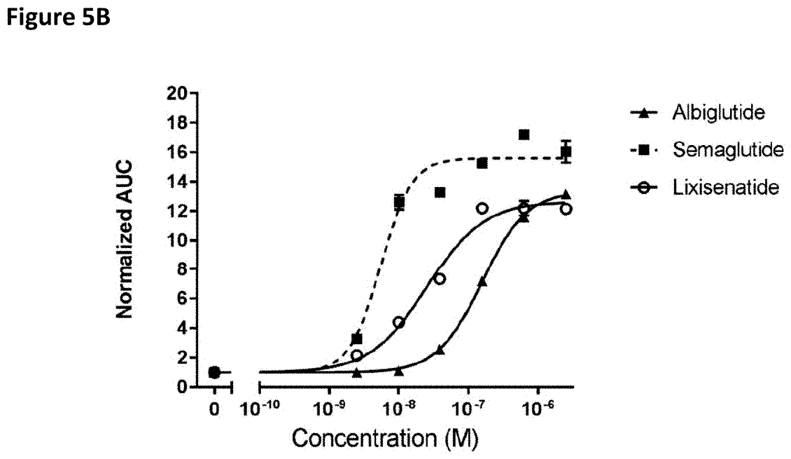

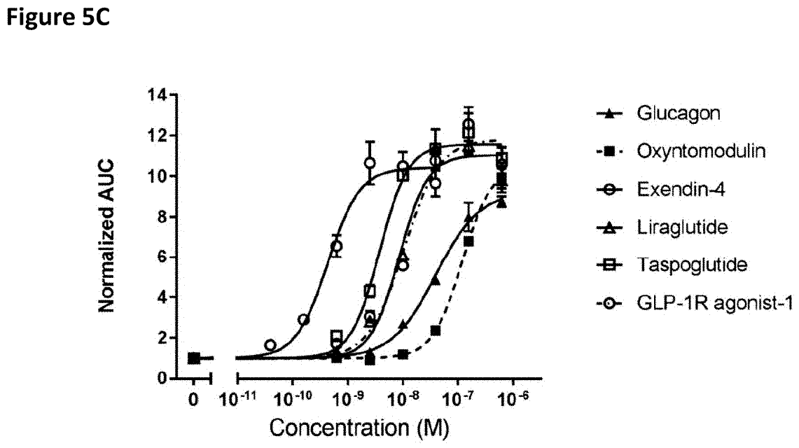

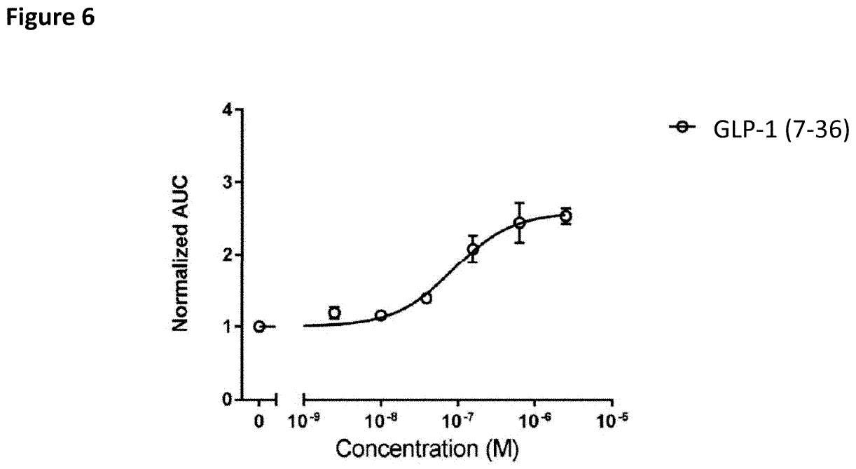

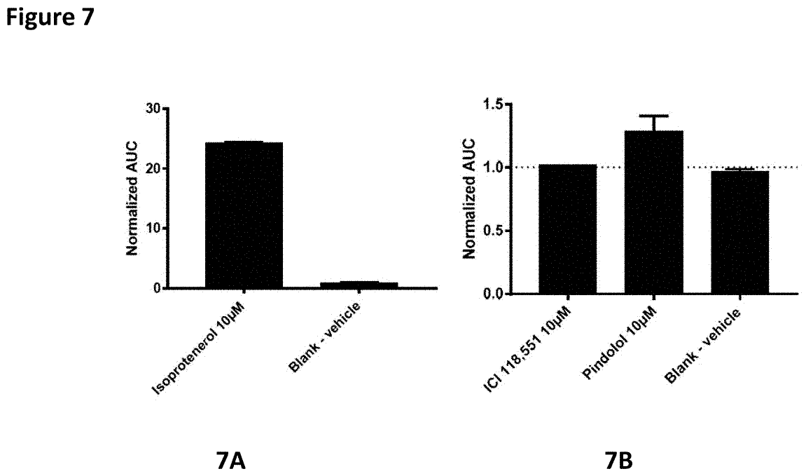

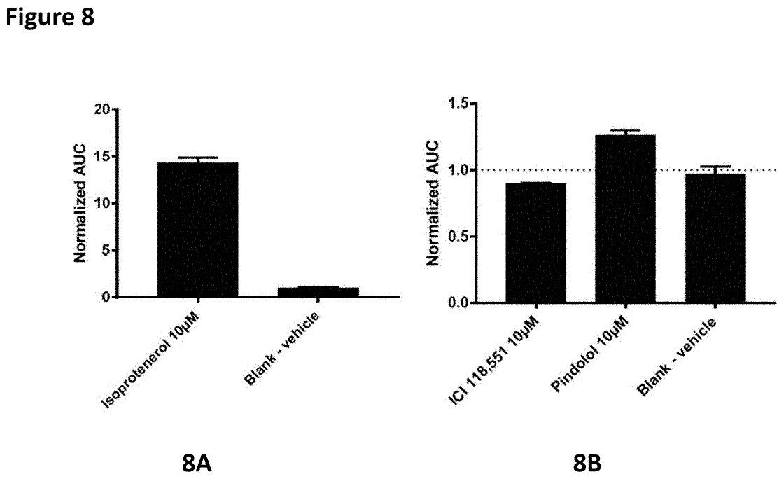

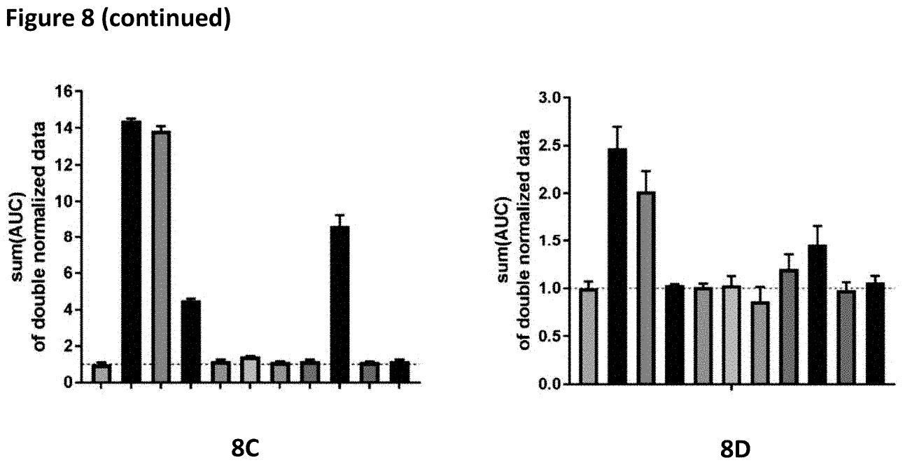

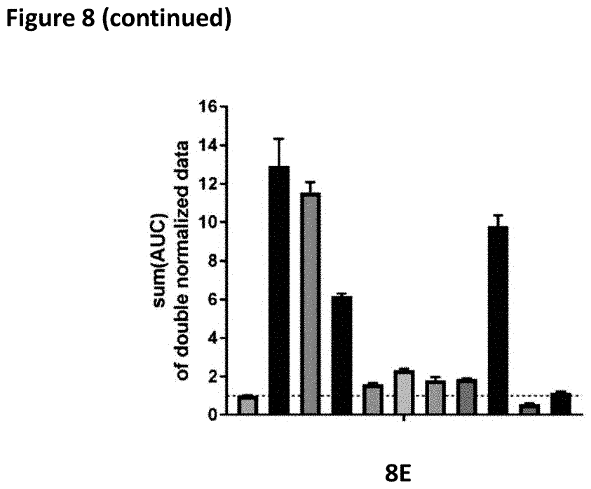

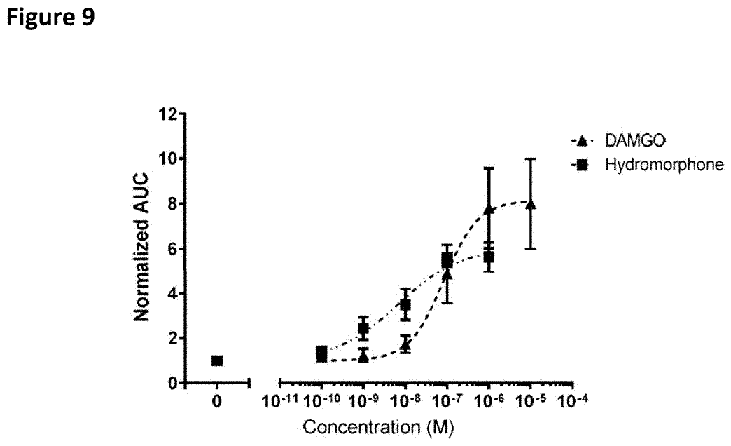

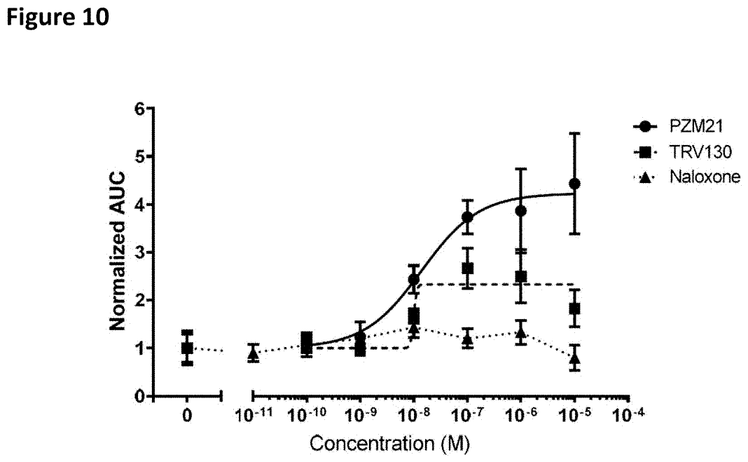

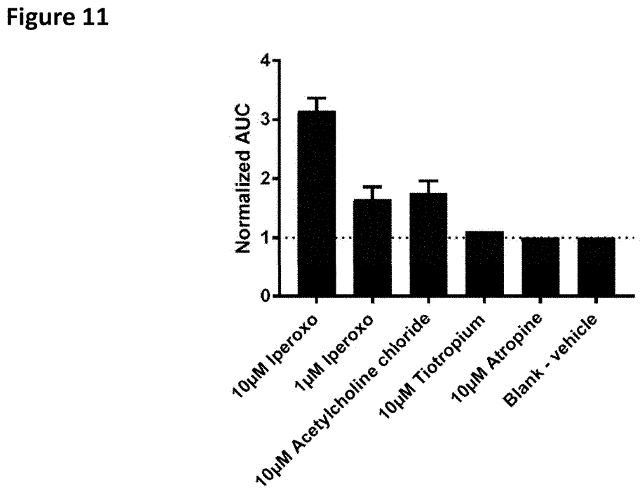

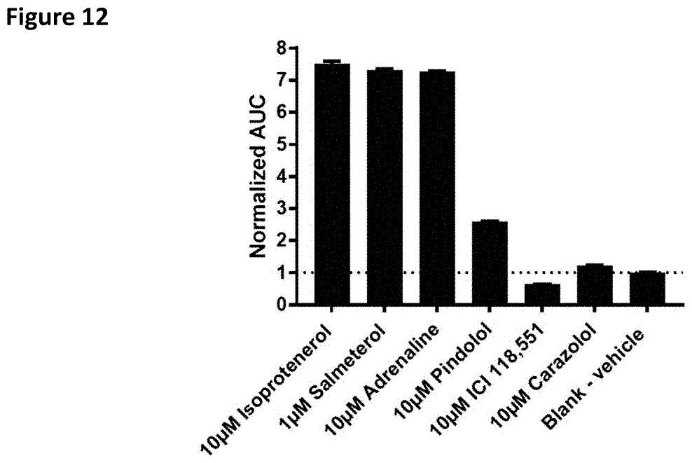

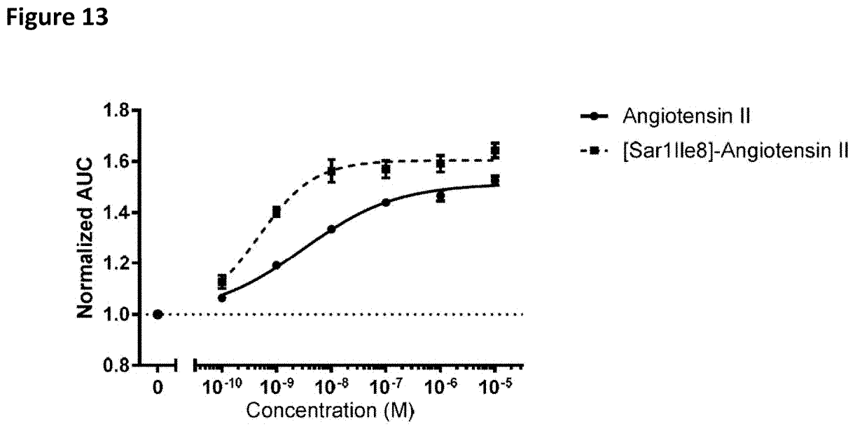

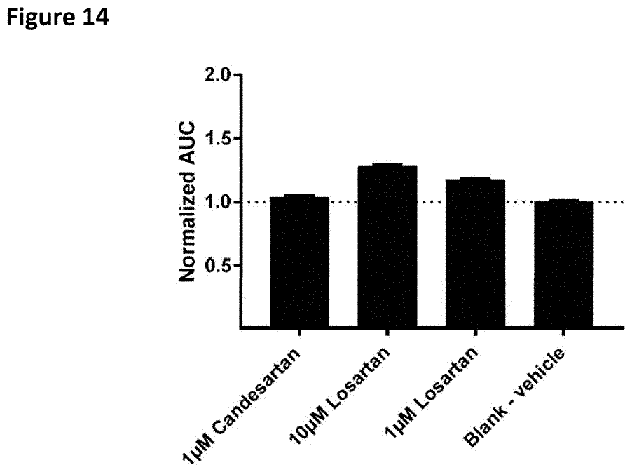

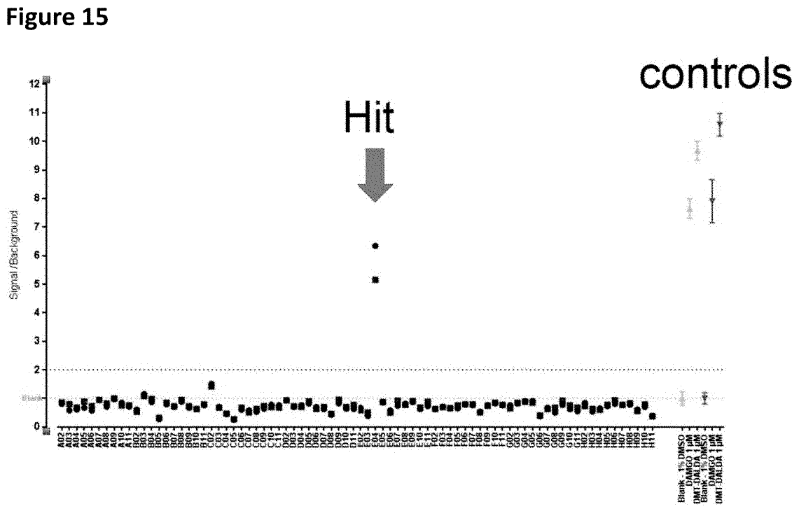

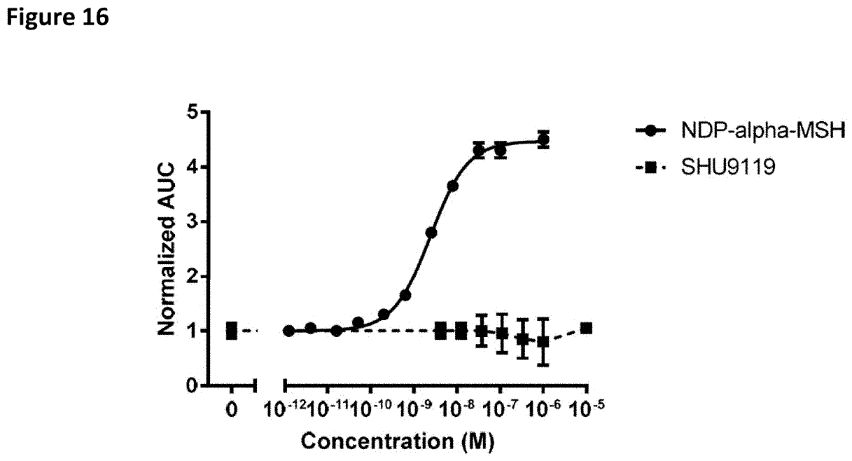

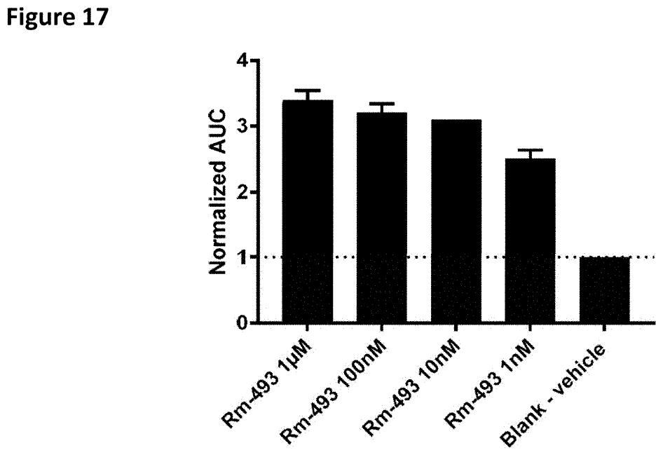

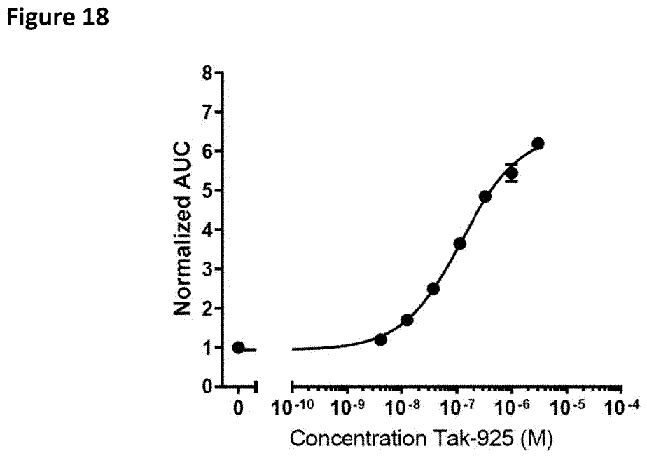

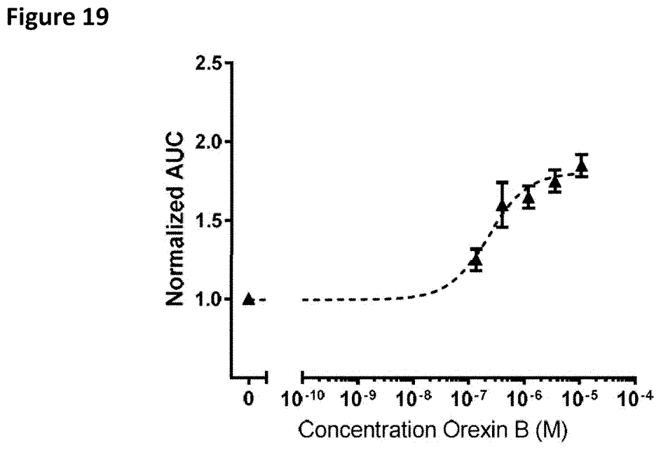

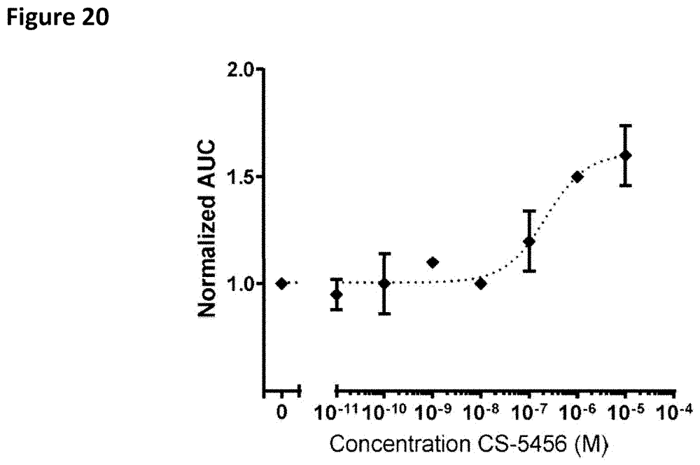

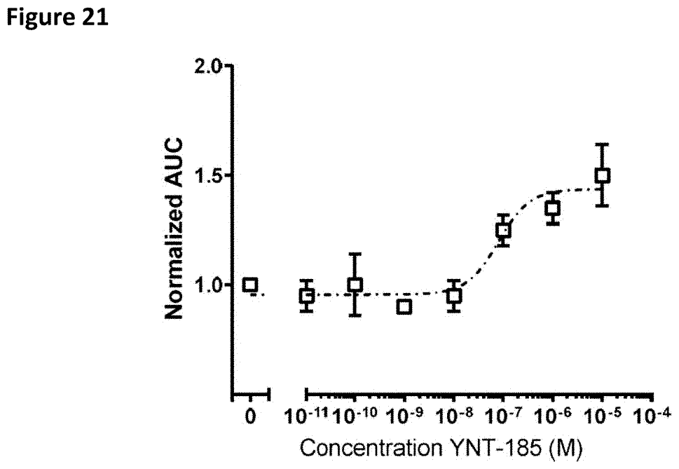

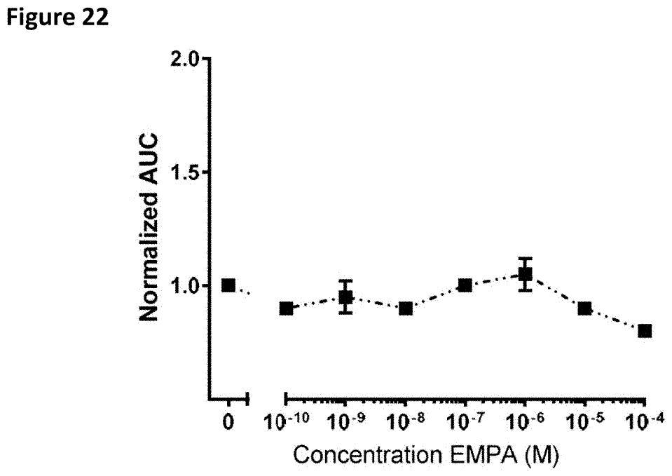

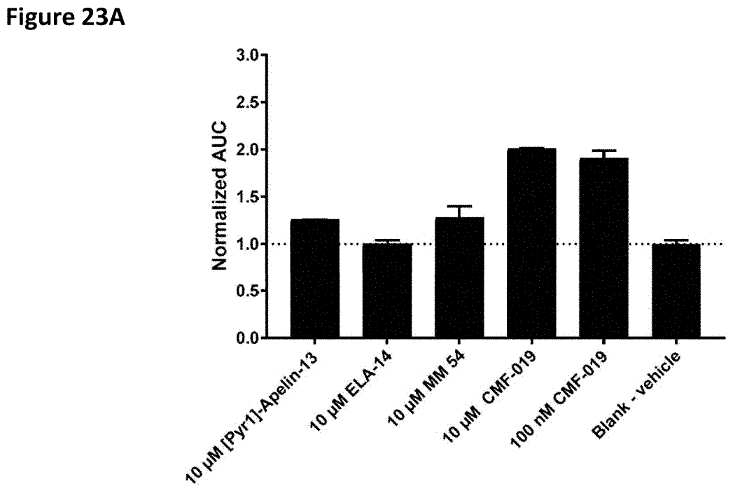

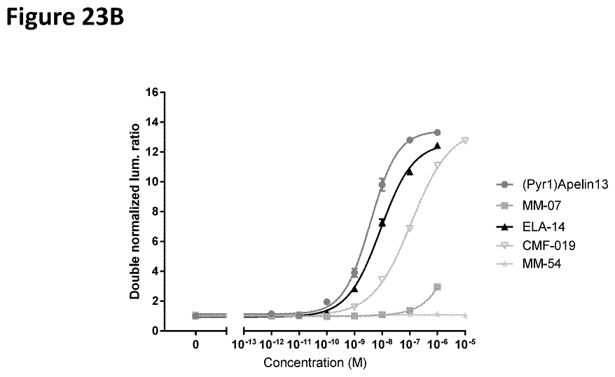

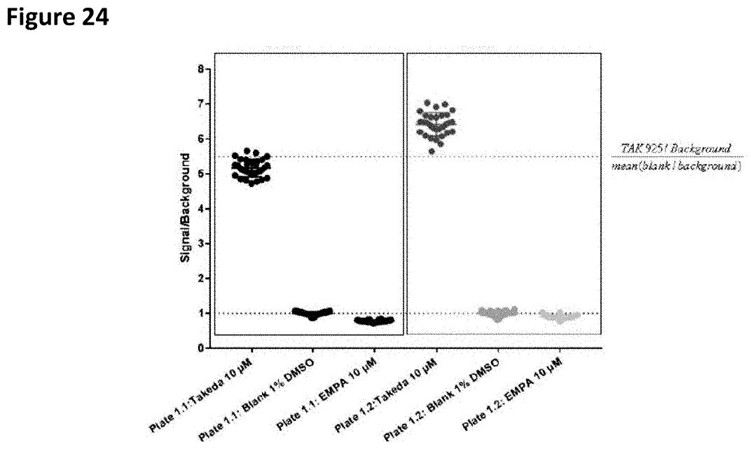

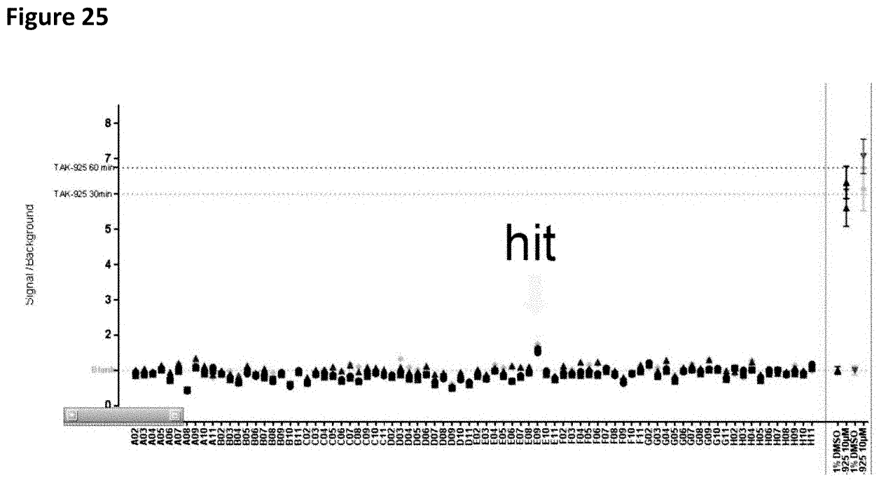

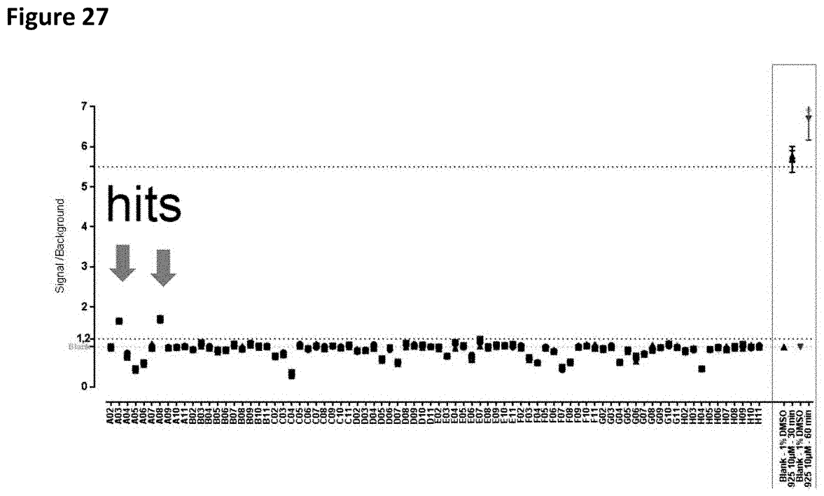

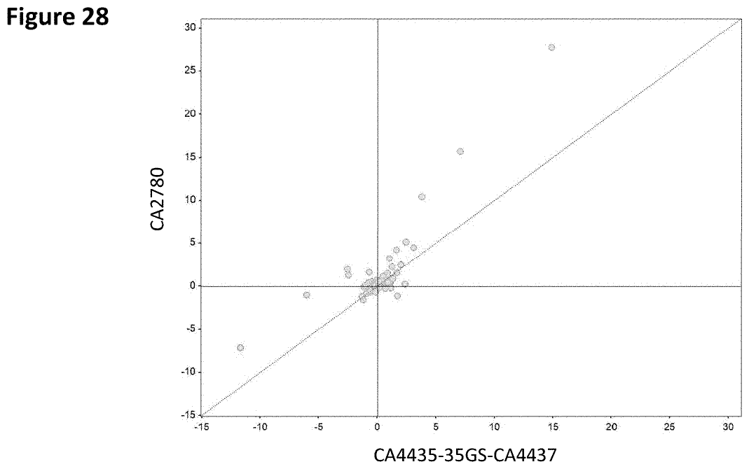

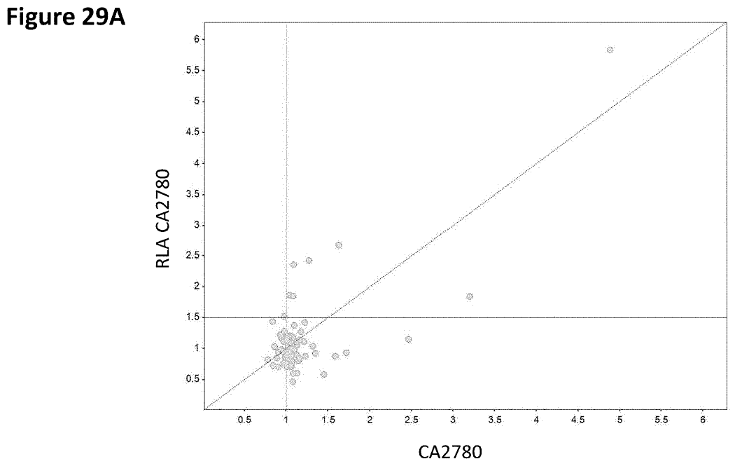

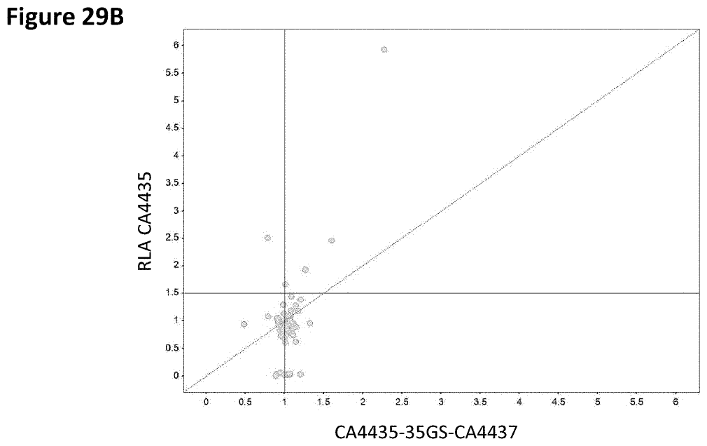

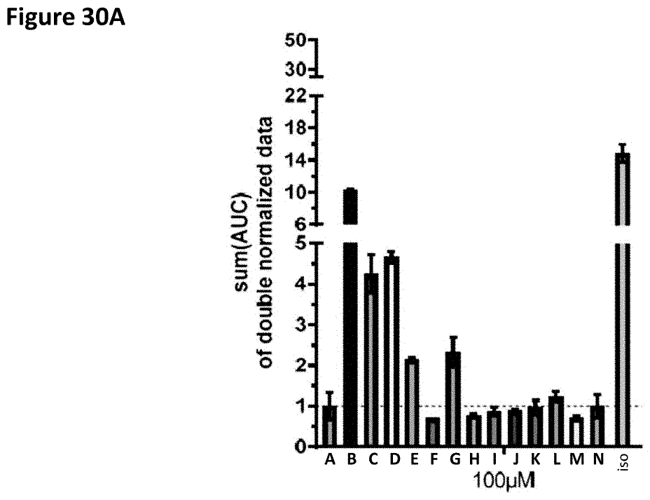

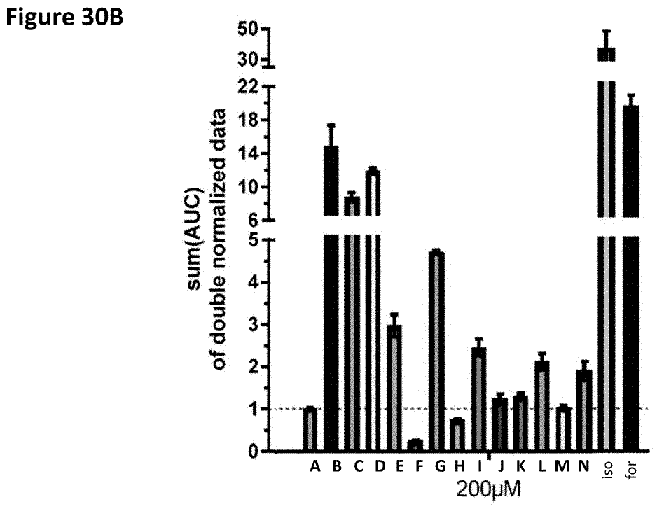

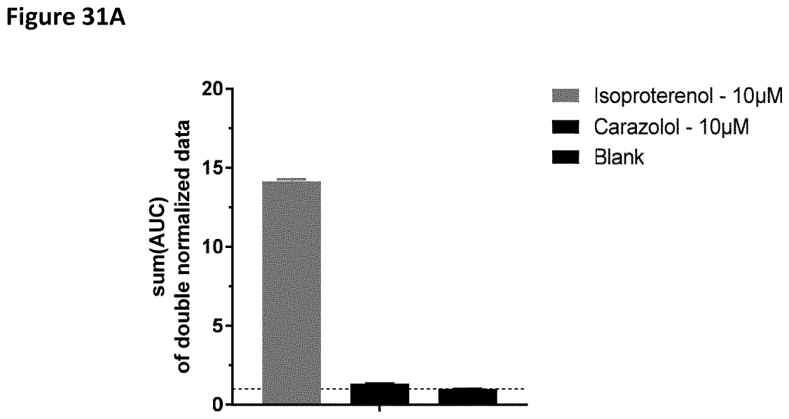

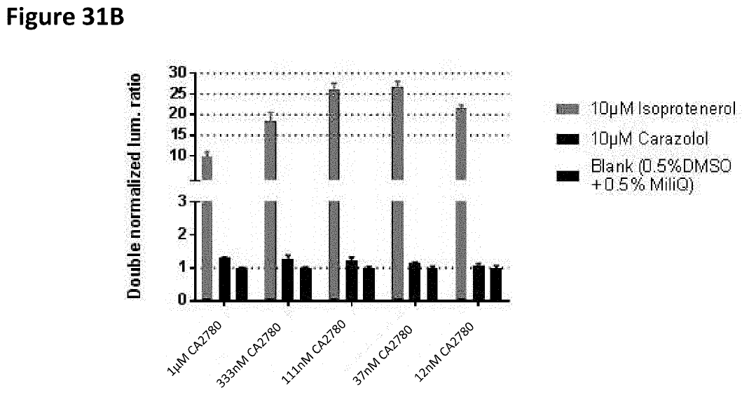

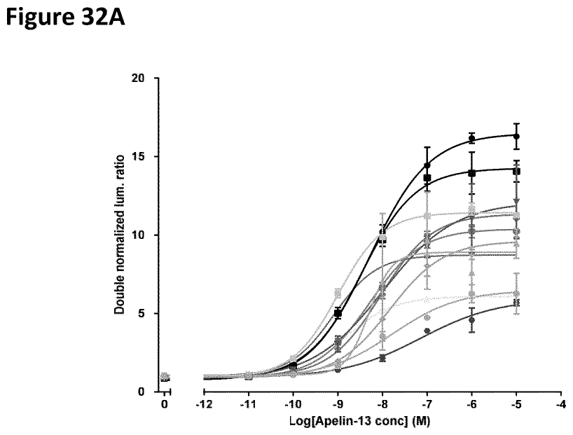

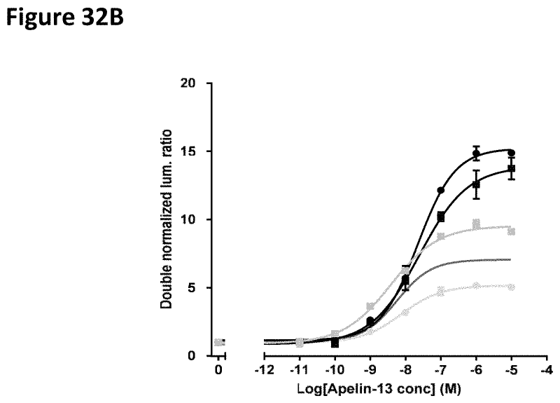

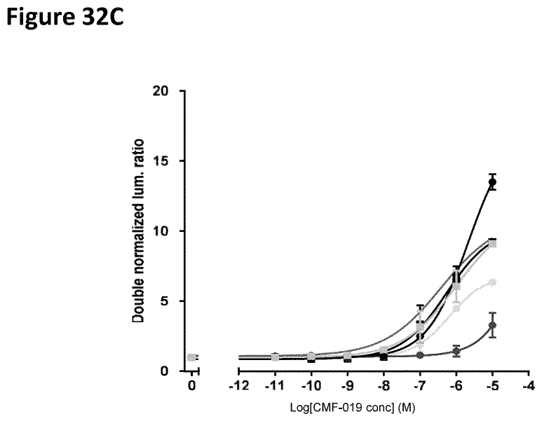

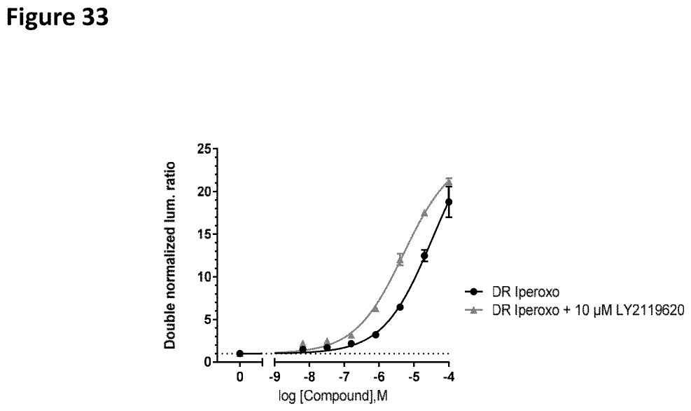

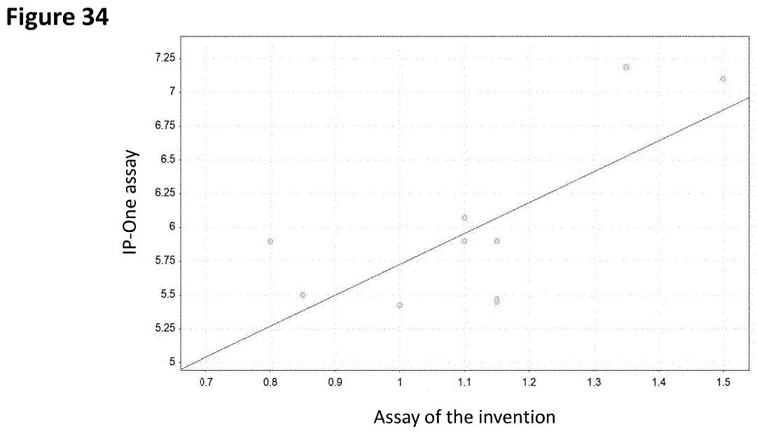

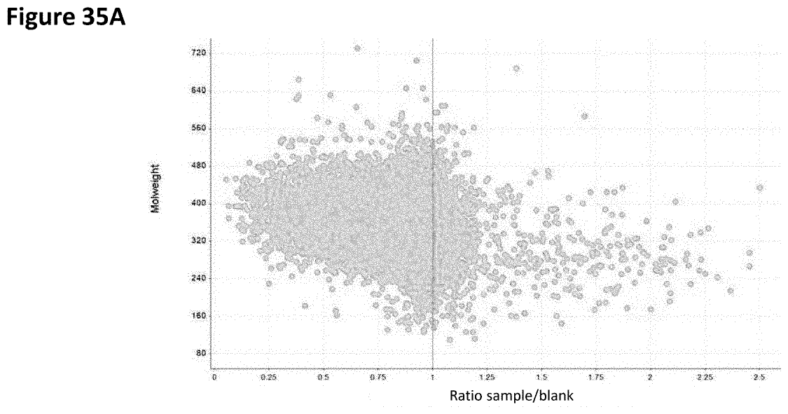

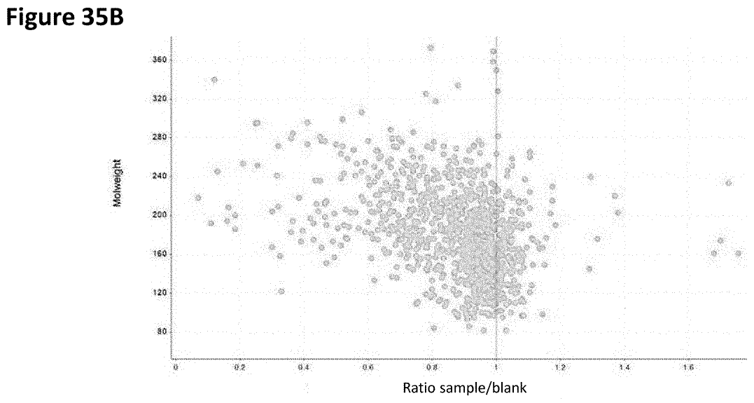

The invention will now be illustrated by means of the further description herein, the Experimental Part below, and the appended non-limiting Figures. In the Figures:
a) FIG. 1 schematically shows a first arrangement of the invention, in which the second ligand (indicated as (4) in FIG. 1), forms part of the second fusion protein (which in the embodiment shown in FIG. 1 is formed by the second ligand (4), the linker (11) and the second member (7) of the binding pair (6/7)) binds directly (as defined herein) to the translayer protein (2). In the set-up shown in FIG. 1:
   the boundary layer is indicated as (1);
   the first environment is indicated as [A];
   the second environment is indicated as [B];
   the translayer protein is indicated as (2);
   the first ligand is indicated as (3);
   the first binding site on the translayer protein (2) that is exposed to the first environment [A] and to which the first ligand (3) can bind is indicated as (8);
   the second ligand is indicated as (4);
   the second binding site on the translayer protein (2) that is exposed to the second environment [B] and to which the second ligand (4) can bind is indicated as (9);
   the binding pair that can generate a detectable signal is indicated as (6/7) and consists of a first binding member (6) linked to the translayer protein (2) (either directly of via linker or spacer (10)) and a second binding member (7) linked to the second ligand (4) (either directly of via linker or spacer (11));
   the first fusion protein comprises the translayer protein (2) that is fused, either directly or via the linker (10), to the first binding member (6);
   the second fusion protein comprises the second ligand (4) that is fused, either directly or via the linker (11), to the second binding member (7); and
   the first and second fusion proteins are arranged with respect to each other and with respect to the boundary layer (1) in such a way that, when the second ligand (4) binds to the translayer protein (2) (i.e. directly via the binding site (9)), the first binding member (6) and the second binding member (7) can come into contact or close proximity with/to each other (or otherwise suitably associate) so as to generate a detectable signal (indicated with the flash symbol in FIG. 1).
b) FIG. 2 schematically shows a second arrangement of the invention, in which the second ligand (indicated as (4) in FIG. 2) is separate from the second fusion protein (which in the embodiment shown in FIG. 2 is formed by the binding domain (5), the linker (11) and the second member (7) of the binding pair (6/7)) and in which the binding domain (5) which is present in the second fusion protein binds indirectly (as defined herein, and in the case of FIG. 2 via the second ligand (4)) to the translayer protein (2). In the set-up shown in FIG. 2:
   the boundary layer is indicated as (1);
   the first environment is indicated as [A];
   the second environment is indicated as [B];
   the translayer protein is indicated as (2);
   the first ligand is indicated as (3);
   the first binding site on the translayer protein (2) that is exposed to the first environment [A] and to which the first ligand (3) can bind is indicated as (8);
   the second ligand is indicated as (4);
   the second binding site on the translayer protein (2) that is exposed to the second environment [B] and to which the second ligand (4) can bind is indicated as (9);
   the binding domain or binding unit that can bind to the second ligand (4) is indicated as (5);
   the binding pair that can generate a detectable signal is indicated as (6/7) and consists of a first binding member (6) linked to the translayer protein (2) (either directly of via linker or spacer (10)) and a second binding member (7) linked to the binding domain or binding unit (5) (either directly of via linker or spacer (11));
   the first fusion protein comprises the translayer protein (2) that is fused, either directly or via the linker (10), to the first binding member (6);
   the second fusion protein comprises the binding domain (5) that is fused, either directly or via the linker (11), to the second binding member (7); and
   the first and second fusion proteins are arranged with respect to each other and with respect to the boundary layer (1) in such a way that, when the binding domain (5) binds to the translayer protein (2) (i.e. indirectly by binding to the second ligand (4) which in turn binds to the translayer protein (2) via the binding site (9)), the first binding member (6) and the second binding member (7) can come into contact or close proximity with/to each other (or otherwise suitably associate) so as to generate a detectable signal (indicated with the flash symbol in FIG. 2).
c) FIG. 3 schematically shows a third arrangement of the invention, in which the second ligand (indicated as (4) in FIG. 3) is separate from the second fusion protein and forms part of a protein complex (12) that is formed by the second ligand (4) and one or more further proteins (in the case of FIG. 3, for illustration purposes, said complex is exemplified as comprising the second ligand (4) and two further proteins (4a) and (4b)—see also the insert in FIG. 3). In the embodiment shown in FIG. 3, the second ligand (4) is again separate from the second fusion protein (which in the embodiment shown in FIG. 3 is formed by the binding domain (5), the linker (11) and the second member (7) of the binding pair (6/7)) and the binding domain (5) that is present in the second fusion protein binds indirectly (as defined herein, and in the case of FIG. 3 via the protein complex (12)) to the translayer protein (2). In the set-up shown in FIG. 3:
   the boundary layer is indicated as (1);
   the first environment is indicated as [A];
   the second environment is indicated as [B];
   the translayer protein is indicated as (2);
   the first ligand is indicated as (3);
   the first binding site on the translayer protein (2) that is exposed to the first environment [A] and to which the first ligand (3) can bind is indicated as (8);
   the second ligand is indicated as (4), and forms a complex (12) with one or more other proteins (for illustration purposes, in FIG. 3, the complex (12) is represented as a complex comprising three proteins/subunits, namely the second ligand (4) and two further subunits (4a) and (4b)—see also the insert in FIG. 3);
   the second binding site on the translayer protein (2) that is exposed to the second environment [B] and to which the complex (12) (4) can bind is indicated as (9);
   the binding domain or binding unit that can bind to complex (12) is indicated as (5);
   the binding pair that can generate a detectable signal is indicated as (6/7) and consists of a first binding member (6) linked to the translayer protein (2) (either directly of via linker or spacer (10)) and a second binding member (7) linked to the binding domain or binding unit (5) (either directly of via linker or spacer (11));
   the first fusion protein comprises the translayer protein (2) that is fused, either directly or via the linker (10), to the first binding member (6);
   the second fusion protein comprises the binding domain (5) that is fused, either directly or via the linker (11), to the second binding member (7); and
   the first and second fusion proteins are arranged with respect to each other and with respect to the boundary layer (1) in such a way that, when the binding domain (5) binds to the translayer protein (2) (i.e. indirectly by binding to the complex (12) which in turn binds to the translayer protein (2) via the binding site (9)), the first binding member (6) and the second binding member (7) can come into contact or close proximity with/to each other (or otherwise suitably associate) so as to generate a detectable signal (indicated with the flash symbol in FIG. 3).
d) FIG. 4 is a graph showing the dose response curve for NDP-alpha-MSH obtained using the MC4R screening assay with CA4437 described in Example 1;
e) FIGS. 5A to 5C are graphs showing the dose response curves for the indicated compounds obtained using the GLP-1R screening assay with CA4437 described in Example 2;
f) FIG. 6 is a graph showing the dose response curves for GLP-1 (7-36) amide obtained using the GLP-1R screening assay with CA4435 described in Example 2;
g) FIGS. 7A and 7B are graphs showing assay results obtained using the Beta-2-AR screening assay with CA4437 described in Example 3;
h) FIG. 8A to 8E are graphs showing the assay results obtained using the Beta-2-AR screening assay with CA4435 (FIGS. 8A, 8B, 8D), CA4437 (FIG. 8C) and a CA4435-35GS-CA4437 fusion (FIG. 8E) described in Example 3;
i) FIGS. 9 and 10 are graphs showing the dose response curves for the indicated compounds obtained using the MOR screening assay described in Example 4;
j) FIG. 11 is a graph showing assay results obtained using the M2R screening assay described in Example 5;
k) FIG. 12 is a graph showing assay results obtained using the Beta-2AR screening assay described in Example 6;
1) FIG. 13 is a graph showing the dose response curves for the indicated compounds obtained using the AT1R screening assay described in Example 7;
m) FIG. 14 is a graph showing assay results obtained using the AT1R screening assay described in Example 7;
n) FIG. 15 shows the results from the compound library screening performed in Example 8;
o) FIG. 16 is a graph showing the dose response curves for the indicated compounds obtained using the recombinant MC4R screening assay described in Example 9;
p) FIG. 17 is a graph showing assay results obtained using the recombinant MC4R screening assay described in Example 9;
q) FIGS. 18 to 22 are graphs showing the dose response curves for the indicated compounds obtained using the recombinant OX2R screening assay described in Example 10;
r) FIGS. 23A and B are graphs showing assay results obtained using the two recombinant APJ receptor screening assays described in Example 11. FIG. 23A shows the results obtained with a recombinant apelin receptor having the ICLs of a mu-opioid receptor (MOR) and FIG. 23B shows the results obtained with a recombinant apelin receptor having the ICLs from a beta-2AR receptor;
s) FIGS. 24 to 27 shows the results from the compound library screening performed in Example 12.
t) FIG. 28 is a plot of screening results obtained in Example 14 for a collection of 78 compound fragments when tested using two assays of the invention (both using a Beta-2AR-LgBiT fusion, but with one assay using an CA2780-SmBiT fusion and one assay using a CA4435-35GS-CA4437-LgBiT fusion). In FIG. 28, the x-axis represents the results obtained in the assay using the CA4435-35GS-CA4437-LgBiT fusion, the y-axis represents the results obtained in the assay using the CA2780-SmBiT fusion, and each dot represents the result obtained in both assays for one of the 78 compounds tested.
u) FIGS. 29A and 29B are plot of screening results obtained in Example 15 for a collection of compound fragments when tested using a radioligand assay and a corresponding assay of the invention). In FIGS. 29A and B, the x-axis represents the results obtained using the assay of the invention, the y-axis represents the results obtained with the radioligand assay in the assay, each dot represents the result obtained for one of the compounds when tested in both the radioligand assay and the assay of the invention.
v) FIGS. 30A and 30B are graphs showing the results of the testing (at 100 μM and 200 μM, respectively) of the compounds referred to in Example 16 and Table 3 in a GloSensor cAMP assay for Beta-2AR;
w) FIGS. 31A and 31B are graphs showing a comparison of the results obtained in Example 17 when a cell-based assay of the invention was compared to a comparable membrane-based assay of the invention;
x) FIGS. 32A to C show dose-response curves to apelin obtained with different VHH's (Example 18).
y) FIG. 33 shows dose-response curves generated in Example 19 for iperoxo against the M2 receptor using an assay of the invention, with and without the presence of LY2119620 (an allosteric modulator of the M2 receptor).
z) FIG. 34 is a plot obtained in Example 20 comparing results from an OX2 assay of the invention (using a recombinant OX2 fusion) and an OX2 IP-One assay, with the x-axis representing the data obtained in the assay of the invention, the y-axis representing the data obtained in the IP-One assay and each dot representing the results for a single compound.
aa) FIGS. 35A and 35B are plots obtained in Example 21 when a large compound library was screened against a recombinant OX2 receptor using an assay of the invention. FIG. 35A shows the results obtained when the compounds were tested at 30 μM) and FIG. 35B shows the results obtained when the compounds tested at 200 μM), with the x-axis representing the ratio of the signal obtained with the compound tested (“sample”) vs signal given by the carrier solvent (“blank”) and each dot representing the result obtained for a single compound.

From the Figures and the further description herein, it will be clear to the skilled person that some elements of the arrangements of the invention (such as the boundary layer, the translayer protein, the binding pair, any linkers and the first ligand) will be present in the various aspects and embodiments of the invention as contemplated herein. Thus, when a detailed description is given herein of any such element (including any preferences for any such element), it should be understood that such description applies to all aspects and embodiments of the invention in which such element is present or used, unless explicitly stated otherwise herein.

In the methods and arrangements of the invention, the boundary layer (1) can be any layer (such as a wall or a membrane) that is suitable to separate the first environment [A] from the second environment [B] (either in a suitable in vitro system or a suitable in vivo system).

For example, in one preferred aspect of the invention in which the methods of the invention are performed in a suitable cell or cell line (as further described herein), the boundary layer (1) is the cell membrane or cell wall of the cell or cell line that is used in the methods of the invention. In this aspect, the environment [A] is preferably the extracellular environment and the environment [B] is preferably the intracellular environment. Also, in this aspect, the first ligand (3) is preferably present in the extracellular environment and the second ligand (4) is preferably present in the intracellular environment. Also, the first and second binding members (6) and (7) and the second fusion protein are preferably also present in the intracellular environment,

In another preferred aspect of the invention in which the methods of the invention are performed in a suitable vesicle or liposome (as further described herein), the boundary layer (1) is the membrane or wall of the vesicle or liposome. In this aspect, the environment [A] is preferably the environment outside of the vesicle or liposome and the environment [B] is preferably the environment inside the vesicle or liposome. Also, in this aspect, the first ligand (3) is preferably present in the environment outside the vesicle or liposome and the second ligand (4) is preferably present in the environment inside the vesicle or liposome. Also, the first and second binding members (6) and (7) and the second fusion protein are preferably also present in the environment inside the vesicle or liposome.

However, it should be understood that, although the invention in some preferred aspects is performed using cells, liposomes or other suitable vesicles, the invention in its broadest sense is not limited to the use of cells or vesicles but can be performed in any other suitable arrangement in which a boundary layer (1) is used to suitably separate a first environment [A] from a second environment [B]. For example, the boundary layer may also be a part or fragment of a cell wall or cell membrane that is present in a membrane extract, for example a membrane extract that is obtained from whole cells by a technique known per se such as suitable osmotic and/or mechanic techniques known per se.

Thus, the boundary layer (1) can be any suitable layer, wall or membrane, and in particular a biological wall or membrane (such as a cell wall or cell membrane, or a part or fragment thereof) or the wall or membrane of a liposome or other suitable vesicle. In particular, the boundary layer (1) can be a suitable lipid bilayer such as a phospholipid bilayer. When the boundary layer (1) is the wall or membrane of a vesicle or liposome, it can be unilammelar or multilammelar. Also, as further described herein, when the boundary layer (1) is a cell membrane or cell wall, it is preferably the wall or membrane of a cell or cell line that suitably expresses (as defined herein) the translayer protein (2) and in particular suitably expresses a (first) fusion protein as described herein that comprises the translayer protein (2).

As schematically illustrated by the non-limiting FIGS. 1, 2 and 3, the boundary layer (1) contains the translayer protein (2), which spans the boundary layer (1) such that:
the first binding site (8) for the first ligand (3) extends out (as defined herein) into the first environment [A] (i.e. such that the first binding site (8) is accessible for binding by the first ligand (3) when said first ligand is present in the first environment [A]);
   and also such that
the second binding site (9) for the second ligand (4) extends out (as defined herein) into the second environment [B] (i.e. such that the second binding site (9) is accessible for binding by the second ligand (4) when said second ligand is present in the second environment [B]).

In the present description and claims, the term “translayer protein” is used to denote the protein that is used (e.g. that is screened) in the methods and the arrangements of the invention. In the methods and arrangements of the invention, the translayer protein (2) is such (and/or is provided and/or arranged in such a way with respect to the boundary layer) that it spans the boundary layer (1), such that at least one part of the amino acid sequence of the translayer protein (2) extends out (as defined herein) from the boundary layer (1) into the first environment [A] and such that at least one other part of the amino acid sequence of the translayer protein (2) extends out (as defined herein) from the boundary layer (1) into the second environment [B]. In this context, when a part of the amino acid sequence of the translayer protein (2) is said to “extend out” from the boundary layer (1) into an environment (i.e. into the first environment [A] or the second environment [B]), this should generally be understood to mean that said part of the sequence is exposed to said environment and/or is accessible for binding by a ligand, compound or other chemical entity that is present in said environment. Accordingly, in the methods and arrangements of the invention, at least one part of the amino acid sequence of the translayer protein (such as an epitope or binding site) should be accessible for binding by a ligand, compound or other chemical entity that is present in the first environment (and in particular, for binding by the first ligand (3)) and at least one other part of the amino acid sequence of the translayer protein (such as another epitope or binding site) should be accessible for binding by a ligand, compound or other chemical entity that is present in the second environment (and in particular, for binding by the second ligand (4))). In this respect, it should also be noted that the wording “accessible for binding” should generally be taken to mean that a ligand, compound or other chemical entity that is present in the relevant environment can bind to a binding pocket or binding site on or within the translayer protein, even if the actual binding site or binding pocket lies deep(er) within the structure of the translayer protein (even such that the actual binding site or binding pocket is located within a part of the translayer protein that itself does not physically extend out beyond the boundary layer). Reference is for example made to the publication by Chevillard (cited herein) which shows that the binding sites on GPCRs for fragments that are used in FBDD screening techniques may lie deep within the GPCR structure (see for example FIG. 2 on page 1120) and not be on the surface of the GPCR, but nevertheless are accessible for fragment binding. Reference is also made to the teachings on GPCR structure, GPCR signaling mechanisms and GPCR ligand binding sites from some of the other scientific references cited herein,

Also, in the present description and claims, when any binding domain, binding unit, epitope, binding site, ligand, protein or other compound or chemical or other structural entity (such as a protein complex) is said to be “present in” an environment (i.e. in the first environment [A] or the second environment [B]), this should generally be understood to mean that said binding domain, binding unit, epitope, binding site, ligand, protein or other compound or chemical or structural entity is exposed to said environment and/or is accessible for binding by another domain, ligand, protein or compound that is present in said environment. Thus, for example, a compound or ligand that is present in an environment may either be “free-floating” in said environment (i.e. not be bound or anchored to any other protein or structure) or may be anchored to the boundary layer or fused to another protein (which other protein may be anchored to the boundary layer). Similarly, a binding domain or binding unit that is present in an environment may be part of a larger protein or structure (such as a fusion protein), which larger structure may be free-floating in said environment or be anchored to the boundary layer or to another structure, as long as the binding domain or binding unit is accessible for binding by another domain, ligand, protein or compound that is present in said environment. Also, an epitope or binding site that is present in an environment may be part of a larger protein or structure, which larger protein or structure may again be free-floating in said environment or be anchored to the boundary layer or to another structure, as long as the epitope or binding site is accessible for binding by another domain, ligand, protein or compound that is present in said environment.

The part or parts of the translayer protein (2) that extend out into the first environment [A] can be any loop, epitope (linear or conformational), binding site or other part(s) of the amino acid sequence of the translayer protein, and similarly the part or parts of the translayer protein that extend out into the second environment [B] can also be any loop, epitope (linear or conformational), binding site or other part(s) of the amino acid sequence of the translayer protein (but will be different from the part(s) that extend out into the first environment).

In a preferred aspect of the invention, the translayer protein (2) comprises at least two different/distinct ligand binding sites, of which at least a first binding site extends out (as defined herein) into the first environment [A] (in particular such that it is accessible for binding by the first ligand (3)) and of which at least a second binding site extends out (as defined herein) into the second environment [B] (in particular such that it is accessible for binding by the second ligand (4)).

Generally, the translayer protein (2) will usually be attached to and/or anchored in the boundary layer (1), for example in a manner that is known per se for (trans)membrane proteins that in their native environment are anchored in a cell wall or cell membrane. As further described herein, this can for example be achieved by suitably expressing (as defined herein) a nucleotide sequence or nucleic acid that expresses the first fusion protein in a suitable host cell such that the translayer protein (2) becomes suitably anchored in the wall or membrane of said cell. When the method of the invention is performed using a liposome or vesicle, this can be achieved by suitably forming said liposome or vesicle in the presence of the first fusion protein such that the translayer protein (2) becomes suitably anchored into the wall or membrane of the liposome or vesicle.

The translayer protein (2) can comprise one or more domains (and in particular one or more transmembrane domains) and will usually, and preferably, be a transmembrane protein, such as a (transmembrane) receptor.

When the translayer protein (2) is a transmembrane protein, it can be bitopic membrane protein (i.e. a transmembrane protein with a single pass through the membrane) or a polytopic membrane protein (i.e. a transmembrane protein with two or more passes through the membrane). As such, the translayer protein (2) can be any known or newly discovered transmembrane protein (or a synthetic or recombinant analog thereof), with known or unknown biological functions, and with known or unknown ligands (for example, the translayer protein (2) can be a so-called “orphan” GPCR).

The translayer protein (2) can be an alpha-helical protein or a beta-barrel protein, and can be a Type I, Type II, Type III or Type IV transmembrane protein, depending on the position of the N-terminus and the C-terminus of the protein relative to the boundary layer. Preferably, and although the invention in its broadest sense is not limited thereto, the translayer protein is a protein that, in its native cellular environment, has its amino-terminus outside of the cell and its carboxy terminus inside the cell.

Also, when the methods of the invention are performed in cells, the arrangement of the N-terminus and the C-terminus of the protein relative to the wall or membrane of the cell used are preferably the same as the arrangement of said termini in the native cellular environment of the protein.

When the methods of the invention are to be performed in liposomes or vesicles, it may be that the liposomes or vesicles may be a mixture of liposomes/vesicles in which the protein is arranged in a way that is essentially the same as the way that the protein is arranged with respect to the cell wall or cell membrane in its native environment (i.e. with the N-terminus and the extracellular loop(s) extending to the outside of the vesicle and the C-terminus and the intracellular loop(s) extending to the inside of the vesicle) and vesicles/liposomes in which the protein is arranged the other way around. Usually, this will not affect the performance of the system or set-up described herein.

As further described herein, generally and preferably, the translayer protein (2) will be a protein that exists (i.e. can take on) two or more conformations (such as a basal state/conformation, an active state/conformation and/or an inactive state/conformation, and/or a ligand-bound or ligand-free conformation) and/or a protein that can undergo a conformational change (and in particular, a functional conformational change). In particular, the translayer protein (2) can be a protein that can take on at least one functional conformation and at least one non-functional conformation (such as a basal conformation) and/or that can undergo a conformational change from a non-functional conformation into a functional conformation; and more in particular a protein that can take on an active (or more active) conformation and an inactive (or less active) conformation and/or that can undergo a conformational change from an inactive (or less active) conformation into an active (or more active) conformation. The translayer protein (2) can also be a protein that can take on at least one ligand-bound (and in particular agonist-bound) conformation and at least one ligand-free conformation. More in particular, the translayer protein (2) can be a protein that can take on at least one ligand-bound (and in particular agonist-bound) conformation that is an active or functional conformation.

As described herein, a particular class of functional conformations of (transmembrane) proteins (such as certain GPCRs) is referred to/defined as “druggable conformation”. Thus, in one specific aspect, the translayer protein (2) can be a protein that can take on at least one such druggable conformation (which will often be an active conformation, although the invention is not limited to use with druggable conformations that are active conformations) and at least one conformation that is not a druggable conformation (which will often be an inactive conformation) and/or a translayer protein that can undergo a conformational change from a non-druggable conformation to a druggable conformation.

In particular, the translayer protein (2) can be a protein that undergoes a conformational change upon binding of a ligand (and in particular an agonist) to the protein. This conformational change upon binding of the ligand can for example be a conformational change from an active conformation into an inactive conformation or from a functional conformation to a non-functional conformation, but is preferably a change from a non-functional conformation to functional conformation and/or an inactive conformation to an active conformation. In a particular aspect, it is a change from a non-druggable conformation into a druggable conformation.

For example, when the translayer protein (2) is a receptor such as a cell surface receptor (or a synthetic analog thereof), it can be a protein that undergoes a conformational change when a natural or synthetic (extracellular) ligand of the receptor binds to the receptor.

When the translayer protein (2) is a GPCR, the conformational change may in a preferred but non-limiting aspect be a change from a conformation that is essentially not capable of binding G-protein into a conformation that binds G-protein (or is capable of being bound by G-protein).

As mentioned herein, a ligand that is capable of eliciting a conformational change in the translayer protein (2) from a non-functional state into a functional state (for example from an inactive state such as a basal state into an active state) is also referred to herein as an “agonist” of the translayer protein. When the translayer protein (2) is a GPCR, an “agonist” may in particular be capable of eliciting a conformational change from a conformation that is essentially not capable of binding G-protein into a conformation that binds G-protein.

In one preferred aspect of the invention, the translayer protein (2) is a protein that undergoes (or is capable of undergoing) a conformational change (as described herein) when the first ligand (3) binds to it and conversely the first ligand (3) is such that it can invoke a conformational change in the translayer protein (2) when it binds to it (and/or the invention is used to identify such first ligands. Again, in one more preferred aspect, said conformational change is a change from an inactive or less active state to a functional or (more) active state and the first ligand (3) used is such that, when it binds to the translayer protein (2), it can invoke a conformational change in the translayer protein from an inactive or less active state into a functional or (more) active state. Also, when the translayer protein (2) is a GPCR, the conformational change upon binding of the ligand may in a preferred but non-limiting aspect be a change from a conformation that is essentially not capable of binding G-protein into a conformation that binds G-protein.

As also further described herein, the translayer protein can be a protein that can form a complex with a first and a second ligand. In particular, the translayer protein can be a protein that, in its native environment, can form a complex with an intracellular ligand and an extracellular ligand. For example, from the references cited herein, it is known that most GPCRs form a complex with an extracellular ligand and the G-protein (which is the most common native intracellular ligand for a GPCR), and that such a complex is stabilized by the G-protein binding to the intracellular conformational epitope of the GPCR. Similarly, in the invention, the second ligand is preferably such that it stabilizes (the formation of) a complex of the translayer protein, the first ligand and the second ligand. For example, for this purpose, and as further described herein, when the translayer protein (2) is a GPCR, the second ligand may be the G-protein that is associated with the GPCR in its native environment (i.e. with signal transduction by the GPCR), another naturally occurring G-protein that is capable of binding to the GPCR and stabilizing the formation of the aforementioned complex, or a synthetic or semi-synthetic analog or derivative of a GPCR that is capable binding to the GPCR and stabilizing the formation of the aforementioned complex. As also mentioned herein, the second ligand may be a ConfoBody, i.e. an immunoglobulin single variable domain (such as a VHH or Nanobody) that has been designed/raised to stabilize the formation of the complex of the ConfoBody, the translayer protein and the first ligand.

In one preferred but non-limiting aspect of the invention, the translayer protein (2) will be a “seven-pass-transmembrane protein”, and in particular a 7TM that is a receptor (such as a cell surface receptor). In a particularly preferred aspect, the translayer protein (2) can be a 7TM that signals through G-protein. Such 7TMs are also known in the art as GPCRs [As mentioned, the terms “GPCR” and “7TM” are used interchangeably herein to include all transmembrane proteins with 7-transmembrane domains, irrespective of their intracellular signaling cascade or signal transduction mechanism, although it should be understood that throughout the description and claims, 7 TMs that signal through G-proteins are a preferred aspect of the invention].

The translayer protein (2) can be a naturally occurring protein or receptor or a synthetic or semi-synthetic analog of a naturally occurring protein or receptor (again obtained through protein chemistry or recombinant DNA technology as generally described herein). Such a synthetic analog can for example be an analog of a naturally occurring transmembrane protein in which, compared to the sequence of the naturally occurring protein, one or more one or more amino acid residues or stretches of amino acid residues (including one or more loops or parts thereof and/or one or more domains and/or parts thereof) have been inserted, deleted and/or replaced by other amino acid residues or stretches of amino acid residues (for example, by essentially corresponding stretches or loops of amino acids from other (preferably structurally related) membrane proteins (in other words, that contain one or more “amino acid differences”—as defined herein—compared to the native sequence). Often, the native sequence of the naturally occurring protein used will be obtained from the species to be treated with a compound of the invention or from an animal (preferably a mammal) that is to be used for the purposes of an animal model for testing a compound of the invention.

As will be clear to the skilled person, such synthetic analogs can be obtained using standard techniques of protein chemistry and/or standard techniques of protein chemistry and/or recombinant DNA technology known per se. For example, when the invention is performed in cells as described herein, the synthetic analog can be obtained by suitably expressing in said cell a DNA sequence (or other suitable nucleotide sequence) that encodes said synthetic analog.

Also, as is well known in the art, 7TMs and other transmembrane proteins usually comprise one or more intracellular loops and one or more extracellular loops. Similarly, the translayer protein used in the arrangements of the invention may comprise one or more loops that extend out (as defined herein) in to the first environment and one or more loops that extend out (as defined herein) into the second environment. For example, when the methods of the invention are performed in cells, the translayer protein used in the arrangements of the invention may comprise one or more loops that extend out into the intracellular environment and one or more loops that extend out into the extracellular environment. Similarly, when the methods of the invention are performed in vesicles or liposomes, the translayer protein used in the arrangements of the invention may comprise one or more loops that extend out into the environment inside of the liposome or vesicle and one or more loops that extend out into the environment outside of the liposome or vesicle. In each case, the loops that extend out into the first environment are most preferably such (and arranged such) that they can form a functional ligand binding site (and in particular a functional binding site for the first ligand) and/or such that they can do so when the translayer protein takes on a suitable conformation; and the loops that extend out into the second environment are most preferably such (and arranged such) that they can form a functional ligand binding site (and in particular a functional binding site for second ligand) and/or such that they can do so when the translayer protein takes on a suitable conformation (for example, upon binding of a first ligand to the translayer protein).

In one specific but non-limiting aspect, the loops of the translayer protein that extend out into one environment will essentially correspond to extracellular loops of a translayer protein and the loops of the translayer protein that extend out into the other environment will essentially correspond to intracellular loops (again, in each case preferably such that the extracellular loops will form a functional ligand binding site and such that the intracellular loops will form another functional ligand binding site). Preferably, the loops of the translayer protein that extend out into the first environment [A] will essentially correspond to the extracellular loops of a translayer protein and the loops of the translayer protein that extend out into the second environment [B] will essentially correspond to the intracellular loops of a translayer protein, in particular when the second environment [B] is the environment inside of a cell or liposome (and again, preferably such that the extracellular loops will form a functional ligand binding site that extends out into the first environment and such that the intracellular loops will form a different functional ligand binding site that extends out into the second environment).

For example, when the translayer protein is a transmembrane protein (such as a 7TM), the translayer protein may comprise one or more extracellular loops of a transmembrane protein (and in particular one or more extracellular loops of a 7TM) and one or more intracellular loops of a transmembrane protein (and in particular one or more intracellular loops of a 7TM), more in particular such that said extracellular loops form or can form a functional ligand binding site and such that said intracellular loops form or can form a different functional ligand binding site. Again, the ligand binding site formed by said extracellular loops will preferably extend out (as defined herein) into one environment and the ligand binding site formed by said intracellular loops will preferably extend out (as defined herein) into the other environment. In particular, when the methods of the invention are performed in cells or liposomes, said extracellular loops will extend out into the environment outside of the cell or liposome and said intracellular loops will extend out into the environment inside of the cell or liposome. Also, preferably, the intracellular loops are such (and arranged such) that they form or can form a functional ligand binding site for the second ligand (or put in other words: in the invention, the ligand binding site for the second ligand is preferably made up by and/or comprises one or more intracellular loops of a transmembrane protein. It may also be the case that the ligand binding site for the first ligand is made up by and/or comprises one or more extracellular loops, but as further described herein, it is also possible that the actual binding/docking site for the first ligand lies deeper within the structure of translayer protein).

For example, when the translayer protein is a 7TM, the translayer protein may comprise three intracellular loops (i.e. three intracellular loops from a 7TM) and three extracellular loops (i.e. three extracellular loops from a 7TM), in which three intracellular loops form or can form a functional ligand binding site and in which the three extracellular loops form or can form a different functional ligand binding site. Again, preferably, the functional ligand binding site that is formed by the three intracellular loops extends out into one environment (and preferably the second environment [B]) and the functional ligand binding site that is formed by the three extracellular loops extends out into the other environment (and preferably the second environment [A]). Also, the three intracellular loops preferably form a functional binding site for the second ligand (and the three extracellular loops may form a functional binding site for the first ligand or said binding site may lie deeper within the structure of the 7TM). Most preferably, the three intracellular loops will form a binding site for the second ligand that extends out into the second environment [B] (i.e. the environment inside of the cell or liposome when the methods of the invention are performed in cells or liposomes, respectively) and the three extracellular loops will extend out into the first environment [A] (and may form a functional binding site for the first ligand or said binding site may lie deeper within the structure of the 7TM).

In one aspect of the invention, the intracellular and extracellular loops of the translayer protein are derived or essentially derived from the same transmembrane protein (i.e. are the same or essentially as those that are present in the native transmembrane protein). In this aspect of the invention, the translayer protein may have the same or essentially the same amino acid sequence as the native transmembrane protein that is to be used as a target in the screening or assay methods of the invention.

In another aspect of the invention, the intracellular and extracellular loops of the translayer protein may be derived from different transmembrane proteins. In particular, in this aspect of the invention, the intracellular and extracellular loops may be derived from different but related transmembrane proteins, for example from two different but related 7TMs such as two GPCRs. In particular, in this aspect of the invention, the intracellular loops may be derived from a first 7TM or GPCR, and the extracellular loops may be derived from a second 7TM or GPCR different from the first. The transmembrane domains of such a chimeric protein may be derived from the first or the second 7TM or GPCR, and are preferably essentially all derived from the same GPCR, and are more preferably derived from the same GPCR as the extracellular loops (but may contain some amino acid residues from the GPCR from which the intracellular loops have been derived, depending on the positions chosen for recombinantly deleting the native intracellular loops and inserting the replacement intracellular loops).

In this aspect of the invention, the resulting chimeric translayer protein should most preferably still be such that it can be suitably used in the methods and arrangements of the invention. Also, again, in the case of a 7TM, the translayer protein will comprise three intracellular loops and three extracellular loops, with the three intracellular loops forming a functional ligand binding site for the second ligand (which second ligand will then be selected such that it can bind to the ligand binding site (9) that is formed by said intracellular loops). Again, the binding site that is formed by the three intracellular loops will preferably extend out into the second environment [B] (i.e. the environment inside of the cell or liposome when the methods of the invention are performed in cells or liposomes, respectively) and the three extracellular loops will preferably extend out into the first environment [A] (and may form a functional binding site for the first ligand or said binding site may lie deeper within the structure of the 7TM).

Thus, in a further aspect, the invention relates to an arrangement as further described herein, in which the translayer protein is a 7TM that comprises 7 transmembrane domains, 3 intracellular loops and 3 extracellular loops (which are linked to each other and in an order as is known per se for 7TMs, i.e. [N-terminal sequence]-[TM1]-[IC1]-[TM2]-[EC1]-[TM3]-[IC2]-[TM4]-[EC2]-[TM5]-[IC3]-[TM6]-[EC3]-[TM7]-C-terminal sequence), in which the intracellular loops are derived from a first 7TM and the extracellular loops are derived from a second 7TM different from the first 7TM, in which the intracellular loops form a functional ligand binding site. Preferably, the TM domains from said translayer protein are essentially derived from the same 7TM as the extracellular loops.

Also, said intracellular loops and the 7TM as a whole are such that they form a functional ligand binding site, and in particular a functional ligand binding site to which a (suitable) second ligand (as defined herein) can bind. Said ligand binding site again preferably extends out into the second environment [B].

In one specific aspect, such a chimeric translayer protein comprises intracellular loops that have been derived from the beta-2-adrenegic receptor. In another specific aspect, such a chimeric translayer protein comprises intracellular loops that have been derived from the Mu-opioid receptor. For some non-limiting examples of such chimeric receptors, reference is also made to the co-pending PCT application by assignee entitled “Chimeric proteins and methods to screen for compounds and ligands binding to GPCRs” which has the same international filing date and invokes the same priority applications as the present application.

The invention in particular relates to an arrangement that comprises such a chimeric 7TM and a second ligand that can bind to the ligand binding site that is formed by said intracellular loops.

For the remainder, provided that the second ligand is suitably chosen such that it can bind to the ligand binding site (9) on the chimeric translayer protein so as to provide an operable arrangement of the invention (and provided that the chimeric translayer protein itself is operable in the arrangement of the invention), such arrangements of the invention in which a chimeric translayer protein is used can be essentially as further described herein.

Also, such chimeric translayer proteins, nucleotide sequences and nucleic acids that encode the same, and cells, cell lines or host organisms that contain such nucleotide sequences or nucleic acids and/or that can express such chimeric translayer proteins form further aspects of the invention, as do further uses of such chimeric translayer proteins, nucleotide sequences, nucleic acids, cells, cell lines and host organisms.

Another aspect of the invention is a composition or kit-of-parts that comprises at least said chimeric translayer protein and a ligand that can bind to the intracellular loops that are present in said GPCRs. Said ligand is preferably a protein and more preferably a protein that comprises or essentially consists of an immunoglobulin single variable domain (such as a VHH domain) and may in particular be a ConfoBody (as described herein).

As mentioned, the chimeric translayer protein is preferably a 7TM/GPCR. Also, in one specific aspect, said chimeric translayer protein comprises intracellular loops that have been derived from the beta-2-adrenegic receptor. In another specific aspect, said chimeric translayer protein comprises intracellular loops that have been derived from the Mu-opioid receptor.

As further described herein, and as schematically shown in FIGS. 1 to 3, in the arrangements of the invention, the translayer protein (2) is usually, and preferably, fused or linked, either directly or via a suitable spacer or linker (10), to the first member (6) of the binding pair (6/7) so as to form a first fusion protein. Also, the second binding member (7) of the binding pair (6/7) will usually, and preferably, be part of a second fusion protein that is different from the first fusion protein, which second fusion protein is also as further described herein. Said first fusion protein, said second fusion protein (in its various formats as described herein), nucleotide sequences and/or nucleic acids that encode the first or second fusion protein, and cells, cell lines or other host cells or host organisms that express (and in particularly suitably express, as described herein) or are capable of (suitably) expressing the first and/or the second fusion protein (and preferably both), as well as the various uses of the same as further described herein, form further aspects of the invention.

The binding pair (6/7) that is used in the arrangements of the invention will generally comprise at least two separate binding members (6) and (7), which are also referred to herein as the “first binding member” and the “second binding member”, respectively. The binding pair (6/7) and each member (6) and (7) thereof should be such that the binding pair (6/7) is capable of generating a detectable signal when the members (6) and (7) come into contact or in close proximity to each other. Such a detectable signal can for example be a luminescent signal, fluorescence signal or chemiluminescense signal, be based on a reporter gene, or on DNA ligation. Some specific but non-limiting examples of techniques (including binding pairs and their associated detectable signals) are techniques based on protein complementation such as the NanoBit™ system, the NanoLuc™ system, the hGLuc system (Remy and Michnick, Nature Methods, 2006, 977), BiFC (bimolecular fluorescence complementation) and DHFR-PCA (dihydrofolate reductase protein-fragment complementation assay); techniques based on direct interaction such as BRET (bioluminescence resonance energy transfer), FRET (fluorescence/Foerster resonance energy transfer) and BioID (proximity-dependent biotin identification); systems based on reporter genes (such as KISS/kinase substrate sensor) or proximity ligation assays (Weilbrecht et al., Expert Review of Proteomics, 7:3, 401-409). Techniques based on protein complementation and a a luminescent signal, fluorescence signal or chemiluminescense signal (such as NanoLuc™ or NanoBit™) will usually be preferred.

In one specifically preferred aspect, when the methods of the invention are performed in a suitable cell, the first member (6) and the second member (7) of the binding pair (6/7) are preferably both a polypeptide, protein, amino acid sequence or other chemical entity that can be obtained by suitably expressing, preferably in the cell that is used in the method of the invention, a nucleic acid or nucleotide sequence that encodes the same.

The first and second binding members can also be part of a suitable reporter assay, can be an enzyme-and-substrate combination, or any other pair of domain or units that can generate a detectable signal when they come into contact with, or close proximity to, each other, such as binding pairs that are commonly used in experimental study of protein-protein interactions. As mentioned, to reduce the level of baseline/background signal, it is preferred that the two members of the binding pair by themselves do not have a substantial binding affinity for each other.

Some preferred but non-limiting examples of suitable binding pairs are pGFP and the NanoBiT® system from Promega. The latter is especially preferred because the Large BiT and the small BiT that make up the NanoBiT® system by themselves have low affinity for each other.

The first binding member (6) can be fused in any suitable manner to the translayer protein (2), as long as the resulting first fusion protein is such that it allows the first member (6) to come into contact with (or otherwise suitably in close proximity to) the second member (7) of the binding pair (6/7) when the second fusion protein formed by the second ligand (4) and the second member (7) binds to the translayer protein (2) via the second binding site (9). Also, preferably, first binding member (6) is fused or linked to the translayer protein (2) in a way that essentially does not affect, under the conditions used to perform the methods of the invention, the conformations and/or conformational changes that the translayer protein (2) can undergo.

Thus, generally, although it is not excluded in the invention that the first binding member (6) is fused or linked directly to the translayer protein (2), it is generally preferred that the first binding member (6) is fused or linked to the translayer protein (2) via a suitable linker (10). The use of a flexible linker, for example with a total of between 5 and 50 amino acids, preferably between 10 and 30 amino acids, such as about 15 to 20 amino acids, is usually preferred. Suitable linkers will be clear to the skilled person and include GlySer linkers (for example a 15GS linker).

In the invention, the first and second binding members of the binding pair (6/7) will be present in (as defined herein) the same environment relative to the boundary layer (1), such that they can come into contact or close proximity to each other (in the manner as further described herein) and upon doing so can generate a detectable signal. In particular, as schematically shown in FIGS. 1, 2 and 3, the first and second binding members of the binding pair (6/7) will be present in (as defined herein) the same environment as the second binding site (9) on the translayer protein (2) (again, relative to the boundary layer (1)), so as to allow first and second binding members of the binding pair (6/7) to come into contact when the second fusion protein binds to said binding site, i.e. either directly (as shown in FIG. 1) or indirectly (as shown in FIGS. 2 and 3). For this, the first binding member (6) will generally be attached, directly or via linker (10), to an amino acid residue/position in/on the translayer protein (2) that is exposed to the same environment as the second binding site (9). As further described herein, said environment (indicated as environment [B] in FIGS. 1 to 3) can for example be the intracellular environment (when the method of the invention is performed in cells) or the environment inside a vesicle or liposome.

In a preferred aspect of the invention, the first binding member (6) will be fused, directly or via the linker (10), to one end of the primary amino sequence of the translayer protein (2). This may be the N-terminus or the C-terminus of the translayer protein (2), again as long as in the final arrangement of the invention the first binding member (6) is on the same side of the boundary layer (1) as the second binding site (9). Accordingly, in the aspect of the invention that is performed in cells as further described herein, and where the second binding site (9) is exposed to the intracellular environment, the first member (6) may be fused to the end of the primary amino acid sequence that terminates in the intracellular environment (which, in the case of 7TMs, will usually be the C-terminal end).

The first fusion protein may be provided and produced using suitable techniques of protein chemistry and/or recombinant DNA technology known per se. Such techniques will be clear to the skilled person based on the further disclosure herein as well as the standard handbooks and other scientific references referred to herein. When the method of the invention is performed in cells (as further described herein), the first fusion protein is preferably provided by suitably expressing, in said cell, a nucleotide sequence and/or nucleic acid that encodes the first fusion protein. This can again be performed using suitable techniques of recombinant DNA technology known per se, and cells that suitably express or (are capable of suitably expressing) the first fusion protein form a further aspect of the invention.

As further described herein, in the arrangements of the invention, the second member (7) of the binding pair (6/7) will usually and preferably also form part of a fusion protein, which fusion protein will generally comprise said second binding unit which is fused or linked, either directly or via a suitable spacer or linker (11), to another ligand, protein, binding domain or binding unit, which ligand, protein, binding domain or binding unit is such that it can bind directly (as defined herein) or indirectly (as defined herein) to the translayer protein (2). For this purpose, as further described herein, said ligand, protein, binding domain or binding unit may for example be the second ligand (resulting in an arrangement of the invention of the type that is schematically shown in FIG. 1, with (4) being the second ligand), be a binding domain or binding unit that can bind to the second ligand (resulting in an arrangement of the invention of the type that is schematically shown in FIG. 2, with (4) being the second ligand and (5) being the binding domain or binding unit binding to the second ligand), or be a binding domain or binding unit that can bind to a protein complex that can bind to the translayer protein (as schematically shown in FIG. 3, with (4) being the second ligand, (12) being said protein complex comprising the second ligand, and (5) being the binding domain or binding unit that binds to the protein complex).

In the second fusion protein, the second binding member (7) is most preferably linked to said other ligand, protein, binding domain or binding unit in a suitable manner that allows the second binding member (7) to come into contact with (or otherwise suitably in close proximity to) the first member (6) of the binding pair (6/7) when the second fusion protein binds directly or indirectly to the second binding site (9) on the translayer protein (2). For this, the second binding member (7) may be fused or linked directly to said other ligand, protein, binding domain or binding unit, but preferably they are linked via a suitable linker (11), which is preferably a flexible linker, for example with a total of between 5 and 50 amino acids, preferably between 10 and 30 amino acids, such as about 15 to 20 amino acids, is usually preferred. Suitable linkers will be clear to the skilled person and include GlySer linkers (for example a 15GS linker).

As mentioned herein, the second ligand can be any ligand, protein, binding domain or binding unit is capable of binding to the translayer protein, i.e. via the binding site (9) (when the second ligand is part of the second fusion protein, it should most preferably also be such that it can be suitably included in the second fusion protein).

Generally, in the invention (and irrespective of whether said binding site is bound directly or indirectly by the second fusion protein used in the arrangements of the invention), the binding site (9) can be a conformational epitope on the translayer protein (2). More in particular, said binding site (9) can be a conformational epitope on the translayer protein (2) that changes it “shape” (i.e. the spatial arrangement of the domains, loops and/or amino acid residues that form the epitope) when the translayer protein (2) undergoes a conformational change, for example a conformational change from an inactive or less active state into an active, more active and/or functional state and/or a conformational change that occurs when a first ligand binds to the translayer protein.

Preferably, the binding site (9) and the second ligand are such that the affinity for the interaction between the binding site (9) and the second ligand (4) changes when the binding site (9) changes it shape because the translayer protein (2) undergoes a conformational shape. In particular, the binding site (9) and the second ligand may be such that the affinity for the interaction between the binding site (9) and the second ligand (4) increases when the translayer protein (2) undergoes a conformational change from an inactive or less active state into an active, more active, functional and/or druggable state and/or undergoes a conformational change that occurs when a first ligand (3) (and in particular a first ligand (3) that acts as an agonist in respect of the translayer protein) binds to the translayer protein (2).

In particular, the second ligand (4) and its interaction with the binding site (9) may be such that the second ligand (4) binds with higher affinity to the binding site (9) when the translayer protein (2) is an active, more active and/or functional state and/or such that the second ligand (4) binds with higher affinity to the binding site (9) when a first ligand (3) (and in particular a first ligand (3) that acts as an agonist with respect to the translayer protein (2)) binds to the translayer protein (2). For example, the second ligand (4) and its interaction with the binding site (9) can be such that the affinity of the second ligand (4) for the translayer protein (2) increases 10 fold, such as 100 fold or more, when the translayer protein (2) undergoes such a conformational change, for example from an affinity in the micromolar range (i.e. more than 1000 nM) when the translayer protein is in an inactive, less active or ligand-free conformation to an affinity in the nanomolar range (i.e. less than 1000 nM, such as less than 100 nM) when the translayer protein (2) is in a functional, active or more active and/or ligand-bound conformation. For example, in the case of a GPCR, it is known that the affinity for the interaction between the G-protein and the G-protein binding site increases when a ligand (and in particular an agonist) binds to the extracellular binding site of the GPCR. Also, WO2012/007593, WO2012/007594, WO2012/75643, WO 2014/118297, WO2014/122183 and WO 2014/118297 describe VHH domains (ConfoBodies) that have higher affinity for a GPCR when the GPCR is in a functional, active or more active and/or ligand-bound conformation compared to when the translayer protein is in an inactive, less active or ligand-free conformation (e.g. in the nanomolar range for a functional, active or ligand-bound conformation vs in the micromolar range for an inactive or ligand-free conformation).

It is also possible that the second ligand itself undergoes a conformational change when it binds to the translayer protein (2). In the embodiments where the second fusion protein binds indirectly to the translayer protein (2), this may also mean that the binding domain or binding unit (5) in the second fusion protein that binds to the second ligand (4) may be such that it has higher affinity for the conformation that the second ligand (4) adopts when the second ligand (4) is bound to the translayer protein (2) compared to the conformation that the second ligand (4) adopts when it is not bound to the translayer protein (2). For example, it is known that the G-protein undergoes a conformational change when it binds to a GPCR, and it may be that the VHH domain that is present in the second fusion protein has higher affinity for the GPCR-bound conformation of the G-protein compared to the unbound conformation of the GPCR.

In one preferred aspect, the binding site (9) is a binding site on the translayer protein (2) that, when the translayer protein is in its natural environment, serves as a binding site for a natural ligand of the translayer protein. More in particular, the binding site (9) can be a binding site on the translayer protein (2) that, when the translayer protein is in its natural environment, serves as an intracellular binding site for a natural intracellular ligand of the translayer protein. For example, when the translayer protein (2) is a receptor, the binding site (9) can be a binding site on the translayer protein (2) that, when the translayer protein is in its natural environment, serves as an intracellular binding site for one or more intracellular ligands of the translayer protein (2) that are involved in signal transduction.

In a specific aspect, when the translayer protein (2) is a GPCR, the binding site (9) can be the binding site for the G-protein (and/or for a G-protein complex). As further described herein, in such a case, the second ligand can be a natural, synthetic or recombinant protein or other ligand that can bind to the G-protein binding site on the GPCR.

The second ligand (4) will usually be a protein or a proteinaceous ligand. In the aspects of the invention that are performed in a suitable cell or cell line, the second ligand (4) may be a protein that is native to the cell or cell line used or may be a suitable (recombinant) protein that is expressed in the cell or cell line used. For example, when the second ligand (4) is not part of the second fusion protein, it can be a ligand of the translayer protein (2) that naturally occurs in said cell or cell line (for example, when the translayer protein (2) is a GPCR, the second ligand (4) may be a G-protein that is natively expressed by the cell or cell line used). Alternatively, the second ligand may be a protein that is recombinantly expressed in the cell or cell line used, for example when said cell or cell line does not natively express a suitable ligand for the translayer protein (2) or when it is desired to use a ligand that is different from the ligand(s) that natively are expressed by said cell or cell line (for example, when it is desired to use an analog, derivative or ortholog of the natively expressed ligand, in which case the native expression of the natively expressed ligand may also be temporarily or constitutively suppressed or knocked-out in the cell or cell line used). When the second ligand (4) forms part of the second fusion protein, the second ligand will usually be expressed recombinantly as part of the second fusion protein.

As further described herein, the second ligand (4) can either be part of the second fusion protein or it can be separate from the second fusion protein. In either case (i.e. irrespective of whether the second ligand is part of the second fusion protein or not), the second ligand is preferably such that it is capable of binding to a conformational epitope on the translayer protein (or such that it is part of a protein complex that binds directly to the translayer protein or that is capable of binding directly to the translayer protein). More preferably, the second ligand (and/or the protein complex that comprises the second ligand) is preferably such that it specifically binds to one or more functional, active and/or druggable conformations of the translayer protein, such that it induces the formation of and/or stabilizes one or more functional, active and/or druggable conformations of the translayer protein (and/or shifts the conformational equilibrium of the translayer protein towards one or more such conformations); and/or such that it induces the formation of and/or stabilizes a complex of the translayer protein, the first ligand and the second ligand.

When the second ligand is part of the second fusion protein, it can be any ligand, binding domain, binding unit, peptide, protein or other chemical entity that can bind directly to the translayer protein and that can suitably be included in the second fusion protein. Preferably, as further described herein, when it is part of the second fusion protein, the second ligand will be a suitable binding domain or binding unit, and in particular an immunoglobulin single variable domain.

When the second ligand is separate from the second fusion protein, it can be any ligand or protein that can bind directly to the translayer protein and/or that can form part of a protein complex that can bind to the translayer protein. For example, as further described herein, such a second ligand may be a naturally occurring ligand of the translayer protein, a semi-synthetic or synthetic analog or derivative of such a naturally occurring ligand or an ortholog of such a naturally occurring ligand. Also, when the second ligand is not part of the second fusion protein, the second fusion protein will comprise a binding domain or binding unit that can bind indirectly (as defined herein) to the translayer protein, i.e. a binding domain or binding unit that can bind to the second ligand and/or to a protein complex that comprises the second ligand. Again, as also further described herein, such a binding domain or binding unit may in particular be an immunoglobulin single variable domain, such as a camelid-derived ISVD. As also mentioned herein, such a binding domain or binding unit can comprise two or more (such as two or three) ISVD's (suitably fused or linked, optionally via a suitable linker or spacer) which can be the same or different and which—when the same—will generally bind to the same binding site or epitope on the second ligand or which—when different—can bind to the same or different epitope or binding site on the second ligand (and also, when the second ligand is a protein complex such as a G-protein complex, can bind to the same or to different subunits of said protein complex).

It will also be clear to the skilled person that, when the second ligand does not form part of the second fusion protein, that the binding domain or binding unit that is present in the second fusion protein and that can bind to the second ligand should essentially not interfere with the binding of the second ligand to the translayer protein. For example, it is preferably such that it binds to a binding site or epitope on said second ligand that is distinct from the binding site on the second protein that binds to the translayer protein (and preferably also sufficiently removed from the binding site on the second protein that binds to the translayer protein so as to avoid any major steric hindrance).

When the second ligand (4) is a naturally occurring ligand of the translayer protein (2), it may for example be a ligand that is involved in the signaling pathway or signaling transduction in which the translayer protein (2) is involved. For example, when the second ligand (4) is a receptor, the second ligand (4) may be a naturally occurring ligand of the receptor, and in particular a naturally occurring intracellular ligand of the receptor, for example an intracellular ligand that binds to an intracellular binding site on the receptor when an extracellular ligand binds to an extracellular binding site on the receptor or, when the receptor has some degree of constitutive activity, that binds to an intracellular binding site of the receptor as part of the pathway that provides said constitutive activity. Suitable examples of such a natural ligand will be clear to the skilled person based on the disclosure herein and will generally depend on the translayer protein (2) used. For example, when the translayer protein (2) is a 7TM or GPCR, the second ligand (4) may be a G-protein (preferred), including but not limited to a naturally occurring G-protein (such as a G-protein that naturally occurs in the cell or cell line used) or a synthetic or semi-synthetic analog or derivative of a naturally occurring G-protein (including chimeric G-proteins), all as further described herein.

As further described herein, and in particular in aspects and embodiments of the invention that are performed using a cell or cell line, the second ligand (4) may also be part of a complex that comprises the second ligand (4) and optionally one or more further proteins. For example, when the translayer protein is a GPCR and the second ligand is a G-protein or an analog or derivative of a G-protein, the second ligand may be part of a complex formed by said G-protein and optionally one or more further proteins. One preferred but non-limiting example of such a complex is the G-protein trimer comprising a G-alpha subunit, a G-beta subunit and a G-gamma subunit. Said complex may also comprise the translayer protein itself (e.g. the GPCR and the G-protein or the GPCR and the G-protein trimer). It will be clear to the skilled person that, when the second ligand forms part of such a complex, it is generally preferred that the second ligand does not form part of the second fusion protein. Instead, the second fusion protein will comprise a binding domain or binding unit that can bind to the second ligand or to said complex. For example, in case the second ligand forms part of a G-protein complex, the binding domain or binding unit in the second fusion protein can be a VHH domain that binds to said complex, for example to a subunit within said complex or to an interface between two or more of the said subunits. As mentioned herein, example of such a VHH domain is the VHH referred to as “CA4435” (SEQ ID NO:1 in WO2012/75643 and SEQ ID NO:1 herein).

The second ligand (4) may also be a synthetic or semi-synthetic analog or derivative of such a naturally occurring ligand, for example an analog or derivative with a primary amino acid sequence that differs from the primary amino acid sequence of the corresponding natural ligand by deletion, insertion and/or substitution of a limited number of amino acid residues or stretches of amino acid residues. Such analogs or derivatives may again be provided using suitable techniques of recombinant DNA technology known per se, which again in one aspect may involve expression in a suitable host or host cell of a nucleotide sequence or nucleic acid that encodes the analog or derivative (preferably, as part of the entire second fusion protein also including the second binding member (7) and any linker (11), if present). For example, when the translayer protein (2) is a 7TM or GPCR, the second ligand (4) may be an analog or derivative of G-protein (preferred), which again may have one or more amino acid differences (as defined herein) with the native sequence, provided that the analog or derivative still has sufficient affinity for the translayer protein (2) to allow the analog or derivative to be suitably used in the methods of the invention.

For example, in one specific embodiment, such an analog or derivative of a naturally occurring G-protein may be a naturally occurring G-protein in which one or more amino acid residues (and/or one or more stretches of amino acid residues) have been replaced by one or more amino acid residues (and/or one or more stretches of amino acid residues) that occur at (essentially) the same or corresponding position(s) in another naturally occurring G-protein.

Where the G-protein is a heterotrimeric protein, such a replacement of one or more amino acid residues (and/or of one or more stretches of amino acid residues) may be present or performed in any one, any two or all three of the G-alpha, G-beta and/or G-gamma subunits, and may in particular be in the G-alpha subunit.

For example, it is well known that, in humans, there are multiple genes each encoding for a different G-alpha subunits that there are multiple isoforms of G-alpha which can be grouped into different functional subfamilies (reference is for example made to Flock et al., Nature, 2015, 524(7564), 173-179; and Nehme et al., PLoS One, 2017, 12(4)), and such an analog or derivative of a naturally occurring G-alpha subunit that can be used in the present invention may be obtained by replacing one or more amino acid residues (and/or one or more stretches of amino acid residues) in (the amino acid sequence of) a naturally occurring G-alpha subunit with one or more amino acid residues (and/or one or more stretches of amino acid residues) that occur at (essentially) the same or corresponding position(s) in another naturally occurring alpha subunit (which may belong to the same subfamily or a different subfamily as the original subunit). Some specific but non-limiting examples are a naturally occurring Gα_{S} subunit in which one or more amino acid residues and/or one or more stretches of amino acid residues have been replaced with one or more amino acid residues and/or one or more stretches of amino acid residues that occur at (essentially) the same or corresponding position(s) of a Gαᵢ subunit or a naturally occurring Gα_{S} subunit in which one or more amino acid residues and/or one or more stretches of amino acid residues have been replaced with one or more amino acid residues and/or one or more stretches of amino acid residues that occur at (essentially) the same or corresponding position(s) of a Gα_{q} subunit. Often, but not exclusively, such replaced/substituted amino acids or stretches of amino acids will be present at or close to the C-terminus of the alpha-subunit.

Some specific but not limiting examples of such “chimeric” G-proteins and their design can also be found in the scientific literature Reference is for example again made to the publications by Flock et al. and by Nehme et al. cited above.

The second ligand (4) may also be another type of ligand that has been generated to bind to the binding site (9) on the translayer protein (2), and preferably binds in the manner as further described herein.

In one specifically preferred aspect, when the methods of the invention are performed in a suitable cell, the second ligand (4) is a preferably a polypeptide, protein, amino acid sequence or other chemical entity that can be obtained by suitably expressing, preferably in the cell that is used in the method of the invention, a nucleic acid or nucleotide sequence that encodes the same.

As mentioned herein, and irrespective of whether it is a naturally occurring ligand of the translayer protein (2) (such as a naturally occurring G-protein), a synthetic or semi-synthetic analog or derivative of such a naturally occurring ligand (such as a chimeric G-protein as described herein), or another kind of ligand (such as a ConfoBody as further described herein), the second ligand (4) will generally be such that it is capable to binding, and in particular specifically binding, to an epitope on the translayer protein (2), and in particular to the binding site (9). In particular, the second ligand (4) may be such that it is capable to binding, and in particular specifically binding, to an epitope that, if the translayer protein (2) were in its native cellular environment, would be an intracellular epitope.

As mentioned herein, said epitope (i.e. the binding site (9) may be a linear epitope or a conformational epitope, and is preferably a conformational epitope (as described herein). For example, when the translayer protein (2) is a GPCR, the epitope may comprise one or more amino acid residues and/or stretches of amino acid residues on at least one intracellular loop of the GPCR, and may in particular be a conformational epitope that is formed by and/or comprises one or more amino acid residues and/or stretches of amino acid residues on at least two different intracellular loops of the GPCR.

The epitope for the second ligand (4) may in particular be (part of) an epitope on the translayer protein (2) that is involved in the signaling that is mediated by the translayer protein (2). For example, the second ligand may bind to an epitope on the translayer protein (2) that lies within a binding site for a downstream signaling protein. For example, when the translayer protein (2) is a GPCR, the second ligand (4) may be a binding domain or binding unit that is capable of specifically binding to a conformational epitope that is comprised in, located at or overlaps with the G-protein binding site of a GPCR.

When the translayer protein (2) used is a protein that can take on/exists in two or more conformations (such as a basal state/conformation, an active state/conformation and/or an inactive state/conformation) and/or that can undergo a conformational change (and in particular, a functional conformational change), the second ligand (4) is preferably such that it is capable of binding, and in particular specifically binding, to a functional conformational state of the translayer protein (2).

In a specifically preferred aspect, the second ligand (4) is such that it is capable, upon binding to the translayer protein (2), of stabilizing and/or of inducing a functional and/or active conformational state of the translayer protein (2) (and/or of shifting the conformational equilibrium of the translayer protein (2) from an inactive or less active state(s) towards more active states), of bringing the translayer protein into a more druggable conformation (and/or of shifting the conformational equilibrium of the translayer protein (2) from a less druggable conformation(s) into more druggable conformation(s)), of changing the conformation of (the relevant binding pocket on) the protein so as to make it more amenable or accessible for binding of the first ligand (3) or generally increasing the affinity of the interaction between the first ligand (3) (and/or of shifting the conformational equilibrium of the protein towards such conformations) and/or of inducing and/or stabilizing the formation of a complex comprising the second ligand, the translayer protein and the first ligand (and/or of shifting the conformational equilibrium of the translayer protein (2) towards the formation of such a complex), or any combination thereof. As such, when the translayer protein (2) is a GPCR, the second ligand (4) may be such that it can bind to, and in particular stabilize and/or induce, a functional conformational state of said GPCR, more preferably of an active conformational state of said GPCR. The second ligand (4) if preferably also such that it preferentially/specifically binds the protein or GPCR when it is bound to an agonist (for example, it is bound by a first ligand (3) that acts as an agonist for the protein or GPCR) compared to conformational states in which the protein or GPCR is either not bound by any first ligand (3) or bound by a ligand (3) that acts as an inverse agonist; and/or such that it increases the affinity of the protein or GPCR for at least one compound or ligand that acts as an agonist of the protein or GPCR (i.e. at least twofold, in particularly at least fivefold, and more preferably at least tenfold).

As mentioned, one preferred class of compounds for use in the invention as the second ligand (in particular when the second ligand is included in the second fusion protein) are generally described in WO2012/007593, WO2012/007594, WO2012/75643, WO 2014/118297, WO2014/122183 and WO 2014/118297 and comprise VHH domains (Confobodies) that are capable of stabilizing a GPCR in a desired conformation.

Also, WO2012/75643 discloses a number of VHH domains that can bind indirectly to a GPCR, i.e. by binding to a G protein or a G protein complex. Some preferred but non-limiting examples of these are the VHH referred to as “CA4435” (SEQ ID NO:1 in WO2012/75643 and SEQ ID NO:1 herein) which can bind to the G-protein complex and the VHH referred to as “CA4437” (SEQ ID NO:4 in WO2012/75643 and SEQ ID NO:2 herein) which can bind to the G-protein. Such VHH domains can be suitably included in the second fusion so as to provide a second fusion protein that can bind indirectly to a GPCR by binding to the G-protein or G-protein complex.

Thus, in one preferred aspect of the invention, the second fusion protein comprises at least one such VHH or ConfoBody and the second binding member (7).

Generally, in the invention, the first binding member (6) and the second binding member (7) will come into close proximity to each other when the second fusion protein binds directly or indirectly (both as defined herein) to the translayer protein (2). In particular, the first and second binding member will come into close proximity to each other when the second ligand (4) that is present in the second fusion protein binds directly to the translayer protein (2) or when the binding domain or binding unit (5) that is present in the second fusion protein binds indirectly to the translayer protein, i.e. when said binding domain or binding unit (5) binds to the second ligand (4) or, in the case of the embodiment shown in FIG. 3, to the protein complex (12), which second ligand (4) or protein complex (12) in turn binds to or is bound by the translayer protein (2). It will be clear to the skilled person that preferably, the first and second binding member should not by themselves have a high affinity for each other, so that their association (and the concomittent generation of the detectable signal) are driven mainly by the first and second binding member coming into each other's proximity because the second ligand binds (directly or indirectly) to the translayer protein, and essentially not or only by a lesser degree by the affinity between the first and second ligand (with the NanoBiT system from Promega being an example of such a suitable binding pair). However, it should also be noted that any such affinity between the first and second ligand will generally provide a baseline for the detectable signal which should essentially not interfere with the assay of the invention as the read-out of this assay primarily looks at any changes in the detectable signal for example upon adding the first ligand to an arrangement of the invention that does not yet comprise the first ligand (more generally it should also be noted that for some uses of the methods and arrangements of the invention, it may be preferable to have some level of baseline signal, as the read-out can then also comprise a decrease in signal compared to the baseline).

Thus, generally, in the invention, the detectable signal that is generated by the first and second binding members (or any change in said signal) will be proportional to the amount of second fusion protein that is bound directly or indirectly to the translayer protein (2). This in turn will depend on the binding interaction between the second ligand (4) and the translayer protein (and in particular, between the second ligand and one or more specific conformations that the translayer protein can assume, such as a functional, active and/or druggable conformation) and/or on any changes to said binding interaction (and in particular on any changes to said binding interaction that are the result of a conformational change in the translayer protein and/or a shift in the conformational equilibrium of the translayer protein, for example due to the binding of the first ligand to the translayer protein and/or the formation of a complex between the first ligand, the translayer protein and the second ligand).

Included herein are methods of identifying and creating the various components of the above described arrangements and compositions as well as methods of assembling such arrangements and compositions. Such methods can be combined with and form part of any assays and methods for measuring or determining one or more properties of the first ligand.

By way of non-limiting example, a method for determining one or more properties of the first ligand as described herein may include one or more steps directed to determining that the second ligand: binds to translayer protein, specifically binds to the translayer protein, specifically binds to a domain of the translayer protein that is located in the second environment, is a conformation selective binding agent for the translayer protein, stabilizes a conformation of the translayer protein, stabilizes an inactive conformation of the translayer protein, stabilizes an functional, active, and/or druggable conformation of the translayer protein, and/or stabilizes a complex of the translayer protein and the first ligand

Based on this and the further disclosure herein, it will be clear to the skilled person that the methods and arrangements of the invention can be used to measure or determine one or more properties of the first ligand (and in particular, the properties of the first ligand that relate to, influence and/or determine the interaction between the first ligand and the translayer protein), one or more properties of the second ligand (and in particular, the properties of the second ligand that relate to, influence and/or determine the interaction between the second ligand and the translayer protein) and/or one or more properties of any binding domain or binding unit that is present in the second fusion protein (and in particular, when said binding domain or binding unit binds directly to the translayer protein, the properties of said binding domain or binding unit that relate to, influence and/or determine the interaction between said binding domain or binding unit and the translayer protein; or, when said binding domain or binding unit binds to the second ligand and/or a protein complex comprising the same, that relate to, influence and/or determine the interaction between said binding domain or binding unit and the second ligand or said complex).

More in particular, with respect to the first ligand, the methods and arrangements of the invention can be used to measure or determine the ability of the first ligand to bind to the translayer protein, to effect a conformational change in the translayer protein and/or to effect a change in the conformational equilibrium of the translayer protein. For example, as further described herein, the methods and arrangements of the invention can be to measure or determine the ability of a given first ligand to act as an agonist, antagonist, inverse agonist, inhibitor or modulator (such as an allosteric) modulator of the translayer protein and/or to screen for or identify small molecules, proteins or other compounds or chemical entities that act or can act as an agonist, antagonist, inverse agonist, inhibitor or modulator (such as an allosteric) modulator of the translayer protein. In this respect, it will be clear to the skilled person based on the disclosure herein that when the methods and arrangements of the invention are to be used for such a purpose (i.e. for a purpose with respect to the first ligand), that then usually (and preferably) the other elements used in the arrangement of the invention (such as the second ligand and/or any binding domain or binding unit present in the second fusion protein) will be chosen such that they have known properties (i.e. that their properties relevant to their use in the methods and the arrangements of the invention are known and/or have been characterized) and/or such that they have already been validated for use in the methods and the arrangements of the invention.

The assays of the invention can also be performed in the presence of a compound that has a known effect on the translayer protein (for example in the presence of a known agonist, antagonist, inverse agonist, inhibitor or modulator—such as an allosteric modulator—of the translayer protein), at a concentration where said “known” compound is known to have its effect on the translayer protein. Said known compound will then usually be present in the same environment as the first ligand (i.e. the ligand for which the properties are being determined using the assay of the invention). For example, in the methods of the invention in which the first ligand is added to an arrangement of the invention in which said first ligand is not yet present, the known compound may be added essentially at the same time as the first ligand, may be added separately prior to addition of the first ligand, or may be added after the first ligand may be added, and the read-out from the assay may vary depending on the order in which the first ligand and the known compound are added and, when the first ligand and the known compound are not added at essentially the same moment in time, the time between the moment that the first ligand is added and the known compound is added (or visa versa). It is also possible that, by changing the order and or timing of adding the first ligand and the known compound, it will be possible to determine different properties of the first ligand and/or to identify different first ligands having such properties.

For example and without limitation, the assays of the invention can be performed in the presence of a known agonist of the translayer protein (i.e. present in the same environment as the first ligand). In this set-up, the assay of the invention may for example be used to determine whether and how the first ligand is capable of counteracting the agonist effect of the known compound, for example, because it acts as an antagonist (so that in this set-up, the assay of the invention may be used to identify and/or characterize potential antagonists agonists of the translayer protein). The set-up with the presence of a known agonist may for example also be used to identify and/or characterize first ligands that can as an allosteric modulator that increases or decreases the effect of the agonist and/or that can act as an inverse agonist of the translayer protein. It may also be possible to perform competition assays between the first ligand and the known compound.

The methods and arrangements of the invention can be used to measure or determine the ability of the second ligand to bind to the translayer protein and in particular to bind to and/or to stabilize a particular conformation of the translayer protein (such as a functional, active or druggable conformation) and/or to stabilize and/or induce the formation of a complex between the first ligand, the second ligand and the translayer protein. For example, as further described herein, the methods and arrangements of the invention can be used to measure or determine the ability of a given VHH to act as a ConfoBody for the translayer protein or to identify, optimize or validate VHHs that can act as a ConfoBody. For this purpose, usually, the VHH or candidate VHH will be present in the second fusion protein (i.e. as the second ligand) and will bind directly to the translayer protein or be tested for its ability to bind directly to the translayer protein or one or more specific conformations of the translayer protein. As also further described herein, the methods and arrangements of the invention can also be used to measure or determine the ability of an analog, derivative or ortholog of a natural ligand of the translayer protein to act as a ligand of the translayer protein (for example, to test analogs, derivatives or orthologs of a naturally occurring G-protein to act as a ligand of the relevant GPCR). In this case, usually, the second ligand will not be present in the second fusion protein (although it is also possible to use a second fusion protein that comprises said analog, derivative or ortholog as the second ligand) but instead the second fusion protein will comprise a binding domain or binding unit (such as a VHH) that can bind to the second ligand (or a complex comprising the same). In this respect, it will be clear to the skilled person based on the disclosure herein that when the methods and arrangements of the invention are to be used for such a purpose (i.e. for a purpose with respect to the second ligand), that then usually (and preferably) the other elements used in the arrangement of the invention (such as the first ligand and/or any binding domain or binding unit present in the second fusion protein) will be chosen such that they have known properties (i.e. that their properties relevant to their use in the methods and the arrangements of the invention are known and/or have been characterized) and/or such that they have already been validated for use in the methods and the arrangements of the invention.

The methods and arrangements of the invention can also be used to measure or determine the ability of a binding domain or binding unit that is present in the second fusion protein to bind to a given second ligand and/or to a protein complex that comprises a second ligand. For example, as further described herein, the methods and arrangements of the invention can be used to measure or determine the ability of a given VHH to bind to a G-protein and/or to identify, optimize or validate such VHHs that can bind indirectly to the translayer protein (which could then for example be used as a binding domain or binding unit in an arrangement of the invention as described herein or for any other suitable purpose). Such methods and arrangements of the invention could also be used to measure or determine the ability of a given VHH to bind to a protein complex that comprises a G-protein and/or to identify, optimize or validate such VHHs (again, such VHHs could be used as a binding domain or binding unit in an arrangement of the invention as described herein or for any other suitable purpose). In this respect, it will be clear to the skilled person based on the disclosure herein that when the methods and arrangements of the invention are to be used for such a purpose (i.e. for a purpose with respect to a binding domain or binding unit that binds indirectly to the translayer protein), that then usually (and preferably) the other elements used in the arrangement of the invention (such as the first ligand and the second ligand) will be chosen such that they have known properties (i.e. that their properties relevant to their use in the methods and the arrangements of the invention are known and/or have been characterized) and/or such that they have already been validated for use in the methods and the arrangements of the invention.

In the invention, generally, the detectable signal will preferably be generated in response to, and more preferably also proportional to, a conformational change in the translayer protein and/or a shift in the conformational equilibrium of the translayer protein. As also further described herein, but again without being limited to any specific mechanism or explanation, said conformational change and/or shift in the conformational equilibrium of the translayer protein may in turn be caused by a first ligand binding to the translayer protein (or otherwise causing a conformational change in the translayer protein) and/or by the formation of a complex of the first ligand, the translayer protein and the second ligand (which second ligand may for example stabilize said complex or otherwise induce or promote the formation of said complex). Thus, more generally, in the invention, the detectable signal (or any change therein, as further described herein) will be generated in response to the presence of the first ligand in the first environment and/or in response to the first ligand binding to the translayer protein (or otherwise causing a conformational change in the translayer protein and/or a shift in the conformational equilibrium of the translayer protein).

Also, usually, and in particular when the methods and arrangements of the invention are used to test, optimize and/or validate a first ligand and/or to identify small molecules, proteins, ligands or other chemical entities that can act as an agonist, antagonist, inverse agonist, inhibitor or modulator (such as an allosteric) modulator of the translayer protein, the detectable signal (or any change therein, as further described herein) will be proportional to the amount and/or concentration of the first ligand that is present in the first environment (and/or to which the translayer protein is exposed) and/or to the affinity of the first ligand for the translayer protein (e.g. in comparison to other ligands tested).

Thus, based on the description herein, it will be clear to the skilled person that in one aspect of the invention, the methods and arrangements described herein will be used to detect the presence of, and/or to determine the amount and/or concentration of, the first ligand in the first environment. The methods and arrangements described herein may also be used to measure the amount of signal that arises when different concentrations of the first ligand are present in the first environment, for example to establish a relationship between the amount/concentration of the first ligand in the first environment and the (level of and/or change in) the detectable signal. The methods and arrangements described herein may also be used to determine the affinity of the first ligand for the translayer protein, for example by comparing the signal generated by one or more known concentrations of the first ligand in the first environment with signals generated in the same arrangement by known concentrations of other ligands with known affinity for the translayer protein.

As further described herein, the methods and arrangements of the invention may also be used to determine whether a given (first) ligand is an agonist, antagonist, inverse agonist, inhibitor or modulator (such as an allosteric) modulator of the translayer protein.

It will also be clear to the skilled person that, when the methods and arrangements of the invention are being used to determine one or more characteristics of the first ligand, that the arrangement of the invention will usually first be set-up or otherwise established without the first ligand being present, and that then subsequently the arrangement will be contacted with the ligand (e.g. by adding the ligand to the first environment), after which the detectable signal (or any change therein) that results from the presence of the first ligand will be measured (and optionally compared to the signal without the presence of the first ligand and/or with one or more reference values). Thus, the arrangements described herein without the first ligand being present (for example, before the first ligand is added) form further aspects of the invention.

Another aspect of the invention is a method for providing an arrangement of the invention as described herein, which method comprises the step of adding a first ligand to an arrangement of the invention (as described herein) that does not (yet) comprise a first ligand. The arrangement thus obtained may then be used to measure or otherwise determine at least one property of the first ligand, and in particular a property of the first ligand that can be measured or otherwise determined using the arrangement of the invention.

As will be clear to the skilled person based on the disclosure herein, an arrangement of the invention without the first ligand being present (i.e. an arrangement of the invention that does not yet comprise a first ligand) will at least comprise the following elements:
a boundary layer that separates a first environment from a second environment;
a translayer protein;
a ligand for the translayer protein that is present in the second environment; and
a binding pair that consists of at least a first binding member and a second binding member, which binding pair is capable of generating a detectable signal;
   which elements are arranged with respect to each other (and where applicable operably linked to and/or associated with each other) in the manner as further described herein (i.e. essentially in the same way as described for the arrangements of the invention that comprise the first ligand).

In particular, an arrangement of the invention without the first ligand being present (i.e. an arrangement of the invention that does not yet comprise a first ligand) will at least comprise the following elements:
a boundary layer that separates a first environment from a second environment;
a binding pair that consists of at least a first binding member and a second binding member, which binding pair is capable of generating a detectable signal;
a translayer protein that is suitably fused or linked (either directly or via a suitable linker or spacer) to one of the binding members of said binding pair (i.e. so as to form a first fusion protein); and
a second ligand for the translayer protein that is present in the second environment;
   which elements are arranged with respect to each other (and where applicable operably linked to and/or associated with each other) in the manner as further described herein (i.e. essentially in the same way as described for the arrangements of the invention that comprise the first ligand). In particular, the second member of the binding pair may be part of a second fusion protein (which is different from the first fusion protein that comprises the translayer protein and the first binding member of the binding pair), which second fusion protein is as further described herein.

More in particular, an arrangement of the invention without the first ligand being present (i.e. an arrangement of the invention that does not yet comprise a first ligand) will at least comprise the following elements:
a boundary layer that separates a first environment from a second environment;
a binding pair that consists of at least a first binding member and a second binding member, which binding pair is capable of generating a detectable signal;
a first fusion protein comprising a translayer protein and one of the binding members of said binding pair (i.e. such that said member of the binding pair is present in the second environment);
a second fusion protein comprising a protein that can bind directly or indirectly to the translayer protein and the other binding member of said binding pair, which second fusion protein is present in the second environment;
   which elements are arranged with respect to each other (and where applicable operably linked to and/or associated with each other) in the manner as further described herein (i.e. essentially in the same way as described for the arrangements of the invention that comprise the first ligand).

Other aspects, embodiment and preferences for the arrangements of the invention without the first ligand are as described herein for the arrangements of the invention with the first ligand, but then without the first ligand being present.

Generally, any such arrangement of the invention without the first ligand being present will become the corresponding arrangement of the invention with the first ligand once the first ligand is added as part of the methods described herein. Thus, another aspect of the invention is a method for providing an arrangement of the invention as described herein, which method comprises the step of adding a first ligand to an arrangement of the invention (as described herein) that does not (yet) comprise a first ligand. The arrangement thus obtained may then be used to measure or otherwise determine at least one property of the first ligand, and in particular a property of the first ligand that can be measured or otherwise determined using the arrangement of the invention.

The invention also relates to a method of measuring or otherwise determining at least one property of a compound or ligand, which method comprises at least the steps of:
adding said compound or ligand as a first ligand to an arrangement of the invention that does not yet comprise a first ligand; and
measuring or otherwise determining at least one property of said compound or ligand, in which said property is a property that can be measured or otherwise determined using said arrangement.

In this aspect of the invention, said property is preferably a property that is representative for the ability of the compound or ligand to bind to and/or to modulate the translayer protein (such as affinity).

The invention also relates to a method of measuring or otherwise determining the ability of a compound or ligand to change the detectable signal that is generated by a binding pair that is present in an arrangement of the invention as further described herein, which method comprises at least the steps of:
adding said compound or ligand as a first ligand to an arrangement of the invention that does not yet comprise a first ligand; and
determining whether adding said compound or ligand results in a change in the detectable signal that is generated by the binding pair used in said arrangement, and optionally measuring said change in said detectable signal.

Thus, in another aspect, the invention relates to a method that comprises at least the steps of:
a) providing an arrangement that at least comprises the following elements:
   a boundary layer that separates a first environment from a second environment;
   a translayer protein;
   a ligand for the translayer protein that is present in the second environment; and
   a binding pair that consists of at least a first binding member and a second binding member, which binding pair is capable of generating a detectable signal;
      in which said elements are arranged with respect to each other (and where applicable operably linked to and/or associated with each other) in the manner as further described herein;
         and;
b) adding a first ligand to the first environment.
   which method preferably further comprises the step of:
c) measuring the signal that is generated by the binding pair and/or measuring the change in the signal that is generated by the binding pair.

In a more specific aspect, the invention relates to a method that comprises at least the steps of:
a) providing an arrangement that at least comprises the following elements:
   a boundary layer that separates a first environment from a second environment;
   a binding pair that consists of at least a first binding member and a second binding member, which binding pair is capable of generating a detectable signal;
   a translayer protein that is suitably fused or linked (either directly or via a suitable linker or spacer) to one of the binding members of said binding pair (i.e. so as to form a first fusion protein); and
   a second ligand for the translayer protein that is present in the second environment; in which said elements are arranged with respect to each other (and where applicable operably linked to and/or associated with each other) in the manner as further described herein;
      and;
b) adding a first ligand to the first environment.
   which method preferably further comprises the step of:
c) measuring the signal that is generated by the binding pair and/or measuring the change in the signal that is generated by the binding pair.

In another specific aspect, the invention relates to a method that comprises at least the steps of:
a) providing an arrangement that at least comprises the following elements:
   a boundary layer that separates a first environment from a second environment;
   a binding pair that consists of at least a first binding member and a second binding member, which binding pair is capable of generating a detectable signal;
   a first fusion protein comprising a translayer protein and one of the binding members of said binding pair (i.e. such that said member of the binding pair is present in the second environment);
   a second fusion protein comprising a protein that can bind directly or indirectly to the translayer protein and the other binding member of said binding pair, which second fusion protein is present in the second environment;
   in which said elements are arranged with respect to each other (and where applicable operably linked to and/or associated with each other) in the manner as further described herein;
      and;
b) adding a first ligand to the first environment.
   which method preferably further comprises the step of:
c) measuring the signal that is generated by the binding pair and/or measuring the change in the signal that is generated by the binding pair.

As further described herein, in this aspect of the invention, said first ligand can be any desired and/or suitable compound or ligand, including but not limited to small molecules, small peptides, biological molecules or other chemical entities. It will also be clear to the skilled person that the method according to this aspect (and the other methods of the invention) can be used to measure or otherwise determine at least one property of the compound or ligand that is added to the arrangement as the first ligand, and in particular to measure or otherwise determine the ability of said compound or ligand to give rise to a change in the detectable signal that is generated by the binding pair, the ability of said compound or ligand to bind to the translayer protein, the ability of said compound or ligand to effect a conformational change in the translayer protein, and/or the ability of said compound or ligand to modulate (as defined herein) the translayer protein and/or the signaling pathway(s) and/or biological mechanism(s) in which the translayer protein is involved. In particular, said methods can be used to determine whether such a compound or ligand is or can act as an agonist, antagonist, inverse agonist, inhibitor or modulator (such as an allosteric modulator) of the translayer protein and/or the signaling pathway(s) and/or biological mechanism(s) in which the translayer protein is involved. Also, the methods and arrangements of the invention can be used to identify and/or screen for compounds or ligands that have the ability to give rise to a change in the detectable signal that is generated by the binding pair, the ability to bind to the translayer protein, the ability to effect a conformational change in the translayer protein, the ability to modulate the translayer protein and/or the signaling pathway(s) and/or biological mechanism(s) in which the translayer protein is involved, and/or the ability to act as an agonist, antagonist, inverse agonist, inhibitor and/or modulator (such as an allosteric modulator) of the translayer protein, and such uses of the methods and arrangements described herein form further aspects of the invention.

It should also be noted that in a further aspect, the methods and arrangements of the invention can also be used to measure or otherwise determine at least one property of the second ligand, such as the ability of the second ligand to bind to the translayer protein, the ability of the second ligand to bind to and/or to stabilize a specific conformation of the translayer protein (such as an active and/or druggable conformation), and/or the ability of the second ligand to stabilize a complex of the translayer protein, the first ligand and the second ligand. Usually, in this aspect of the invention, one or more first ligands with known ability to bind to and/or to modulate the translayer protein will be used to determine whether an arrangement of the invention that comprises the (candidate) second ligand will give rise to a detectable signal when said first ligand is added to said arrangement (e.g. in one or more known concentrations).

For example, this aspect of the invention can be used to identify or optimize binding domains or binding units (such as an ISVD) that can bind directly (as defined herein) to the translayer protein, and in particular binding domains or binding units that are specific and/or selective for the conformation of the translayer protein that arises when the first ligand used binds to the translayer protein. The binding domains or binding units so identified, optimized and/or validated can for example be used in an arrangement of the invention (i.e. as part of the second fusion protein) and/or be used to induce or stabilize a specific conformation of the translayer protein (for example for screening or crystallisation purposes, as described in the prior art cited herein for conformation-specific ligands of GPCRs). Thus, for example, this aspect of the invention can be used to identify, optimize and/or validate ISVDs for use as ConfoBodies, which can then be used for the purposes described herein and/or for uses known per se for conformation-specific ISVDs. Reference is again made to the further prior art cited herein.

Generally, in this aspect of the invention, the binding domain, binding unit, ligand or other protein that is to be tested or validated for binding (directly) to the translayer protein will be part of the second fusion protein. Thus, the invention further relates to a method of measuring or otherwise determining at least one property of a binding domain, binding unit, ligand or other protein, which method comprises at least the steps of:
providing an arrangement of the invention that does not yet comprise the first ligand, in which the second fusion protein comprises said binding domain, binding unit, ligand or other protein;
adding a first ligand to said arrangement; and
determining whether adding said first ligand results in a change in the detectable signal that is generated by the binding pair used in said arrangement, and optionally measuring said change in said detectable signal.

As will be clear to the skilled person based on the disclosure herein, said at least one property of said binding domain, binding unit, ligand or other protein will in particular be the ability of said binding domain, binding unit, ligand or other protein to bind to the translayer protein (and in particular to the conformation that the translayer protein assumes when the first ligand used binds to the translayer protein), the ability of said binding domain, binding unit, ligand or other protein to stabilize the conformation that the translayer protein assumes when the first ligand used binds to the translayer protein, and/or the ability of said binding domain, binding unit, ligand or other protein to promote or induce the formation of a complex of the first ligand used, the translayer protein and said binding domain, binding unit, ligand or other protein and/or the ability of said binding domain, binding unit, ligand or other protein to stabilize such a complex.

In another aspect of the invention, the arrangements described herein are again used to measure or otherwise determine at least one property of the second ligand and/or to identify, optimize and or validate a candidate second ligand, but in this aspect the second fusion protein will not comprise the second ligand to be tested or the candidate second ligand, but will instead comprise a binding domain or binding unit that is known to bind to the second ligand to be tested or the candidate second ligand. In other words, in this aspect, the second fusion protein will comprise a binding domain or binding unit that can bind indirectly (as defined herein) to the translayer protein, i.e. via the second ligand to be tested or the candidate second ligand or via a protein complex comprising the same, provided said second ligand or complex is capable of binding to the translayer protein (and in particular, to conformation of the translayer protein that arises when the first ligand binds to the translayer protein). As with the previous aspects, this aspect can also be used to identify, optimize and/or validate (candidate) ligands for the translayer protein, for example ligands that are synthetic or semi-synthetic analogs or derivatives of a naturally occurring ligand of the translayer protein. For example, when the translayer protein is a GPCR, this aspect of the invention can be used to identify, optimize and/or validate analogs or derivatives of the G-protein that is the native ligand of said GPCR, or to determine whether an ortholog of the naive G-protein of the relevant GPCR is capable of binding to said GPCR and/or to stabilize a complex of the GPCR and the first ligand used.

Thus, the invention further relates to a method of measuring or otherwise determining at least one property of a ligand or other protein, which method comprises at least the steps of:
providing an arrangement of the invention that does not yet comprise the first ligand, in which said ligand or other protein is present and/or is used as the second ligand, and in which the second fusion protein comprises a binding domain or binding unit that can bind to said ligand or other protein and/or to a protein complex comprising said ligand or other protein;
adding a first ligand to said arrangement; and
determining whether adding said first ligand results in a change in the detectable signal that is generated by the binding pair used in said arrangement, and optionally measuring said change in said detectable signal.

As described herein, in one specific aspect of the invention, the methods of the invention are performed using a suitable cell or cell line in which all of the elements of an arrangement of the invention are suitably present and arranged so as to provide an operable arrangement of the invention. Such a cell or cell line will suitably comprise the translayer protein (2) in its cell wall or cell membrane, i.e. such that the translayer protein (2) is present in and spans the cell wall or cell membrane of the cell such that at least one part of the amino acid sequence of the translayer protein extends out (as defined herein) into the extracellular environment and at least one of other part of the amino acid sequence of the translayer protein extends out (as defined herein) into the intracellular environment. Also, preferably and as further described herein, the translayer protein (2) will form part of the first fusion protein as described herein and the arrangement will also comprise a second fusion protein as described herein. More preferably, the extracellular environment will be the “first environment” (i.e. the environment in which the first ligand (3) is present or to which the first ligand (3) is added) and the intracellular environment will be the “second environment” (i.e. the environment in which the binding pair (6/7) and the second fusion protein are present).

Thus, in a further aspect, the invention relates to a method or arrangement as described herein, in which the boundary layer (2) is the wall or the membrane of cell.

As also described herein, when the methods of the invention are performed in cells or in a suitable cell line, the cell or cell line used is preferably such that it suitably expresses one or more, and preferably all, of the following elements of the arrangement of the invention:
the first fusion protein comprising the translayer protein (2) and the first binding member (6);
the second fusion protein comprising the second binding member (7) and a protein that can bind directly or indirectly (as defined herein) to the translayer protein (2);
   and/or
when the second fusion protein binds indirectly to the translayer protein (2), the second ligand (4) and/or the proteins that make up the protein complex (12)

In the context of a cell or cell line that expresses one or more elements of an arrangement of the invention, and more generally in the context of the present description and claims, with the term “suitably expresses” is meant that the cell or cell line expresses or is capable of expressing (i.e. under the conditions used for performing the methods of the invention) a nucleotide sequence or nucleic acid that encodes said element such that, when such element is expressed, it is capable of functioning as an operable part of the arrangement of the invention. For example, with respect to the translayer protein (2), this means that the translayer protein is expressed as part of the first fusion protein such that the expressed translayer protein (2) becomes suitably anchored or otherwise incorporated into the cell wall or cell membrane of the cell such that it spans the cell wall or cell membrane with at least one part of the amino acid sequence of the translayer protein extending out (as defined herein) into the extracellular environment and at least one of other part of the amino acid sequence of the translayer protein extending out (as defined herein) into the intracellular environment. With respect to the first and second fusion protein, “suitably expresses” means that the first and second fusion protein are expressed such (and most preferably expressed in the intracellular environment such) that the first and second binding members of the binding pair (6/7) can come into contact or in close proximity to each other when the second fusion protein binds directly or indirectly to the translayer protein (2), in the manner as further described herein.

Any suitable expression of each such element of an arrangement of the invention can be transient or constitutive expression, as long as all the required elements of the arrangement of the invention are suitably and operably present in sufficient amounts at the point in time when the cell is to be used for performing the method of the invention.

In one aspect of the invention, in case of an embodiment of the invention in which the second fusion protein binds indirectly to the translayer protein (i.e. where the second ligand (4) is not part of the second fusion protein), the cell or cell line used is preferably such that it natively expresses the second ligand (4) and/or the proteins that make up the protein complex (12). For example and without limitation, in this aspect of the invention, when the translayer protein (2) is a GPCR, the second ligand (4) may be a G protein that is natively expressed by the cell or cell line used and/or the protein complex (12) may be a G-protein trimer comprising a G-alpha subunit, a G-beta subunit and a G-gamma subunit that are natively expressed by the cell or cell line used. More generally, in these aspects of the invention, the cell or cell line used may be a cell or cell line that natively expresses one or more natural ligands (and in particular intracellular ligands) of the translayer protein (2) and/or that natively expresses one or more ligands that can function as a second ligand for the translayer protein (2), depending on the translayer protein (2) that is being used or screened.

The cell or cell line can be any cell or cell line that suitable for use in the methods and arrangements of the invention, including but not limited to mammalian cells and insect cells. Some preferred but non-limiting examples are human cell lines such as HEK 293 T.

Suitable techniques for transiently or stably expressing a desired protein in such a cell or cell line such that the translayer protein (2) becomes suitably anchored into the cell wall or cell membrane of said cells will be clear to the skilled person and for example include techniques involving the use of a suitable transfection reagent such as X-tremeGENE™ from SigmaAldrich or polyethylenimine (PEI).

When the invention is performed using a cell or cell line that suitably expresses one or more elements of an arrangement of the invention, the method of the invention will generally also include a step of cultivating or maintaining said cell under conditions such that said cell or cell line suitably expresses said elements.

Thus, in another aspect, the invention relates to a cell or cell line that comprises a fusion protein, said fusion protein comprising a translayer protein (as described herein) that is fused, directly or via a suitable linker, to a binding domain or binding unit that is a first binding member of a binding pair, said binding pair comprising at least said binding domain or binding unit as a first binding member and a further binding domain or binding unit as a second binding member, in which said first and second binding members of said binding pair are such that they are capable of generating a detectable signal when they come into contact with each other or into close proximity to each other. The invention also relates to a cell or cell line that expresses or is capable of expressing (i.e. under suitable conditions) such a fusion protein.

Such a cell or cell line can be as further described herein, and is preferably such that it expresses or is capable of expressing said fusion protein in such a way that the translayer protein becomes incorporated into the cell wall or cell membrane of the cell or cell line and spans said cell wall or cell membrane, more preferably such that at least one part of the amino acid sequence of the translayer protein extends out (as defined herein) into the extracellular environment and at least one other part of the amino acid sequence of the translayer protein extends out (as defined herein) into the intracellular environment. More preferably, in said cell or cell line, the first binding member of the binding pair is present in (as defined herein) the intracellular environment of the cell and/or the cell or cell line is such that it expresses or is capable of expressing the fusion protein such that, upon such expression, the first binding member is present in (as defined herein) the intracellular environment of the cell.

Also, the translayer protein that is present in the fusion protein is preferably as further described herein, and more preferably has at least two ligand binding sites, one of which extends out (as defined herein) into the extracellular environment and one of which extends out (as defined herein) into the intracellular environment. Further, as described herein, the translayer protein is preferably such that it is capable of undergoing a conformational change from one of its conformations into another conformation (and in particular, a conformational change from an essentially inactive or less active conformation into an active or more active conformation) upon binding of a ligand to a ligand binding site on the translayer protein, and in particular upon a ligand that is present in the extracellular environment binding to a ligand binding site on the translayer protein that is present in (as defined herein) the extracellular environment. As also further described herein, the translayer protein is preferably further such that it can be stabilized in an functional and/or active (or more active) conformation (and in particular in a druggable conformation and/or in a ligand-bound conformation, and more in particular in an agonist-bound conformation) by a suitable ligand, binding domain or binding unit (such as a ConfoBody as described herein or a natural ligand of the translayer protein, such as a natural intracellular ligand) binding to an intracellular binding site on the translayer protein (which can be a binding site on the translayer protein that is intracellular binding site when the translayer protein is in its native environment and/or be a binding site on the translayer protein that is intracellular binding site when the translayer protein is present in the cell or cell line that is used in the invention, and is preferably both). In particular, as also described herein, the translayer protein may be capable of forming a complex when a first ligand binds to the extracellular binding site and a second ligand binds to the intracellular binding site. More in particular, as described herein, the translayer protein may be capable of forming a complex in which the translayer protein is in a functional or active conformation that is induced by a first ligand binding to the extracellular binding site, in which said active or functional conformation is stabilized by the binding of a second ligand to the intracellular binding site, which second ligand is capable of stabilizing said functional, active or ligand-bound conformation and/or said complex. In one preferred but not-limiting aspect, the translayer protein is a transmembrane protein and in particular a 7TM. Also, the members of the binding pair and any linkers used can be as further described herein.

In another aspect, the invention relates to a cell or cell line that comprises a fusion protein, said fusion protein comprising a protein that can bind (directly or indirectly, as described herein) to a translayer protein (as described herein), which protein is fused, directly or via a suitable linker, to a binding domain or binding unit that is a binding member of a binding pair, said binding pair comprising at least a first binding member and said binding domain or binding unit as a second binding member, in which said first and second binding members of said binding pair are such that they are capable of generating a detectable signal when they come into contact with each other or into close proximity to each other. The invention also relates to a cell or cell line that expresses or is capable of expressing (i.e. under suitable conditions) such a fusion protein.

The protein that is present in said fusion protein and that can bind to the translayer protein is preferably as further described herein for the protein that can be present in the second fusion protein. Also, the members of the binding pair and any linkers used can be as further described herein. As also described herein, said protein can bind directly (as described herein) or indirectly (as described herein) to the translayer protein. Again, in this aspect, the translayer protein to which said protein can bind is preferably also as further described herein, and can in particular be a transmembrane protein and more in particular a 7TM.

As described herein, when the protein that is present in said fusion protein binds directly to the translayer protein, it is preferably such that it specifically binds to one or more functional, active and/or druggable conformations of the translayer protein, such that it induces the formation of and/or stabilizes one or more functional, active and/or druggable conformations of the translayer protein (and/or shifts the conformational equilibrium of the translayer protein towards one or more such conformations); and/or such that it induces the formation of and/or stabilizes a complex of said protein, the translayer protein and a further ligand of the translayer protein (all as further described herein). Also, when the protein that is present in said fusion protein binds directly to the translayer protein, the protein is preferably such that it can bind to an intracellular binding site on the translayer protein. Said intracellular binding site on the translayer protein can be a binding site on the translayer protein that is intracellular binding site when the translayer protein is in its native environment and/or be a binding site on the translayer protein that is intracellular binding site when the translayer protein is present in the cell or cell line that is used in the invention (and is preferably both).

Also, when the protein that is present in said fusion protein binds directly to the translayer protein, it is preferably a VHH domain or a binding domain or binding unit that is derived from a VHH domain, and in particular a ConfoBody (as described herein).

As also described herein, when the protein that is present in said fusion protein binds indirectly to the translayer protein, it is preferably such that is can bind to a ligand that can bind to the translayer protein. Said ligand can be as described herein for the “second ligand” when said second ligand does not form part of the second fusion protein. Again, said ligand is preferably such that it specifically binds to one or more functional, active and/or druggable conformations of the translayer protein, such that it induces the formation of and/or stabilizes one or more functional, active and/or druggable conformations of the translayer protein (and/or shifts the conformational equilibrium of the translayer protein towards one or more such conformations); and/or such that it induces the formation of and/or stabilizes a complex of said ligand, the translayer protein and a further ligand of the translayer protein (all as further described herein). Also, said ligand is preferably such that it can bind to an intracellular binding site on the translayer protein. Said intracellular binding site on the translayer protein can be a binding site on the translayer protein that is intracellular binding site when the translayer protein is in its native environment and/or be a binding site on the translayer protein that is intracellular binding site when the translayer protein is present in the cell or cell line that is used in the invention (and is preferably both). Also, as described herein, said ligand can also be part of a protein complex that can bind to the translayer protein (i.e. to an intracellular binding site on the translayer protein), in which case the protein that is present in the fusion protein can also bind to said protein complex.

Also, when the protein that is present in said fusion protein binds indirectly to the translayer protein, it is preferably a VHH domain or a binding domain or binding unit that is derived from a VHH domain. Also, in a preferred aspect, when the protein that is present in said fusion protein binds indirectly to the translayer protein, and said translayer protein is a GPCR, the ligand binding to the GPCR is a G-protein and the protein that is present in said fusion protein is capable of specifically binding to said G-protein or to a G-protein complex such as a G-protein trimer that comprises a G-alpha subunit, a G-beta subunit and a G-gamma subunit). Also, said G-protein may be native to the cell or cell line used or may be a suitable analog or derivative (as described herein, and recombinantly expressed in said cell or cell line) of a natural G-protein or a suitable ortholog of the G-protein that is native to the cell or cell line used (again, recombinantly expressed in the cell or cell line used).

Irrespective of whether the protein that is present in said fusion protein binds directly or indirectly to the translayer protein, the cell or cell line is preferably such that it expresses or is capable of expressing said fusion protein in the intracellular environment. Another aspect of the invention relates to such a cell or cell line that comprises such fusion protein in its intracellular environment.

In another aspect, the invention relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair.

The invention also relates to a cell or cell line that expresses or is capable of expressing (i.e. under suitable conditions) such first and second fusion proteins.

The invention in particular relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair; and
the first and second binding members of the binding pair can come into contact or in close proximity to each other when the second fusion protein binds (directly or indirectly, as described herein) to the translayer protein that forms part of the first fusion protein.

The invention also relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair; and
the first and second binding members of the binding pair are present in (as defined herein) the intracellular environment of the cell.

The invention further relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair; and
said cell or cell line is capable of generating a detectable signal (and in particular, a detectable signal that is generated by the first and second binding members of the binding pair) when the second fusion protein binds (directly or indirectly, as described herein) to the translayer protein that forms part of the first fusion protein.

The invention further relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair; and
said cell or cell line gives rise to a detectable signal and/or to a change in a detectable signal (and in particular, to a detectable signal that is generated by the first and second binding members of the binding pair and/or to a change in such a signal) when a ligand for the translayer protein that is present in the extracellular environment binds to the translayer protein.

In a particular aspect, the invention relates to a cell or cell line that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair; and
said cell or cell line gives rise to a detectable signal and/or to a change in a detectable signal (and in particular, to a detectable signal that is generated by the first and second binding members of the binding pair and/or to a change in such a signal) when an agonist for the translayer protein that is present in the extracellular environment binds to the translayer protein.

Again, such cells or cell lines that comprise or express such first and second fusion proteins can be as further described herein, and are preferably such that they expresses or are capable of expressing said first fusion protein in such a way that the translayer protein becomes incorporated into the cell wall or cell membrane of the cell or cell line and spans said cell wall or cell membrane, more preferably such that at least one part of the amino acid sequence of the translayer protein extends out (as defined herein) into the extracellular environment and at least one other part of the amino acid sequence of the translayer protein extends out (as defined herein) into the intracellular environment.

Said cells or cell lines are also preferably such that they expresses or are capable of expressing the first and second fusion protein such that, upon such expression, the first and second binding members of the binding pair can come into contact or in close proximity to each other when the second fusion protein binds (directly or indirectly, as described herein) to the translayer protein that forms part of the first fusion protein. It will be clear to the skilled person that this generally means that such cells or cell lines will express the first and second fusion proteins in such a way that, upon such expression, the first and second binding members of the binding pair will be present in (as defined herein) the same environment relative to the wall or membrane of the cell. Preferably, the cells or cell lines are such that they express or are capable of expressing the first and second fusion protein such that, upon such expression, the first and second binding members of the binding pair will both be present in (as defined herein) the intracellular environment of the cell. This also generally means that the cells or cell lines are preferably such that they expresses or are capable of expressing the second fusion protein in their intracellular environment.

Again, in the aspects of the invention that relate to cells or cell lines that express or are capable of expressing such a first and second fusion protein, the translayer protein, the protein that can bind directly or indirectly to the translayer protein, the members of the binding pair and any linkers used can all be as further described herein.

In further aspects, the invention also relates to methods, and in particular assay methods or screening methods, that involve the use of the cells or cell lines described herein. As further described herein, such assay and screening methods can in particular be used to identify compounds and other chemical entities that bind to (and in particular specifically bind to) the translayer protein, that can modulate the translayer protein and/or that modulate the signaling, signaling pathway and/or biological or physiological activitie(s) in which the translayer protein, its signaling and/or its signaling pathway is involved. As such, the cells and cell lines described herein can be used in methods to identify compounds or other chemical entities that can act as an agonist, antagonist, inverse agonist, inhibitor or modulator (such as an allosteric) modulator of the translayer protein.

The invention also relates to uses of the cells or cell lines described herein, in particular in assay and screening methods and techniques. Such methods and uses can again be as further described herein for methods and uses of the arrangements of the invention, and will generally also include a step of cultivating or maintaining said cell under conditions such that said cell or cell line suitably expresses the desired fusion protein or proteins.

Again, in all these aspects, such cells, cell lines and uses thereof are preferably as further described herein.

In another aspect of the invention, the methods of the invention are performed using a suitable liposome or vesicle in which all of the elements of an arrangement of the invention are suitably present and arranged so as to provide an operable arrangement of the invention. Such a liposome or vesicle will suitably comprise the translayer protein (2) in its wall or membrane, i.e. such that the translayer protein (2) is present in and spans the wall or membrane of the liposome or vesicle such that at least one part of the amino acid sequence of the translayer protein extends out (as defined herein) into the environment outside of the liposome or vesicle and at least one of other part of the amino acid sequence of the translayer protein extends out (as defined herein) into the environment inside the liposome or vesicle. Also, preferably and as further described herein, in aspects of the invention that are performed in a liposome or vesicle, the environment outside of the liposome or vesicle will be the “first environment” (i.e. the environment in which the first ligand (3) is present or to which the first ligand (3) is added) and the environment inside the liposome or vesicle will be the “second environment” (i.e. the environment in which the binding pair (6/7) and the second fusion protein are present).

Thus, in a further aspect, the invention relates to a method or arrangement as described herein, in which the boundary layer (2) is the wall or the membrane of a liposome or other (suitable) vesicle.

As also described herein, when the methods of the invention are performed in a liposome or vesicle, the liposome or vesicle is preferably such that it suitably contains (i.e. in such a manner as to provide an operable arrangement of the invention) the following elements of the arrangement of the invention:
the first fusion protein comprising the translayer protein (2) and the first binding member (6);
the second fusion protein comprising the second binding member (7) and a protein that can bind directly or indirectly (as defined herein) to the translayer protein (2);
   and/or
when the second fusion protein binds indirectly to the translayer protein (2), the second ligand (4) and/or the proteins that make up the protein complex (12)

Liposomes or vesicles that contain said elements can generally be provided by forming the liposomes or vesicles in the presence of the relevant elements of the arrangement of the invention, such that said elements are suitably incorporated into the liposomes of vesicles. This can generally be performed by methods and techniques known per se for forming liposomes or vesicles, preferably in a suitable aqueous buffer or another suitably aqueous medium. Such methods may also comprise a step of separating liposome or vesicles in which the elements of the desired arrangement of the invention are suitably and operably included from vesicles or liposomes that do not contain all the required elements of the arrangements and/or in which the elements do not form an operable arrangement of the invention. The elements of the arrangements that are incorporated into the liposome of vesicle can be provided in a manner known per se, for example by recombinant expression is a suitable host cell or host organism followed by isolating and purifying the expressed elements thus obtained.

Generally, in the aspects of the invention that are performed in liposomes or vesicles, where the second ligand does not form part of the second fusion protein, a sufficient amount of the second ligand should also be provided and suitably included into the vesicle or liposome.

The liposome or vesicle can be any liposome or vesicle that suitable for use in the methods and arrangements of the invention, including but not limited to liposomes based on 1,2-dioleoyl-sn-glycero-3-phosphocholine (DOPC), dioleoylphosphatidylethanolamine (DOPE) or 1-palmitoyl-2-oleoyl-sn-glycero-3-phosphocholine (POPC). The liposomes and vesicles can also be liposomes or vesicles that contain and/or are based on (e.g. reconstituted from) one or more membrane fractions obtained from cells that express the desired element(s) of the arrangement of the invention.

Thus, in another aspect, the invention relates to a liposome or vesicle that comprises a fusion protein, said fusion protein comprising a translayer protein (as described herein) that is fused, directly or via a suitable linker, to a binding domain or binding unit that is a first binding member of a binding pair, said binding pair comprising at least said binding domain or binding unit as a first binding member and a further binding domain or binding unit as a second binding member, in which said first and second binding members of said binding pair are such that they are capable of generating a detectable signal when they come into contact with each other or into close proximity to each other. The invention also relates to method for providing such a liposome or vesicle which method comprises at least the step of incorporating such a fusion protein into a liposome or vesicle and/or of forming a liposome or vesicle in the presence of said fusion protein.

As further described herein, said liposome or vesicle is preferably such that the translayer protein is anchored or otherwise suitably incorporated into the wall or membrane of the liposome or vesicle and spans said wall or membrane, more preferably such that at least one part of the amino acid sequence of the translayer protein extends out (as defined herein) into the environment outside of the liposome or vesicle and at least one other part of the amino acid sequence of the translayer protein extends out (as defined herein) into the environment inside the liposome or vesicle. More preferably, the first binding member of the binding pair is present in (as defined herein) the environment inside liposome or vesicle,

Also, the translayer protein that is present in the fusion protein is preferably as further described herein, and more preferably has at least two ligand binding sites, one of which extends out (as defined herein) into the environment outside of the liposome or vesicle and one of which extends out (as defined herein) into the environment inside the liposome or vesicle. Further, as described herein, the translayer protein is preferably such that it is capable of undergoing a conformational change from one of its conformations into another conformation (and in particular, a conformational change from an essentially inactive or less active conformation into an active or more active conformation) upon binding of a ligand to a ligand binding site on the translayer protein, and in particular upon a ligand that is present in the environment outside of the liposome or vesicle binding to a ligand binding site on the translayer protein that is present in (as defined herein) the environment outside of the liposome or vesicle. As also further described herein, the translayer protein is preferably further such that it can be stabilized in an functional and/or active (or more active) conformation (and in particular in a druggable conformation and/or in a ligand-bound conformation, and more in particular in an agonist-bound conformation) by a suitable ligand, binding domain or binding unit (such as a ConfoBody as described herein or a natural ligand of the translayer protein) binding to an intracellular binding site on the translayer protein (which can be a binding site on the translayer protein that is intracellular binding site when the translayer protein is in its native environment and/or be a binding site on the translayer protein that is present in the environment inside the liposome or vesicle when the translayer protein is present in the liposome or vesicle that is used in the invention, and is preferably both). In particular, as also described herein, the translayer protein may be capable of forming a complex when a first ligand binds to the binding site that is present in (as defined herein) the environment outside of the liposome or vesicle and a second ligand binds to the binding site that is present in (as defined herein) the environment inside the liposome or vesicle. More in particular, as described herein, the translayer protein may be capable of forming a complex in which the translayer protein is in a functional or active conformation that is induced by a first ligand binding to the binding site that is present in (as defined herein) the environment outside of the liposome or vesicle, in which said active or functional conformation is stabilized by the binding of a second ligand to the binding site that is present in (as defined herein) the environment inside the liposome or vesicle, which second ligand is capable of stabilizing said functional, active or ligand-bound conformation and/or said complex. In one preferred but not-limiting aspect, the translayer protein is a transmembrane protein and in particular a 7TM. Also, the members of the binding pair and any linkers used can be as further described herein.

In another aspect, the invention relates to a liposome or vesicle that comprises a fusion protein, said fusion protein comprising a protein that can bind (directly or indirectly, as described herein) to a translayer protein (as described herein), which protein is fused, directly or via a suitable linker, to a binding domain or binding unit that is a binding member of a binding pair, said binding pair comprising at least a first binding member and said binding domain or binding unit as a second binding member, in which said first and second binding members of said binding pair are such that they are capable of generating a detectable signal when they come into contact with each other or into close proximity to each other. The invention also relates to method for providing such a liposome or vesicle which method comprises at least the step of incorporating such a fusion protein into a liposome or vesicle and/or of forming a liposome or vesicle in the presence of said fusion protein.

The protein that is present in said fusion protein and that can bind to the translayer protein is preferably as further described herein for the protein that can be present in the second fusion protein. Also, the members of the binding pair and any linkers used can be as further described herein. As also described herein, said protein can bind directly (as described herein) or indirectly (as described herein) to the translayer protein. Again, in this aspect, the translayer protein to which said protein can bind is preferably also as further described herein, and can in particular be a transmembrane protein and more in particular a 7TM.

As described herein, when the protein that is present in said fusion protein binds directly to the translayer protein, it is preferably such that it specifically binds to one or more functional, active and/or druggable conformations of the translayer protein, such that it induces the formation of and/or stabilizes one or more functional, active and/or druggable conformations of the translayer protein (and/or shifts the conformational equilibrium of the translayer protein towards one or more such conformations); and/or such that it induces the formation of and/or stabilizes a complex of said protein, the translayer protein and a further ligand of the translayer protein (all as further described herein). Also, when the protein that is present in said fusion protein binds directly to the translayer protein, the protein is preferably such that it can bind to a binding site on the translayer protein that is an intracellular binding site when the translayer protein is in its native environment and/or a binding site on the translayer protein that is present in (as defined herein) in the environment inside the liposome or vesicle when the translayer protein is present in the liposome or vesicle when said liposome or vesicle is being used in the invention (and preferably both).

Also, when the protein that is present in said fusion protein binds directly to the translayer protein, it is preferably a VHH domain or a binding domain or binding unit that is derived from a VHH domain, and in particular a ConfoBody (as described herein).

As also described herein, when the protein that is present in said fusion protein binds indirectly to the translayer protein, it is preferably such that is can bind to a ligand that can bind to the translayer protein. Said ligand can be as described herein for the “second ligand” when said second ligand does not form part of the second fusion protein. Again, said ligand is preferably such that it specifically binds to one or more functional, active and/or druggable conformations of the translayer protein, such that it induces the formation of and/or stabilizes one or more functional, active and/or druggable conformations of the translayer protein (and/or shifts the conformational equilibrium of the translayer protein towards one or more such conformations); and/or such that it induces the formation of and/or stabilizes a complex of said ligand, the translayer protein and a further ligand of the translayer protein (all as further described herein). Also, said ligand is preferably such that it can bind to a binding site on the translayer protein that is an intracellular binding site when the translayer protein is in its native environment and/or a binding site on the translayer protein that is present in (as defined herein) in the environment inside the liposome or vesicle when the translayer protein is present in the liposome or vesicle when said liposome or vesicle is being used in the invention (and preferably both). Also, as described herein, said ligand can also be part of a protein complex that can bind to the translayer protein, in which case the protein that is present in the fusion protein can also bind to said protein complex.

Also, when the protein that is present in said fusion protein binds indirectly to the translayer protein, it is preferably a VHH domain or a binding domain or binding unit that is derived from a VHH domain. Also, in a preferred aspect, when the protein that is present in said fusion protein binds indirectly to the translayer protein, and said translayer protein is a GPCR, the ligand binding to the GPCR is a G-protein and the protein that is present in said fusion protein is capable of specifically binding to said G-protein or to a G-protein complex such as a G-protein trimer that comprises a G-alpha subunit, a G-beta subunit and a G-gamma subunit).

Irrespective of whether the protein that is present in said fusion protein binds directly or indirectly to the translayer protein, said fusion protein is preferably present in (as defined herein) the environment inside the liposome or vesicle. Also, when the second ligand does not form part of said fusion protein, the environment inside the liposome or vesicle will also contain a suitable amount of the second ligand.

In another aspect, the invention relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair.

The invention also relates to method for providing such a liposome or vesicle which method comprises at least the step of incorporating said fusion proteins into a liposome or vesicle and/or of forming a liposome or vesicle in the presence of said fusion proteins.

The invention in particular relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair; and
the first and second binding members of the binding pair can come into contact or in close proximity to each other when the second fusion protein binds (directly or indirectly, as described herein) to the translayer protein that forms part of the first fusion protein.

Again, the invention also relates to method for providing such a liposome or vesicle which method comprises at least the step of incorporating said fusion proteins into a liposome or vesicle and/or of forming a liposome or vesicle in the presence of said fusion proteins.

The invention also relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair; and
the first and second binding members of the binding pair are present in (as defined herein) the environment inside the liposome or vesicle.

Again, the invention also relates to method for providing such a liposome or vesicle which method comprises at least the step of incorporating said fusion proteins into a liposome or vesicle and/or of forming a liposome or vesicle in the presence of said fusion proteins.

The invention further relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair; and
said liposome or vesicle is capable of generating a detectable signal (and in particular, a detectable signal that is generated by the first and second binding members of the binding pair) when the second fusion protein binds (directly or indirectly, as described herein) to the translayer protein that forms part of the first fusion protein.

Again, the invention also relates to method for providing such a liposome or vesicle which method comprises at least the step of incorporating said fusion proteins into a liposome or vesicle and/or of forming a liposome or vesicle in the presence of said fusion proteins.

The invention further relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair; and
said liposome or vesicle gives rise to a detectable signal and/or to a change in a detectable signal (and in particular, to a detectable signal that is generated by the first and second binding members of the binding pair and/or to a change in such a signal) when a ligand for the translayer protein that is present in the environment outside of the liposome or vesicle binds to the translayer protein.

Again, the invention also relates to method for providing such a liposome or vesicle which method comprises at least the step of incorporating said fusion proteins into a liposome or vesicle and/or of forming a liposome or vesicle in the presence of said fusion proteins.

In a particular aspect, the invention relates to a liposome or vesicle that comprises a first fusion protein and a second fusion protein, in which:
said first fusion protein comprises a binding domain or binding unit that is a first binding member of a binding pair and said second fusion protein comprises a binding domain or binding unit that is a second binding member of said binding pair, in which said first and second binding members of said binding pair are such that they are capable of generating a detectable signal when they come into contact with each other or into close proximity to each other; and
said first fusion protein comprises a translayer protein (as described herein) that is fused, directly or via a suitable linker, to said first binding member of the binding pair; and
said second fusion protein comprises a protein that can bind (directly or indirectly, as described herein) to said translayer protein, which protein is fused, directly or via a suitable linker, to the second second binding member of said binding pair; and
said liposome or vesicle gives rise to a detectable signal and/or to a change in a detectable signal (and in particular, to a detectable signal that is generated by the first and second binding members of the binding pair and/or to a change in such a signal) when an agonist for the translayer protein that is present in the environment outside of the liposome or vesicle binds to the translayer protein.

Again, the invention also relates to method for providing such a liposome or vesicle which method comprises at least the step of incorporating said fusion proteins into a liposome or vesicle and/or of forming a liposome or vesicle in the presence of said fusion proteins.

Such liposomes or vesicles that comprise such first and second fusion proteins can be as further described herein, and are preferably such that they have the translayer protein suitably anchored or otherwise incorporated into the wall or membrane of the liposome or vesicle and spans said wall or membrane, more preferably such that at least one part of the amino acid sequence of the translayer protein extends out (as defined herein) into the environment outside of the liposome or vesicle and at least one other part of the amino acid sequence of the translayer protein extends out (as defined herein) into the environment inside the liposome or vesicle.

Said liposomes or vesicles are also preferably such that the first and second binding members of the binding pair can come into contact or in close proximity to each other when the second fusion protein binds (directly or indirectly, as described herein) to the translayer protein that forms part of the first fusion protein. It will be clear to the skilled person that this generally means that the first and second binding members of the binding pair will be present in (as defined herein) the same environment relative to the wall or membrane of the liposome or vesicle. Preferably, the liposomes or vesicles are such that the first and second binding members of the binding pair will both be present in (as defined herein) the environment inside the liposome or vesicle.

Again, in the aspects of the invention that relate to liposomes or vesicles that contain such a first and second fusion protein, the translayer protein, the protein that can bind directly or indirectly to the translayer protein, the members of the binding pair and any linkers used can all be as further described herein.

In further aspects, the invention also relates to methods, and in particular assay methods or screening methods, that involve the use of the liposomes or vesicles described herein. As further described herein, such assay and screening methods can in particular be used to identify compounds and other chemical entities that bind to (and in particular specifically bind to) the translayer protein, that can modulate the translayer protein and/or that modulate the signaling, signaling pathway and/or biological or physiological activitie(s) in which the translayer protein, its signaling and/or its signaling pathway is involved. As such, the liposomes or vesicles described herein can be used in methods to identify compounds or other chemical entities that can act as an agonist, antagonist, inverse agonist, inhibitor or modulator (such as an allosteric) modulator of the translayer protein.

The invention also relates to uses of the liposomes or vesicles described herein, in particular in assay and screening methods and techniques. Such methods and uses can again be as further described herein for methods and uses of the arrangements of the invention.

Again, in all these aspects, such liposomes or vesicles and uses thereof are preferably as further described herein.

It will be clear to the skilled person that the compounds which are discovered, developed, generated and/or optimized using the methods and techniques which are described herein may be used for any suitable or desired purpose. Said purpose will generally be associated with the target against which the compounds have been screened/generated, with the signaling, pathway(s) and/or mechanism of action with which the target is associated, and/or with the biological, physiological and/or pharmacological functions in which said target, pathway(s), signaling and/or mechanism of action are involved. Usually, and preferably, a compound of the invention will be such, and/or will be chosen such, that it is capable of modulating said target, signaling, pathway(s), mechanism of action and/or said biological, physiological and/or pharmacological functions in a desired or intended manner. As mentioned herein, this modulation can take any desired or intended form, including but not limited to upregulation and downregulation of the target, signaling, pathway(s), mechanism of action and/or said biological, physiological and/or pharmacological functions. As such, the compounds of the invention can for example function as agonists, antagonists, inverse agonists, inhibitors or another type of modulator (such as an allosteric modulator) for said target and/or its signaling, pathway(s), mechanism of action and/or said biological, physiological and/or pharmacological functions. All of this can be determined using suitable in vitro, cellular and/or in vivo assays (such as suitable efficacy or potency assays) and/or suitable animal models, depending on the specific target, signaling, pathway(s), mechanism of action and/or said biological, physiological and/or pharmacological functions involved. Suitable assays and models will be clear to the skilled person.

Usually, when a compound of the invention is an agonist (or antagonist, respectively) of the target, it will also be an agonist (or antagonist, respectively) of the signaling, pathway(s), mechanism of action and/or said biological, physiological and/or pharmacological functions in which the target is involved. However, as will be clear to the skilled person, it is also possible (and not excluded from the scope of the invention) that a compound of the invention may, for example and without being limited to any kind of hypothesis or explanation, be an agonist (or antagonist, respectively) of the target or its signaling but that such action as an agonist (or antagonist, respectively) of the target or its signaling results in an action as an antagonist (or antagonist, respectively) with respect to the biological, physiological and/or pharmacological functions in which the target or signaling is involved.

In one aspect of the practice of the invention, the arrangements and methods described herein will be used to test whether a compound or ligand that is present in environment [A] (for example, in the extracellular environment if the invention is performed in cells or the environment outside of the liposome or vesicle if the invention is performed in a liposome or vesicle) is capable of generating a detectable signal when it is contacted with the arrangement of the invention (i.e. in a way that allows said compound or ligand to bind with to the binding site (8) on the translayer protein (2)). Similarly, when the methods and arrangements of the invention are used to screen a group, series or library of compounds or ligands, the methods and arrangements of the invention will be used to determine which compounds or ligands from said group, series or library generate a detectable signal (i.e. are “hits”).

Generally, in the invention, said detectable signal will be measured by measuring the signal that is (or can be) generated by the binding pair (6/7) (i.e. the signal that is generated when the first member (6) and the second member (7) come into contact with each other, come into close proximity to each other, or otherwise associate with each other to generate a detectable signal). It should be noted that, in the invention, usually a change in said signal is measured, and such change is also included within the term “generate a detectable signal” as used herein.

Said change can be either an increase in signal compared to a base level (which base level can also be below the detection limit of the equipment used to measure the signal, in which case there will be a signal detected in the presence of the compound of ligand where essentially no signal was measured before and this is also included within the term “increase in signal” as used herein) or a decrease in signal compared to a base level.

In the practice of the invention, when the translayer protein (2) is a GPCR or 7TM (and, as will be clear to the skilled person based in the disclosure herein, usually also when the translayer protein (2) is another transmembrane protein involved in signal transduction), an increase in signal will indicate that the compound or ligand acts as an agonist of the receptor. Conversely, when the translayer protein (2) is a GPCR or 7TM (and usually also when the translayer protein (2) is another transmembrane protein involved in signal transduction) a decrease in signal will indicate that the compound or ligand acts as an inverse agonist of the receptor. Thus, with advantage, the methods and arrangements of the invention may make it possible to identify both agonists and inverse agonists of a GPCR or 7TM (or other receptor) and/or to distinguish agonists from inverse agonists (or visa versa). Reference is for example made to the results given in Example 6 and shown in FIG. 12.

It should be noted that the invention is not limited to any specific mechanism, explanation or hypothesis as to how the contact between the compound or ligand and (the binding site (8) on) the translayer protein (2) leads to a change in the detectable signal. However, it is assumed that one or more of the following mechanisms will be involved.

As mentioned herein, generally, the translayer protein (2) will be a protein that, without the presence of the compound or ligand, exists in an equilibrium between two or more conformations, and some of these conformations will have low(er) affinity for (or even essentially no affinity for) the binding interaction between the (binding site (9) on) the translayer protein (2) and the second ligand (4) compared to other conformations. Generally, in the invention, the level of detectable signal that is (or can be) measured at a certain point in time (or within a certain time interval) will depend on how much of the second ligand (4) (i.e. of the second fusion protein) binds or becomes bound to the translayer protein, as the binding of the second fusion protein to the translayer protein (2) will bring (more of) the second binding member (7) in proximity to the first binding member (6), thus leading to the detectable signal (or an increase in the detectable signal compared to the level of background signal that may be present due to binding of “free” second ligand to the binding member (6), which background level is usually insignificant or below the detection limit).

Thus, generally, in the invention, a shift in the conformational equilibrium of the translayer protein (2) from states with low(er) or essentially no affinity for the second ligand (4) towards states with binding affinity for the second ligand (4) and/or states with better binding affinity for said second ligand (4) will generally lead to an increase in the detectable signal.

It is assumed that in the invention, the contacting of the translayer protein (2) with a compound or ligand that acts as an agonist will either shift this equilibrium towards conformational states with binding affinity for the second ligand (4) and/or states with better binding affinity, thus leading to an increase in signal that can be detected. This can for example be because the presence of the agonistic compound or ligand allows for the formation of new conformational states (for example, the formation of complexes comprising the compound or ligand, the translayer protein and the second ligand) which cannot be formed when the compound or ligand is not present, because the agonistic compound or ligand stabilizes (or generally favors the formation of) conformational states that have high(er) affinity for the second ligand (4), and/or because the agonistic compound or ligand leads to new conformations that can bind the second ligand. Any one or more of these and other mechanisms (or any combination thereof) can be involved at any time, but the overall effect will be an increase of the amount of second ligand (4) that, at a certain moment in time (i.e. when the translayer protein (2) is in contact with the agonistic compound or ligand) and/or within a certain time interval (i.e. after the translayer protein (2) has been contacted with the agonistic compound or ligand), is associated with the translayer protein (2) and thus an increase in the amount of second binding member (7) that comes into contact or proximity to the first binding member (6) and thus to an increase in the detectable signal.

Based on the further description herein, it will also be clear to the skilled person that, because the translayer protein (2) exists in an equilibrium between states with no or low(er) affinity for the second ligand (2) and states with high(er) affinity for the second ligand (2), that even when the compound or ligand is not present, there will be a certain “basal” amount of the second fusion protein that is in contact with the second binding site (9) at any point in time of within a certain period of time. This basal level of binding will also lead to a certain basal level of detectable signal, which may be below the detection limit for the assay but in one specific aspect of the invention this basal signal is such that it is or can be detected (and/or the method of the invention is performed in such a way that it is detected). In such a case, an agonist will again lead to an increase in detectable signal compared to said basic level, but also an inverse agonist may shift the conformational equilibrium away from conformations with high(er) affinity for the second ligand (4) towards conformations with low(er) affinity for the second ligand (4). The result of this will be a decrease of the amount of second fusion protein that is bound to the translayer protein (2) at a certain moment in time and/or within a certain time interval, which will lead to a decrease in the detectable signal.

Thus, this aspect and set-up of the invention will make it possible to screen for inverse agonists and/or to test compounds and ligands for activity as an inverse agonist. With advantage, this aspect and set-up of the invention will also make it possible to screen or test for agonists and antagonists as part of the same run of the screening or assay.

Again, the invention is not limited to any specific mechanism, explanation or hypothesis as to how a compound or ligand acts as an inverse agonist for the translayer protein (2). However it is assumed that an inverse agonist may stabilize (or generally favor the formation of) conformational states that have low(er) affinity for the second ligand (4), may allow for the formation of new conformational states which cannot be formed when the compound or ligand is not present and which essentially cannot bind the second ligand (4) or only do so with low affinity, and/or may make it more difficult for the translayer protein to undergo a conformational change into states that have higher affinity for the second ligand (4) (for example, by increasing the activation energy required for the conformational change). Any one or more of these and other mechanisms (or any combination thereof) can be involved at any time, but the overall effect will be a decrease in the amount of second ligand (4) that, at a certain moment in time (i.e. when the translayer protein (2) is in contact with the inverse agonist) and/or within a certain time interval (i.e. after the translayer protein (2) has been contacted with the inverse agonist), is associated with the translayer protein (2) and thus a decrease in the amount of second binding member (7) that is into contact or proximity to the first binding member (6) (compared to the situation where the inverse agonist is not present) and thus to a decrease in the detectable signal (i.e. compared to a basal signal without the presence of the inverse agonist).

Generally, the methods of the invention will comprise providing an arrangement as described herein and then contacting said arrangement with the compound(s) or ligand(s) to be screened or tested, i.e. for a certain period of time (which will usually be chosen so as to achieve a suitable or desired assay or screening “window”, and which may be benchmarked against a suitable window set with one or more known agonists or inverse agonists of the receptor involved) and in one or more concentrations, for example, to set a dose response curve and/or to allow the determination of an IC50 or another desired parameter (again, these concentrations may be chosen based on experience obtained with one or more known agonists or inverse agonists of the receptor involved). This will generally be performed using techniques for assay validation known per se.

The methods of the invention can be performed in a suitable medium, which may be water, a buffer or another suitable aqueous medium. When the methods of the invention are performed using cells or vesicles, the medium is preferably suitably chosen so as to ensure or promote viability of the cells or stability of the vesicles used, respectively.

After the arrangement of the invention has brought into contact with the compound(s) or ligand(s) to be screened or tested, the level of the detectable signal is measured at one or more moments in time or continuously over a desired time interval. This can be performed in any manner known per se, mainly depending on the binding pair (6/7) that is being used. Suitable equipment will be clear to the skilled person and will for example include the equipment used in the Experimental Section below. The value(s) obtained may also be compared to reference values (for example to the value(s) obtained in the same assay with one or more known agonists or inverse agonists, the value(s) obtained for a blank or carrier, and/or reference values obtained from previous experiments).

Based on the further disclosure herein, the skilled person will be able to suitably select other conditions (such as temperature) and equipment for performing the methods of the invention. Reference is also made to the Experimental Part herein for some suitable but non-limiting conditions.

For screening purposes, in particular of libraries of compounds or ligands, the methods of the invention may be performed in a high-throughput screening (HTS) format. When the methods of the invention are to be performed using cells, suitable techniques for performing cellular assays in HTS format can be applied. Reference is for example made to the review article by Rajalingham, BioTechnologia, 97(3), 227-234 (2016) and to Zang et al., International Journal of Biotechnology for Wellness Industries, 2012, 1, 31-51.

In the preceding paragraphs, the invention has been described with reference to FIG. 1, which shows an embodiment of the invention in which the second ligand (4) has been selected to bind directly to the binding site (9) on the translayer protein (2). FIG. 2 shows an alternative embodiment of the invention, in which the second ligand (4) does not bind directly to the translayer protein (2), but binds to another protein which other protein in turn can bind to the binding site (9) on the translayer protein (2). In FIG. 2, said other protein (referred to herein for convenience as the “signaling protein”) is indicated as (5)—all other reference numbers in FIG. 2) are as defined herein for FIG. 1.

The overall principle of the embodiment shown in FIG. 2 is the same as for the method described herein for FIG. 1, in that the invention makes use of two fusion proteins that each comprise a member of the binding pair (6/7), and that binding of the first ligand (3) to the translayer protein (2) results in the first binding member (6) and the second binding member (7) of said binding pair coming into contact with, or in close proximity to, each other, giving rise to a detectable signal. Also, as with FIG. 1, and again without being limited to any specific mechanism, hypothesis or explanation, said signal will arise out of, increase or decrease as a result of, and/or otherwise be associated with a conformational change in the translayer protein (2) and/or a shift in the conformational equilibrium of the translayer protein (2), essentially as described with respect to FIG. 1. However, in the embodiment of FIG. 2, said conformational change or shift in the conformational equilibrium will not be caused by (or associated with) the binding of the second ligand (4) to the translayer protein, but instead by the binding of the signaling protein (5) to the translayer protein. The second ligand (4) will bind to the signaling protein (5) when bound to the translayer protein (2) and so give rise to the detectable signal.

In this embodiment, again without being limited to any specific mechanism, hypothesis or explanation, it may be that the signaling protein (5) will only bind to those conformations of the translayer protein (2) that are associated with the binding of the first ligand (3) to the translayer protein (2), so that the first and second binding member of the binding pair (6/7) can only come into contact or close proximity when the signaling protein (5) is bound to the translayer protein (2). It is also possible that the signaling protein (5) itself undergoes a conformational change upon binding to the translayer protein (2) and that the second ligand (4) is selected such that it essentially only binds (or binds with higher affinity) to the conformation of the signaling protein (5) that arises upon binding to the translayer protein (2). It is also possible that the signaling protein (5), upon binding to the translayer protein (2), forms a complex with (or otherwise becomes associated with) other proteins, and that the second ligand (4) binds (or binds with higher affinity) to the complex.

### EXPERIMENTAL PART

In the arrangements of the invention that are illustrated by Examples 1 to 3 below, a second fusion protein is used that binds indirectly to the relevant receptor. In said examples, the second fusion protein comprises either a VHH domain that binds to a G-protein complex (CA4435) or a VHH domain that binds to G-protein (CA4427).

In the arrangements of the invention that are illustrated by Examples 4 to 12 below, a second fusion protein is used that binds directly to the relevant receptor. In said examples, each of the second fusion proteins used comprises a VHH domain that binds to the G-protein binding site on the receptor used.

Table 1 below give the amino acid sequences of some of the fusion proteins, ConfoBodies and other elements referred to in the Examples below.

TABLE 1
amino acid sequences

| SEQ ID | | |
|---|---|---|
| NO: | Description | Amino acid sequence |
| 1 | CA4435 | QVQLQESGGGLVQPGGSLRLSCAASGFTFSNYKMNWVRQAPGKGLEWVSDISQSG |
| | | ASISYTGSVKGRFTISRDNAKNTLYLQMNSLKPEDTAVYYCARCPAPFTRDCFDVT |
| | | STTYAYRGQGTQVTVSS |
| 2 | CA4437 | QVQLQESGGGFVQAGGSLRLSCAASGSIFSKNTMAWFRQAPGKERELVAASPTGG |
| | | STAYKDSVKGRFTISRDSAKNTVLLQMNVLKPEDTAVYYCHLRQNNRGSWFHYW |
| | | GQGTQVTVSS |
| 3 | hemagglutinin | MKTIIALSYIFCLVFA |
| | (HA) protein | |
| | signal peptide | |
| 4 | FLAG-tag | DYKDDDDA |
| 5 | Linker | GAQGNS-GSSGGGGSGGGGSSG |
| 6 | Linker | GSSGGGGSGGGGSSG |
| 7 | large subunit | VFTLEDFVGDWEQTAAYNLDQVLEQGGVSSLLQNLAVSVTPIQRIVRSGENALKID |
| | of the | IHVIIPYEGLSADQMAQIEEVFKVVYPVDDHHFKVILPYGTLVIDGVTPNMLNYFGR |
| | NanoLuc | PYEGIAVFDGKKITVTGTLWNGNKIIDERLITPDGSMLFRVTINS |
| | luciferase | |
| 8 | small subunit | VTGYRLFEEIL |
| | of the | |
| | NanoLuc | |
| | luciferase | |
| 9 | MC4R fusion | MKTIIALSYIFCLVFADYKDDDDKAVNSTHRGMHTSLHLWNRSSYRLHSNASESLG |
| | | KGYSDGGCYEQLFVSPEVFVTLGVISLLENILVIVAIAKNKNLHSPMYFFICSLAVA |
| | | DMLVSVSNGSETIVITLLNSTDTDAQSFTVNIDNVIDSVICSSLLASICSLLSIAVDRY |
| | | FTIFYALQYHNIMTVKRVGIIISCIWAACTVSGILFITYSDSSAVIICLITMFFTMLALM |
| | | ASLYVHMFLMARLHIKRIAVLPGTGAIRQGANMKGAITLTILIGVFVVCWAPFFLH |
| | | LIFYISCPQNPYCVCFMSHFNLYLILIMCNSIIDPLIYALRSQELRKTFKEIICCYPLGG |
| | | LCDLSSRYGAQGNSGSSGGGGSGGGGSSGVFTLEDFVGDWEQTAAYNLDQVLEQ |
| | | GGVSSLLQNLAVSVTPIQRIVRSGENALKIDIHVIIPYEGLSADQMAQIEEVFKVVYP |
| | | VDDHHFKVILPYGTLVIDGVTPNMLNYFGRPYEGIAVFDGKKITVTGTLWNGNKII |
| | | DERLITPDGSMLFRVTINS |
| 10 | GLP-1R | MKTIIALSYIFCLVFADYKDDDDAMAGAPGPLRLALLLLGMVGRAGPRPQGATVS |
| | fusion | LWETVQKWREYRRQCQRSLTEDPPPATDLFCNRTFDEYACWPDGEPGSFVNVSCP |
| | | WYLPWASSVPQGHVYRFCTAEGLWLQKDNSSLPWRDLSECEESKRGERSSPEEQL |
| | | LFLYIIYTVGYALSFSALVIASAILLGFRHLHCTRNYIHLNLFASFILRALSVFIKDAA |
| | | LKWMYSTAAQQHQWDGLLSYQDSLSCRLVFLLMQYCVAANYYWLLVEGVYLYT |
| | | LLAFSVLSEQWIFRLYVSIGWGVPLLFVVPWGIVKYLYEDEGCWTRNSNMNYWLII |
| | | RLPILFAIGVNFLIFVRVICIVVSKLKANLMCKTDIKCRLAKSTLTLIPLLGTHEVIFAF |
| | | VMDEHARGTLRFIKLFTELSFTSFQGLMVAILYCFVNNEVQLEFRKSWERWRLEHL |
| | | HIQRDSSMKPLKCPTSSLSSGATAGSSMYTATCQASCSGAQGNSGSSGGGGSGGGG |
| | | SSGVFTLEDFVGDWEQTAAYNLDQVLEQGGVSSLLQNLAVSVTPIQRIVRSGENAL |
| | | KIDIHVIIPYEGLSADQMAQIEEVFKVVYPVDDHHFKVILPYGTLVIDGVTPNMLNY |
| | | FGRPYEGIAVFDGKKITVTGTLWNGNKIIDERLITPDGSMLFRVTINS |
| 11 | Beta-2AR | MKTIIALSYIFCLVFADYKDDDDAENLYFQGFGQPGNGSAFLLAPNRSHAPDHDVT |
| | fusion | QQRDEVWVVGMGIVMSLIVLAIVFGNVLVITAIAKFERLQTVTNYFITSLACADLV |
| | | MGLAVVPFGAAHILMKMWTFGNFWCEFWTSIDVLCVTASIETLCVIAVDRYFAITS |
| | | PFKYQSLLTKNKARVIILMVWIVSGLTSFLPIQMHWYRATHQEAINCYAEETCCDFF |
| | | TNQAYAIASSIVSFYVPLVIMVFVYSRVFQEAKRQLQKIDKSEGRFHVQNLSQVEQ |
| | | DGRTGHGLRRSSKFCLKEHKALKTLGIIMGTFTLCWLPFFIVNIVHVIQDNLIRKEV |
| | | YILLNWIGYVNSGFNPLIYCRSPDFRIAFQELLCLRRSSLKAYGNGYSSNGNTGEQS |
| | | GGAQGNSGSSGGGGSGGGGSSGVFTLEDFVGDWEQTAAYNLDQVLEQGGVSSLL |
| | | QNLAVSVTPIQRIVRSGENALKIDIHVIIPYEGLSADQMAQIEEVFKVVYPVDDHHF |
| | | KVILPYGTLVIDGVTPNMLNYFGRPYEGIAVFDGKKITVTGTLWNGNKIIDERLITP |
| | | DGSMLFRVTINS |
| 12 | MOR | MKTIIALSYIFCLVFADYKDDDDAMAPTNASNCTDALAYSSCSPAPSPGSWVNLSH |
| | fusion | LDGNLSDPCGPNRTDLGGRDSLCPPTGSPSMITAITIMALYSIVCVVGLFGNFLVMY |
| | | VIVRYTKMKTATNIYIFNLALADALATSTLPFQSVNYLMGTWPFGTILCKIVISIDYY |
| | | NMFTSIFTLCTMSVDRYIAVCHPVKALDFRTPRNAKIINVCNWILSSAIGLPVMFMA |
| | | TTKYRQGSIDCTLTFSHPTWYWENLLKICVFIFAFIMPVLIITVCYGLMILRLKSVRM |
| | | LSGSKEKDRNLRRITRMVLVVVAVFIVCWTPIHIYVIIKALVTIPETTFQTVSWHFCI |
| | | ALGYTNSCLNPVLYAFLDENFKRCFREFCIPTSSNIGAQGNSGSSGGGGSGGGGSSG |
| | | VFTLEDFVGDWEQTAAYNLDQVLEQGGVSSLLQNLAVSVTPIQRIVRSGENALKID |
| | | IHVIIPYEGLSADQMAQIEEVFKVVYPVDDHHFKVILPYGTLVIDGVTPNMLNYFGR |
| | | PYEGIAVFDGKKITVTGTLWNGNKIIDERLITPDGSMLFRVTINS |
| 13 | M2 fusion | MKTIIALSYIFCLVFADYKDDDDAMNNSTNSSNNSLALTSPYKTFEVVFIVLVAGSL |
| | | SLVTIIGNILVMVSIKVNRHLQTVNNYFLFSLACADLIIGVFSMNLYTLYTVIGYWPL |
| | | GPVVCDLWLALDYVVSNASVMNLLIISFDRYFCVTKPLTYPVKRTTKMAGMMIAA |
| | | AWVLSFILWAPAILFWQFIVGVRTVEDGECYIQFFSNAAVTFGTAIAAFYLPVIIMT |
| | | VLYWHISRASKSRIKKDKKEPVANQDPVSKKPPPSREKKVTRTILAILLAFIITWAPY |
| | | NVMVLINTFCAPCIPNTVWTIGYWLCYINSTINPACYALCNATFKKTFKHLLMGAQ |
| | | GNSGSSGGGGSGGGGSSGVFTLEDFVGDWEQTAAYNLDQVLEQGGVSSLLQNLA |
| | | VSVTPIQRIVRSGENALKIDIHVIIPYEGLSADQMAQIEEVFKVVYPVDDHHFKVILP |
| | | YGTLVIDGVTPNMLNYFGRPYEGIAVFDGKKITVTGTLWNGNKIIDERLITPDGSML |
| | | FRVTINS |
| 14 | AT1R | MKTIIALSYIFCLVFADYKDDDDAMILNSSTEDGIKRIQDDCPKAGRHNYIFVMIPTL |
| | fusion | YSIIFVVGIFGNSLVVIVIYFYMKLKTVASVFLLNLALADLCFLLTLPLWAVYTAME |
| | | YRWPFGNYLCKIASASVSFNLYASVFLLTCLSIDRYLAIVHPMKSRLRRTMLVAKV |
| | | TCIIIWLLAGLASLPAIIHRNVFFIENTNITVCAFHYESQNSTLPIGLGLTKNILGFLFPF |
| | | LIILTSYTLIWKALKKAYEIQKNKPRNDDIFKIIMAIVLFFFFSWIPHQIFTFLDVLIQL |
| | | GIIRDCRIADIVDTAMPITICIAYFNNCLNPLFYGFLGKKFKRYFLQLLKYGAQGNSG |
| | | SSGGGGSGGGGSSGVFTLEDFVGDWEQTAAYNLDQVLEQGGVSSLLQNLAVSVTP |
| | | IQRIVRSGENALKIDIHVIIPYEGLSADQMAQIEEVFKVVYPVDDHHFKVILPYGTLV |
| | | IDGVTPNMLNYFGRPYEGIAVFDGKKITVTGTLWNGNKIIDERLITPDGSMLFRVTI |
| | | NS |
| 15 | XA8633 | MQVQLQESGGGLVRPGGSRRLSCVDSERTSYPMGWFRRAPGKEREFVASITWSGI |
| | fusion | DPTYADSVADRFTISRDVANNTLYLQMNSLKHEDTAVYYCAARAPVGQSSSPYDY |
| | | DYWGQGTQVTVSSGSSGGGGSGGGGSSGVTGYRLFEEIL |
| 16 | CA2780 | MQVQLQESGGGLVQAGGSLRLSCAASGSIFSINTMGWYRQAPGKQRELVAAIHSG |
| | fusion | GSTNYANSVKGRFTISRDNAANTVYLQMNSLKPEDTAVYYCNVKDYGAVLYEYD |
| | | YWGQGTQVTVSSGSSGGGGSGGGGSSGVTGYRLFEEIL |
| 17 | Nb9-8 | MQVQLVESGGGLVQAGDSLRLSCAASGFDFDNFDDYAIGWFRQAPGQEREGVSCI |
| | fusion | DPSDGSTIYADSAKGRFTISSDNAENTVYLQMNSLKPEDTAVYVCSAWTLFHSDEY |
| | | WGQGTQVTVSSGSSGGGGSGGGGSSGVTGYRLFEEIL |
| 18 | NbAT110i1 | MQVQLQESGGGLVQAGGSLRLSCAASGNIFDVDIMGWYRQAPGKERELVASITDG |
| | fusion | GSTDYADSVKGRFTISRDNAKNTVYLQMNSLKPEDTAVYYCAAVAYPDIPTYFDY |
| | | DSDNFYWGQGTQVTVSSGSSGGGGSGGGGSSGVTGYRLFEEIL |
| 19 | CA4437 | MQVQLQESGGGFVQAGGSLRLSCAASGSIFSKNTMAWFRQAPGKERELVAASPTG |
| | fusion | GSTAYKDSVKGRFTISRDSAKNTVLLQMNVLKPEDTAVYYCHLRQNNRGSWFHY |
| | | WGQGTQVTVSSGSSGGGGSGGGGSSGVTGYRLFEEIL |
| 20 | CA4435 | MQVQLQESGGGLVQPGGSLRLSCAASGFTFSNYKMNWVRQAPGKGLEWVSDISQ |
| | fusion | SGASISYTGSVKGRFTISRDNAKNTLYLQMNSLKPEDTAVYYCARCPAPFTRDCFD |
| | | VTSTTYAYRGQGTQVTVSSGSSGGGGSGGGGSSGVTGYRLFEEIL |
| 21 | NbAT110i1 | MQVQLQESGGGLVQAGGSLRLSCAASGNIFDVDIMGWYRQAPGKERELVASITDG |
| | | GSTDYADSVKGRFTISRDNAKNTVYLQMNSLKPEDTAVYYCAAVAYPDIPTYFDY |
| | | DSDNFYWGQGTQVTVSS |
| 22 | APL | MKTIIALSYIFCLVFADYKDDDDAMEEGGDFDNYYGADNQSECEYTDWKSSGALI |
| | receptor- | PAIYMLVFLLGTTGNGLVLWTIVRYTKMKRRSADIFIASLAVADLTFVVTLPLWAT |
| | MOR chimer | YTYRDYDWPFGTFFCKLSSYLIFVNMYASVFCLTGLSFDRYLAICHPVKALDFRTP |
| | | RNGAVATAVLWVLAALLAMPVMVLRTTGDLENTTKVQCYMDYSMVATVSSEW |
| | | AWEVGLGVSSTTVGFVVPFTIMLTCYGLMILRLKSVRMLSGSKEKDRNLRRILSIIV |
| | | VLVVTFALCWMPYHLVKTLYMLGSLLHWPCDFDLFLMNIFPYCTCISYVNSCLNPF |
| | | LYAFLDENFKRCFREFCIPTSSNI |
| 23 | OX2-MOR | MSGTKLEDSPPCRNWSSASELNETQEPFLNPTDYDDEEFLRYLWREYLHPKEYEW |
| | chimer | VLIAGYIIVFVVALIGNVLVMYVIVRYTKMKTATNYFIVNLSLADVLVTITCLPATL |
| | | VVDITETWFFGQSLCKVIPYLQTVSVSVSVLTLSCIALDRYIAVCHPVKALDFRTPR |
| | | NARNSIVIIWIVSCIIMIPQAIVMECSTVFPGLANKTTLFTVCDERWGGETYPKMYHI |
| | | CFFLVTYMAPLCLMVLAYGLMILRLKSVRMLSGSKEKDRARRKTARMLMIVLLVF |
| | | AICYLPISILNVLKRVFGMFAHTEDRETVYAWFTFSHWLVYANSAANPIIYAFLDEN |
| | | FKRCFREFCIPTSSNI |
| 24 | XA8633 | QVQLVESGGGLVRPGGSRRLSCVDSERTSYPMGWFRRAPGKEREFVASITWSGIDP |
| | | TYADSVADRFTISRDVANNTLYLQMNSLKHEDTAVYYCAARAPVGQSSSPYDYDY |
| | | WGQGTQVTVSS |
| 25 | Nb 9-8 | QVQLQESGGGLVQAGDSLRLSCAASGFDFDNFDDYAIGWFRQAPGQEREGVSCID |
| | | PSDGSTIYADSAKGRFTISSDNAENTVYLQMNSLKPEDTAVYVCSAWTLFHSDEY |
| | | WGQGTQVTVSS |
| 26 | CA2780 | QVQLQESGGGLVQAGGSLRLSCAASGSIFSINTMGWYRQAPGKQRELVAAIHSGGS |
| | | TNYANSVKGRFTISRDNAANTVYLQMNSLKPEDTAVYYCNVKDYGAVLYEYDY |
| | | WGQGTQVTVSS |

### Example 1: Screening Assay for Melanocortin 4 Receptor

A melanocortin 4 receptor screening assay is performed with Human Embryonic Kidney (HEK) 293T cells transiently transfected with a pBiT1.1C (Promega) expression vector encoding human full length melanocortin 4 receptor (MC4R) and with a pcDNA3.1 expression vector encoding CA4437. The MC4R expression vector has a cleavable derived from influenza virus (MKTIIALSYIFCLVFA; SEQ ID NO:3) followed by a FLAG-tag sequence (DYKDDDDA; SEQ ID NO:4) and is fused on the C-terminus via a flexible linker (GAQGNS-GSSGGGGSGGGGSSG; SEQ ID NO:5) to the large subunit of the NanoLuc luciferase (LgBit; SEQ ID NO:7). CA4437 (SEQ ID NO:2) is fused via a flexible linker (GSSGGGGSGGGGSSG; SEQ ID NO:6) on the C-terminus to the small subunit of the NanoLuc luciferase (SmBiT; SEQ ID NO:8).

HEK 293T cells are seeded in 6-well plate at 1 million cells per well and allowed to attach for at least 16 hours prior to transfection. HEK 293T cells are maintained at 37° C., 5% CO2, under humidified atmosphere in Dulbecco's Modified Eagle's Medium (DMEM) supplemented with 10% heat-inactivated FBS, 100U/ml penicillin, 100 μg/ml streptomycin, 4 mM L-Glutamine and 1 mM sodium pyruvate (Gibco). MC4R-LgBiT and CA4437-SmBiT are transfected using a 1:1 DNA ratio (corresponds to 1.5 μg for each construct) and X-tremeGENE HP DNA transfection reagent (Roche) was used for transfection using a 3 to 1 ratio of microliter transfection reagent volume to microgram DNA.

24 hours after transfection, the cells are harvested using culture medium and washed twice with Opti-MEM I reduced serum medium without phenol red (Gibco) to remove any remaining FBS. Transfected cells are seeded in white 96-well flat bottom tissue-culture treated plate (Corning; 3917) using a density of 50,000 cells per well (90 μl). After a 30 minutes incubation time at 37° C., 5% CO2, 20 μl of compound solution prepared as 5.5× stock solution in Opti-MEM is added to each well, gently mixed by hand and incubated for 1 hour at room temperature. Solvent controls were run in all experiments. Agonists NDP-alpha-MSH (Tocris, 3013) is applied at different concentrations in the assay. The Nano-Glo® Live Cell Substrate (Promega) is diluted 20× in the Nano-Glo® LCS dilution buffer to make a 5× stock for addition to the cell culture medium. 25 μl of diluted Nano-Glo® substrate is added to each well, gently mixed by hand and luminescence is continuously monitored for 120 minutes (one measurement every 2 minutes) on Envision or SpectraMax i3x plate reader.

Curve fitting and statistical analysis is performed in GraphPad Prism and data are represented as mean Area Under the Curve (AUC) and standard error on the mean. 2-3 replicates are implemented per data point. Data is represented as normalized AUC which corresponds to the ratio AUC(sample) over AUC(blank).

The sequence of the MC4R fusion used in this Example is given in Table 1 as SEQ ID NO:9 (in the final construct, the last amino acid of the FLAG-tag was K instead of A). The sequence of the CA4437 fusion used in this Example is given in Table 1 as SEQ ID NO:19, and the sequence of the CA4435 fusion used in this Example is given in Table 1 as SEQ ID NO:20.

The results are shown in FIG. 4. As can be seen, using the assay described in this Example, it was possible to establish a dose response curve for NDP-alpha-MSH.

### Example 2: Screening Assay for Receptor of GLP-1

Same process as in Example 1 was used, except for that first Nano-Glo was added to the seeded cells, followed by compound addition. 50,000 cells per well are seeded in white 96-well flat bottom tissue-culture treated plate and incubated for 80 minutes at 37° C., 5% CO2 before addition of Nano-Glo. The Nano-Glo® Live Cell Substrate (Promega) is diluted 20× in the Nano-Glo® LCS dilution buffer to make a 5× stock for addition to the cell culture medium. 25 μl of diluted Nano-Glo® substrate is added to each well, gently mixed by hand and luminescence is continuously monitored on Envision plate reader until stabilization of the signal (40 minutes for this assay). Next, 20 μl of agonist solution prepared as 6.75× stock solution in Opti-MEM is added to each well, gently mixed by hand and luminescence is continuously monitored for 120 minutes (one measurement every 2 minutes) at room temperature on Envision plate reader.

A glucagon-like peptide 1 receptor screening assay is performed with Human Embryonic Kidney (HEK) 293T cells transiently transfected with a pBiT1.1C (Promega) expression vector encoding human full length (residues 1-463) Glucagon-like peptide 1 receptor (GLP-1R) and with a pcDNA3.1 expression vector encoding CA4437 or CA4435 (VHH). The GLP-1 receptor expression vector has a cleavable hemagglutinin (HA) protein signal peptide derived from influenza virus (MKTIIALSYIFCLVFA; SEQ ID NO:3) followed by a FLAG-tag sequence (DYKDDDDA; SEQ ID NO:4) and is fused on the C-terminus via a flexible linker (GAQGNS-GSSGGGGSGGGGSSG; SEQ ID NO:5) to the large subunit of the NanoLuc luciferase (LgBit; SEQ ID NO:7). CA4437 (SEQ ID NO:2) and CA4435 (SEQ ID NO:1) are fused via a flexible linker (GSSGGGGSGGGGSSG; SEQ ID NO:6) on the C-terminus to the small subunit of the NanoLuc luciferase (SmBiT; SEQ ID NO:8). The ratio of DNA of the GLP-1R expressing pBiT1.1C vector and CA4437 expressing pcDNA3.1 vector during transfection was 2:1 (corresponds to 0.5 μg of GLP-1R-LgBiT and 0.25 μg of CA4437-SmBiT). The ratio of DNA of the GLP-1R expressing pBiT1.1C vector and CA4435 expressing pcDNA3.1 vector during transfection was 1:1 (corresponds to 1.5 μg of each construct).

Agonists GLP-1 (7-36) amide (Tocris, 2082), Glucagon (ChemScene, CS-5936), Oxyntomodulin (Tocris, 2094), Exendin-4 (ChemScene, CS-1174), Liraglutide (ChemScene, CS-4545), Taspoglutide (ChemScene, CS-6174), Lixisenatide (ChemScene, CS-5788), Albiglutide (Abcam, ab231357), Semaglutide (ChemScene, CS-0080402), GLP-1R agonist-1 (ChemScene, CS-0062504, extracted from patent: WO 2018/109607 A1) are applied at different concentrations in the assay.

Curve fitting and statistical analysis is performed in GraphPad Prism and data are represented as mean Area Under the Curve (AUC) and standard error on the mean. 2-3 replicates are implemented per data point. Data is represented as normalized AUC which corresponds to the ratio AUC (sample) over AUC (blank) and is normalized for potential well-to-well variations due to seeding.

The sequence of the GLP-1R fusion used in this Example is given in Table 1 as SEQ ID NO:10. The sequence of the CA4437 fusion used in this Example is given in Table 1 as SEQ ID NO:19, and the sequence of the CA4435 fusion used in this Example is given in Table 1 as SEQ ID NO:20.

The results are shown in FIGS. 5A to 5C (for the assay using CA4437) and FIG. 6 (for the assay using CA4435). As can be seen, using each of these assays, it was possible to establish a dose response curve for the indicated compounds.

### Example 3: Screening Assay for Beta-2 Adrenergic Receptor

Same process as in Example 2 was used, except for that seeded cells were incubated for 1 hour at 37° C., 5% CO2 before addition of Nano-Glo. pBiT1.1-C expression vector encoding human truncated (residues 2-365) Beta-2 adrenergic β2AR) vector has a cleavable hemagglutinin (HA) protein signal peptide derived from influenza virus (MKTIIALSYIFCLVFA; SEQ ID NO:3) followed by a FLAG-tag sequence (DYKDDDDA; SEQ ID NO:4) and is fused on the C-terminus via a flexible linker (GAQGNS-GSSGGGGSGGGGSSG; SEQ ID NO:5) to the large subunit of the NanoLuc luciferase (LgBit; SEQ ID NO:7). CA4435 (SEQ ID NO:1) and CA4437 (SEQ ID NO:2) are fused via a flexible linker (GSSGGGGSGGGGSSG; SEQ ID NO:6) on the C-terminus to the small subunit of the NanoLuc luciferase (SmBiT; SEQ ID NO:8). The ratio of DNA of the (32AR expressing pBiT1.1C vector and CA4435 expressing pcDNA3.1 vector during transfection was 1:2 (corresponds to 0.5 μg of (32AR expressing vector and 1 μg of CA4435 expressing vector). The ratio of DNA of the (32AR expressing pBiT1.1C vector and CA4437 expressing pcDNA3.1 vector during transfection was 1:2 (corresponds to 0.5 μg of (32AR expressing vector and 1 μg of CA4437 expressing vector). Agonists isoprotenerol (Sigma, 15627), pindolol (Sigma, P0778) and inverse agonist ICI 118,551 (Sigma, 1127) are applied at a single concentration in the assay. Samples and vehicle are prepared in Opti-MEM I reduced medium containing final 1% DMSO.

The sequence of the Beta-2AR fusion used in this Example is given in Table 1 as SEQ ID NO:11. The sequence of the CA4437 fusion used in this Example is given in Table 1 as SEQ ID NO:19, and the sequence of the CA4435 fusion used in this Example is given in Table 1 as SEQ ID NO:20.

The results are shown in FIGS. 7A and 7B (for the assay using CA4437) and FIGS. 8A and 8B (for the assay using CA4435). As can be seen, each assay was able to distinguish the agonists from the reference (blank) and the inverse agonist.

In a further experiment, the use of a bivalent construct comprising both CA4435 and CA4437 (linked by a 35GS linker, i.e. as CA4435-35GS-CA4437) was compared in this assay set up with the use of CA44335 alone and CA4437 alone. This bivalent construct is biparatopic for the G-protein. The results are shown in FIGS. 8C (CA4437 alone), 8D (CA4435 alone) and 8E (CA4435-35GS-CA4437). The compounds used in this comparative experiment are (bars from left to right): blank, isoproterenol at 10 μM, isoproterenol at 1 μM, isoproterenol at 100 nM, a first compound (fragment) that has been separately identified as an agonist of (32AR at 100 μM, a second compound (fragment) that has been separately identified as an agonist of (32AR at 200 μM, a third compound (fragment) that has been separately identified as an agonist of (32AR at 200 μM, pindolol at 10 μM, salbutamol at 10 μM, ICI-118,561 at 10 μM, and carazolol at 10 μM.

Again, both the assays with the monovalent ISVDs and the assay with the bivalent/biparatopic ISVD fusion was able to distinguish the agonists from the reference (blank) and the inverse agonist ICI-118,561. Also, as can be seen from the results shown in FIGS. 8C to 8E, the use of the bivalent construct resulted in an improvement in sensitivity of the assay. These findings were separately confirmed in an experiment involving an assay for the receptor of GLP-1, similar to the set-up described in Example 2 (data not shown).

### Example 4: Screening Assay for μOpioid Receptor

The μopioid receptor screening assay is performed with Human Embryonic Kidney (HEK) 293T cells transiently transfected with a pBiT1.1C (Promega) expression vector encoding human truncated (residues 6-360) μopioid receptor (MOR) and with a pcDNA3.1 expression vector encoding XA8633 (VHH, SEQ ID NO:19 in WO14/118297 and SEQ ID NO:24 herein). The μopioid receptor expression vector has a cleavable hemagglutinin (HA) protein signal peptide derived from influenza virus (MKTIIALSYIFCLVFA; SEQ ID NO:3) followed by a FLAG-tag sequence (DYKDDDDA; SEQ ID NO:4) and is fused on the C-terminus via a flexible linker (GAQGNS-GSSGGGGSGGGGSSG; SEQ ID NO:5) to the large subunit of the NanoLuc luciferase (LgBit; SEQ ID NO:7). XA8633 is fused via a flexible linker (GSSGGGGSGGGGSSG; SEQ ID NO:6) on the C-terminus to the small subunit of the NanoLuc luciferase (SmBiT; SEQ ID NO:8).

HEK 293T cells are seeded in 6-well plate at 1 million cells per well and allowed to attach for at least 16 hours prior to transfection. HEK 293T cells are maintained at 37° C., 5% CO2, under humidified atmosphere in Dulbecco's Modified Eagle's Medium (DMEM) supplemented with 10% heat-inactivated FBS, 100U/ml penicillin, 100 μg/ml streptomycin, 4 mM L-Glutamine and 1 mM sodium pyruvate (Gibco). MOR-LgBiT and XA8633-SmBiT are transfected using a 1:1 DNA ratio (corresponds to 1.5 μg for each construct) and X-tremeGENE HP DNA transfection reagent (Roche) was used for transfection using a 3 to 1 ratio of microliter transfection reagent volume to microgram DNA.

24 hours after transfection, the cells are harvested using culture medium and washed twice with Opti-MEM I reduced serum medium without phenol red (Gibco) to remove any remaining FBS. Transfected cells are seeded in white 96-well flat bottom tissue-culture treated plate (Corning; 3917) using a density of 50,000 cells per well (90 μl). After a 30 minutes incubation time at 37° C., 5% CO2, 20 μl of compound solution (agonist or antagonist) prepared as 5.5× stock solution in Opti-MEM is added to each well, gently mixed by hand and incubated for 1 hour at room temperature. Solvent controls were run in all experiments. Agonists DAMGO (Tocris, 1171), PZM21 (Medchemexpress, HY-101386), TRV130 (Advanced ChemBlocks, M15340), hydromorphone (Sigma Aldrich, H5136) and antagonist naloxone (Tocris, 599) are applied at different concentrations in the assay. The Nano-Glo® Live Cell Substrate (Promega) is diluted 20× in the Nano-Glo® LCS dilution buffer to make a 5× stock for addition to the cell culture medium. 25 μl of diluted Nano-Glo® substrate is added to each well, gently mixed by hand and luminescence is continuously monitored for 120 minutes (one measurement every 2 minutes) on Envision or SpectraMax i3x plate reader.

Curve fitting and statistical analysis is performed in GraphPad Prism and data are represented as mean Area Under the Curve (AUC) and standard error on the mean. 2-3 replicates are implemented per data point. Data is represented as normalized AUC which corresponds to the ratio AUC(sample) over AUC(blank).

The sequence of the MOR fusion used in this Example is given in Table 1 as SEQ ID NO:12. The sequence of the XA8633 fusion used in this Example is given in Table 1 as SEQ ID NO:15.

The results are shown in FIGS. 9 and 10. As can be seen, using the assay of this example, it was possible to distinguish the agonists from the antagonists and to establish a dose response curve for the agonists.

### Example 5: Screening Assay for Muscarinic Acetylcholine Receptor M2

Same process as in Example 4 was used, except for that M2 was expressed in the vector pcDNA3.1 instead of the vector pBiT1.1C and except for that first Nano-Glo was added to the seeded cells, followed by compound addition. 50,000 cells per well are seeded in white 96-well flat bottom tissue-culture treated plate and incubated for 1 hour at 37° C., 5% CO2 before addition of Nano-Glo. The Nano-Glo® Live Cell Substrate (Promega) is diluted 20× in the Nano-Glo® LCS dilution buffer to make a 5× stock for addition to the cell culture medium. 25 μl of diluted Nano-Glo® substrate is added to each well, gently mixed by hand and luminescence is continuously monitored on Envision plate reader until stabilization of the signal. Next, 20 μl of agonist solution prepared as 6.75× stock solution in Opti-MEM is added to each well, gently mixed by hand and luminescence is continuously monitored for 120 minutes (one measurement every 2 minutes) at room temperature on Envision plate reader.

pcDNA3.1 expression vector encoding human truncated M2 receptor (residues 1-456; deletion of residues 233-374) vector has a cleavable hemagglutinin (HA) protein signal peptide derived from influenza virus (MKTIIALSYIFCLVFA; SEQ ID NO:3) followed by a FLAG-tag sequence (DYKDDDDA; SEQ ID NO:4) and is fused on the C-terminus via a flexible linker (GAQGNS-GSSGGGGSGGGGSSG; SEQ ID NO:5) to the large subunit of the NanoLuc luciferase (LgBit; SEQ ID NO:7). Nb9-8 (SEQ ID NO:1 in WO14/122183 and SEQ ID NO:25 herein) is fused via a flexible linker (GSSGGGGSGGGGSSG; SEQ ID NO:6) on the C-terminus to the small subunit of the NanoLuc luciferase (SmBiT; SEQ ID NO: 8). The ratio of DNA of the M2R expressing pcDNA3.1 vector and Nb9-8 expressing pcDNA3.1 vector during transfection was 1:10 (corresponds to 150 ng of the M2R expressing vector and 1.5 μg of the Nb9-8 expressing vector). Agonists Iperoxo (Sigma, SML0790), acetylcholine chloride (Tocris, 2809) and antagonists Atropine (Sigma, Y0000878), Tiotropium (Tocris, 5902) are applied at one or two concentrations in the assay. Samples and vehicle are prepared in Opti-MEM I reduced medium containing final 1% DMSO and 0.00022% Tween20.

Curve fitting and statistical analysis is performed in GraphPad Prism and data are represented as mean Area Under the Curve (AUC) and standard error on the mean. 2-3 replicates are implemented per data point. Data is represented as normalized AUC which corresponds to the ratio AUC(sample) over AUC(blank) and is normalized for potential well-to-well variations due to seeding.

The sequence of the M2 fusion used in this Example is given in Table 1 as SEQ ID NO:13. The sequence of the Nb9-8 fusion used in this Example is given in Table 1 as SEQ ID NO:17.

The results are shown in FIG. 11. As can be seen, using the assay of this example, it was possible to distinguish agonists from the antagonists and the reference (blank).

### Example 6: Screening Assay for Beta-2 Adrenergic Receptor

Same process as in Example 5 was used, except for that seeded cells were incubated for 30 minutes at 37° C., 5% CO2 before addition of Nano-Glo. pcDNA3.1 expression vector encoding human truncated (residues 2-365) Beta-2 adrenergic β2AR) vector has a cleavable hemagglutinin (HA) protein signal peptide derived from influenza virus (MKTIIALSYIFCLVFA; SEQ ID NO:3) followed by a FLAG-tag sequence (DYKDDDDA; SEQ ID NO:4) and is fused on the C-terminus via a flexible linker (GAQGNS-GSSGGGGSGGGGSSG; SEQ ID NO:5) to the large subunit of the NanoLuc luciferase (LgBit; SEQ ID NO:7). CA2780 (SEQ ID NO:4 in WO2012/007593 and SEQ ID NO:26 herein) is fused via a flexible linker (GSSGGGGSGGGGSSG; SEQ ID NO:6) on the C-terminus to the small subunit of the NanoLuc luciferase (SmBiT; SEQ ID NO:8). The ratio of DNA of the (32AR expressing pcDNA3.1 vector and CA2780 expressing pcDNA3.1 vector during transfection was 1:30 (corresponds to 50 ng of the (32AR expressing vector and 1.5 μg of the CA2780 expressing vector. Agonists isoprotenerol (Sigma, 15627), pindolol (Sigma, P0778), salmeterol (Sigma, S5068), adrenaline (Sigma, E4250), inverse agonist ICI 118,551 (Sigma, 1127) and antagonist carazolol (TCI Europe, C2578) are applied at a single concentration in the assay. Samples and vehicle are prepared in Opti-MEM I reduced medium containing final 1% DMSO.

The sequence of the Beta-2AR fusion used in this Example is given in Table 1 as SEQ ID NO:11. The sequence of the CA2780 fusion used in this Example is given in Table 1 as SEQ ID NO:16.

The results are shown in FIG. 12. As can be seen, using the assay of this example, it was possible to distinguish stronger agonists from weaker agonists and from the antagonist and the reference (blank). It was also possible to distinguish the inverse agonist from the antagonist and the reference (blank).

### Example 7: Screening Assay for Angiotensin II Receptor Type 1

Same process as in Example 5 was used, except for that seeded cells were incubated for 30 minutes at 37° C., 5% CO2 before addition of Nano-Glo. pcDNA3.1 expression vector encoding human truncated (residues 1-319) angiotensin II receptor type 1 (AT1R) vector has a cleavable hemagglutinin (HA) protein signal peptide derived from influenza virus (MKTIIALSYIFCLVFA; SEQ ID NO:3) followed by a FLAG-tag sequence (DYKDDDDA; SEQ ID NO:4) and is fused on the C-terminus via a flexible linker (GAQGNS-GSSGGGGSGGGGSSG; SEQ ID NO:5) to the large subunit of the NanoLuc luciferase (LgBit; SEQ ID NO:7). NbAT110i1 (SEQ ID NO:21) is fused via a flexible linker (GSSGGGGSGGGGSSG; SEQ ID NO:6) on the C-terminus to the small subunit of the NanoLuc luciferase (SmBiT; SEQ ID NO:8). The ratio of DNA of the AT1R expressing pcDNA3.1 vector and NbAT110i1 expressing pcDNA3.1 vector during transfection was 1:10 (corresponds to 37.5 ng AT1R expressing vector and 375 ng NbAT110i1 expressing vector). Agonists Angiotensin II (Tocris, 1562), [Sar1Ile8]-Angiotensin II (Santa Cruz Biotechnology, sc-391239) are applied at different concentrations in the assay and antagonists Candesartan (Tocris, 4791), Losartan (Chemscene LLC, CS-2116) are applied at one or two concentrations in the assay. Samples and vehicle are prepared in Opti-MEM I reduced medium containing final 1% DMSO and 0.00022% Tween20.

The sequence of the AT-1R fusion used in this Example is given in Table 1 as SEQ ID NO:14. The sequence of the NbAT110i1 fusion used in this Example is given in Table 1 as SEQ ID NO:18.

The results are shown in FIGS. 13 and 14. As can be seen, using the assay of this example, it was possible to establish dose response curves for the agonists used.

### Example 8: Screening of Compound Libraries

A library of 80 compounds (arrayed on a 96 well plate with 16 references) was screened using the assay essentially as described in Example 4. Cells were suspended in Opti-MEM and the compounds were added in Opti-MEM plus 0.0015% Tween. The cells were allowed to stabilize for 1 hour at room temperature after which NanoGlo was added (30 minutes at room temperature) followed by the compound to be tested (30 or 60 minutes).

The screening results are shown in FIG. 15. The obtained data for the reference compounds confirms that the assay can distinguish a known agonists from other references. The data for the 80 screened compounds shows that the screening assay is also capable of identifying hits from a library of compounds of unknown activity with respect to OX2R.

### Example 9: Screening Assay for Recombinant MCR4

Same process as in Example 4 was used. An expression vector encoding a recombinant MC4R receptor expression vector has a cleavable hemagglutinin (HA) protein signal peptide derived from influenza virus (MKTIIALSYIFCLVFA; SEQ ID NO:3) followed by a FLAG-tag sequence (DYKDDDDA; SEQ ID NO:4) and is fused on the C-terminus via a flexible linker (GAQGNS-GSSGGGGSGGGGSSG; SEQ ID NO:5) to the large subunit of the NanoLuc luciferase (LgBit; SEQ ID NO:7). CA2780 (SEQ ID NO:4 in WO 12/007593 and SEQ ID NO: 26 herein) is fused via a flexible linker (GSSGGGGSGGGGSSG; SEQ ID NO:6) on the C-terminus to the small subunit of the NanoLuc luciferase (SmBiT; SEQ ID NO:8). The ratio of DNA of the recombinant MC4R pBiT1.1C expressing vector and CA2780 pcDNA3.1 expressing vector during transfection was 1:1 (corresponds to 1.5 μg of each construct). Agonist NDP-alpha-MSH (Tocris, 3013), Rm-493 (Setmelanotide) (ChemScene LLC, CS-6399) and antagonist SHU9119 (Tocris, 3420) are applied at different concentrations in the assay. Samples and vehicle are prepared in Opti-MEM I reduced medium.

The sequence of the CA2780 fusion used in this Example is given in Table 1 as SEQ ID NO:16.

The results are shown in FIGS. 16 and 17. As can be seen, using the assay of this example, it was possible to distinguish agonists from the antagonists and the reference (blank) and to establish a dose response curve for one of the agonists.

### Example 10: Screening Assay for Recombinant OX2R

Same process as in Example 4 was used except for that a recombinant human OX2R receptor (SEQ ID NO:23) was expressed in pcDNA3.1 vector instead of pBiT1.1C. The recombinant OX2R expression vector has a cleavable hemagglutinin (HA) protein signal peptide derived from influenza virus (MKTIIALSYIFCLVFA; SEQ ID NO:3) followed by a FLAG-tag sequence (DYKDDDDK) and is fused on the C-terminus via a flexible linker (GAQGNS-GSSGGGGSGGGGSSG; SEQ ID NO:5) to the large subunit of the NanoLuc luciferase (LgBit; SEQ ID NO:7). XA8633 (SEQ ID NO:19 in WO14/118297 and SEQ ID NO:24 herein) is fused via a flexible linker (GSSGGGGSGGGGSSG; SEQ ID NO:6) on the C-terminus to the small subunit of the NanoLuc luciferase (SmBiT; SEQ ID NO:8). The ratio of DNA of the recombinant OX2R expressing vector and XA8633 expressing vector during transfection was 1:30 (corresponds to 50 ng of recombinant OX2R expressing vector and 1.5 μg XA8633 expressing vector). Agonists Orexin B (Tocris, 1456), TAK-925 (Enamine), CS-5456 (ChemScene LLC) and YNT-185 (Enamine) and antagonist EMPA (Tocris, 4558) are applied at different concentrations in the assay. Samples and vehicle are prepared in Opti-MEM I reduced medium containing final 1% DMSO and 0.0015% Tween20.

The sequence of the XA8633 fusion used in this Example is given in Table 1 as SEQ ID NO:15.

The results are shown in FIGS. 18 to 22. As can be seen, using the assay of this example, it was possible to distinguish agonists from the antagonists and to establish dose response curves for the agonists.

### Example 11: Screening Assay for Recombinant APJ Receptor

Same process as in Example 4 was used. pcDNA3.1 expression vector encoding a recombinant human APJ receptor has a cleavable hemagglutinin (HA) protein signal peptide derived from influenza virus (MKTIIALSYIFCLVFA; SEQ ID NO:3) followed by a FLAG-tag sequence (DYKDDDDA; SEQ ID NO:4) and is fused on the C-terminus via a flexible linker (GAQGNS-GSSGGGGSGGGGSSG; SEQ ID NO:5) to the large subunit of the NanoLuc luciferase (LgBit; SEQ ID NO:7). XA8633 (SEQ ID NO:24) is fused via a flexible linker (GSSGGGGSGGGGSSG; SEQ ID NO:6) on the C-terminus to the small subunit of the NanoLuc luciferase (SmBiT; SEQ ID NO:8). The ratio of DNA of the recombinant APJ receptor expressing pcDNA3.1 vector and XA8633 expressing pcDNA3.1 vector during transfection was 1:150 (corresponds to 10 ng of the recombinant APJ receptor expressing vector and 1.5 μg of the XA8633 expressing vector). Agonists [Pyr1]-Apelin-13 (Tocris, 2420), ELA-14 (Tocris, 6293), CMF-019 (Aobious, AOB8242) and antagonist MM 54 (Tocris, 5992) are applied at one or two concentrations in the assay. Samples and vehicle are prepared in Opti-MEM I reduced medium containing final 1% DMSO and 0.0015% Tween20.

The sequence of the recombinant APJ, which was an apelin/mu-opioid receptor chimer with the ECLs from apelin receptor and the ICLs from the mu-opioid receptor, is given as SEQ ID NO:22 and the sequence of the XA8633 fusion used in this Example is given in Table 1 as SEQ ID NO:15.

The results are shown in FIG. 23A. As can be seen, using the assay of this example, it was possible to distinguish strong agonists from weaker agonists and from the antagonist and the reference (blank).

In a separate experiment, instead of an apelin-mu-opiod receptor chimer, a apelin-beta-2AR receptor chimer with the ECLs from the apelin receptor and the ICLs from the beta-2AR receptor was used in an assay of the invention. The other fusion protein used was a CA2780-SmBiT fusion. The results are shown in FIG. 23B. In addition to the agonists [Pyr1]-Apelin-13 (Tocris, 2420), ELA-14 (Tocris, 6293), CMF-019 (Aobious, AOB8242) and antagonist MM-54 (Tocris, 5992), one further known APJ agonist (MM-07, Tocris, 7053) was tested. As can be seen, also when this other chimer was used in an assay of the invention, it was possible to distinguish strong agonists of the apelin receptor from weaker agonists and from the antagonist and the reference (blank), even if the assay window was not exactly the same as the assay window of the assay used in FIG. 23A.

### Example 12: Screening of Compound Libraries

A library of 80 compounds (arrayed on a 96 well plate with 16 references) was screened using the assay essentially described in Example 10. Cells were suspended in Opti-MEM and the compounds were added in Opti-MEM plus 0.0015% Tween. The cells were allowed to stabilize for 1 hour at room temperature after which NanoGlo was added (30 minutes at room temperature) followed by the compound to be tested (30 or 60 minutes).

The screening results are shown in FIGS. 24 (control plate) and 25 (screening plate). The data from the control plate confirms that the assay can distinguish a known agonist for OX2R (TAK925) from a reference (blank). The data for the screening plate shows that the screening assay is also capable of identifying hits from a library of compounds of unknown activity with respect to OX2R.

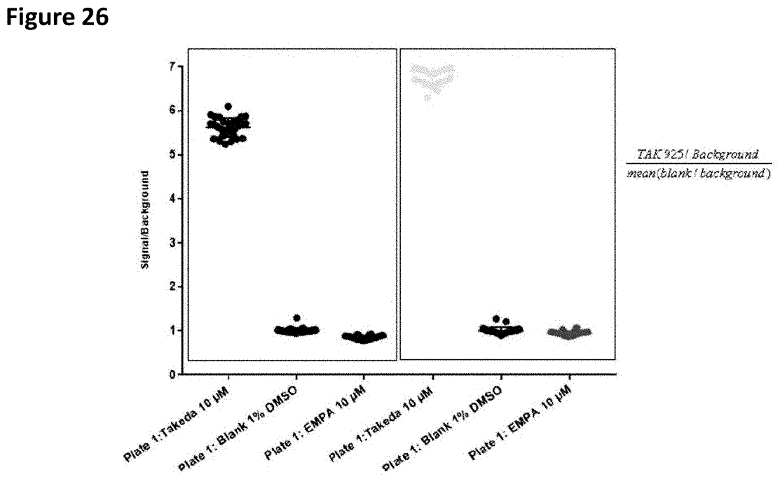

The same assay was used to perform a second screening run on a different library of 80 compounds (again, arrayed on a 96 well plate with 16 references) compounds. The results are shown in FIGS. 26 (control plate) and 27 (screening plate).

### Example 13: Comparison of Two Assay Formats of the Invention with a cAMP Assay (HTRF)

Three assay formats for testing compounds directed MC4R were compared: (i) a conventional homogeneous time resolved fluorescence (HTRF) cyclic AMP assay; (ii) an assay of the invention performed using a GPCR-LgBiT fusion (in which the GPCR was a recombinant GPCR essentially having the ECLs and TMs from MC4R and the ICLs for beta-2AR) and a CA2780-SmBiT fusion; and (iii) an assay of the invention performed using an MC4R-LgBiT fusion and a CA4435-35GS-CA4437-SmBiT fusion. Using these assays, the IC50 (for the cAMP HTRF assay) and EC50 values (assays of the invention) were determined for 5 compounds known to modulate MC4R as well as for a-MSH (reference). The results are listed in Table 2, with the two compounds that performed best in the cAMP assay also performing best in the assays of the invention and the compound that performed worse in the cAMP assay also performing worse in the assays of the invention.

The assays were performed as follows: The measurement of the accumulation of 3′,5′-cyclic adenosine monophosphate (cAMP) in intact CHO cells stably expressing human WT MC4R was performed using a LANCE® Ultra cAMP Kit (Perkin Elmer) according to manufacturer recommendations. Measurement of the signal was performed using Envision plate reader.

For the assays with MC4R-LgBiT fusion the same process as in Example 1 was used except for that a CA4435-35GS-CA4437-SmBiT fusion was used instead of CA4437-SmBiT fusion. The biparatopic CA4435-35GS-CA4437-SmBiT (linked by a 35GS linker, i.e. as CA4435-35GS-CA4437) is fused via a flexible linker (GSSGGGGSGGGGSSG; SEQ ID NO:6) on the N-terminus to the small subunit of the NanoLuc luciferase (SmBiT; SEQ ID NO:8). The ratio of DNA of the MC4R expressing pBiT1.1C vector and CA4435-35GS-CA4437 expressing pcDNA3.1 vector during transfection was 2:1 (corresponds to 0.5 μg of MC4R expressing vector and 0.25 μg of CA4435-35GS-CA4437 expressing vector).

For the assay with recombinant MC4R-LgBiT fusion the same process was used as in Example 9. pcDNA3.1 expression vector encoding recombinant MC4R has a cleavable hemagglutinin (HA) protein signal peptide derived from influenza virus (MKTIIALSYIFCLVFA; SEQ ID NO:3) followed by a FLAG-tag sequence (DYKDDDDA; SEQ ID NO:4) and is fused on the C-terminus via a flexible linker (GAQGNS-GSSGGGGSGGGGSSG; SEQ ID NO:5) to the large subunit of the NanoLuc luciferase (LgBit; SEQ ID NO:7). CA2780 (SEQ ID NO:4 in WO 12/007593 and SEQ ID NO: 26 herein) is fused via a flexible linker (GSSGGGGSGGGGSSG; SEQ ID NO:6) on the C-terminus to the small subunit of the NanoLuc luciferase (SmBiT; SEQ ID NO:8). The ratio of DNA of the recombinant MC4R pcDNA3.1 expressing vector and CA2780 pcDNA3.1 expressing vector during transfection was 1:100 (corresponds to 0.015 μg of recombinant MC4R expressing vector and 1.5 μg of CA2780 expressing vector).

Agonist alpha-MSH (Tocris, 2584) and 5 compounds known to modulate MC4R are applied at different concentrations in both assays. Samples and vehicle are prepared in Opti-MEM I reduced medium containing final 1% DMSO and 0.00022% Tween20. The cells were allowed to stabilize for 1 hour at room temperature after which NanoGlo was added (30 minutes at room temperature) followed by the compound to be tested (30 or 45 minutes). Luminescence is measured on Envision plate reader.

TABLE 2
Results from comparison of 3 assay formats

| | | Assay of the invention | |
|---|---|---|---|
| | cAMP | EC50 MC4R- | EC50 CA4435- |
| Compound | IC50 | B2AR | CA4437 biparatopic |
| 2-A | 8.00E−07 | 8.38E−09 | 7.90E−08 |
| 2-B | 5.44E−07 | 3.26E−09 | 5.79E−08 |
| a-MSH | 1.61E−07 | 1.48E−08 | 2.71E−08 |
| 2-C | 4.45E−06 | 5.46E−08 | 1.12E−06 |
| 2-D | 3.43E−06 | 8.30E−08 | 4.67E−07 |
| 2-E | 1.17E−05 | 1.32E−07 | 4.35E−06 |

### Example 14: Comparison of Two Assay Formats of the Invention

78 compound fragments were tested using two assays of the invention, both using a Beta-2AR-LgBiT fusion, but with one assay using an CA2780-SmBiT fusion and one assay using a CA4435-35GS-CA4437-SmBiT fusion, The results were plotted as a graph (FIG. 28), with each dot representing one compound and the x-axis representing the results obtained in the assay with the CA4435-35GS-CA4437-SmBiT fusion and the y-axis the results obtained in the assay with the CA2780-SmBiT. As can be seen from the resulting plot, there was a good correlation between the results obtained in each of the assays.

The assays were performed as follows: the assay with Beta-2AR-LgBiT fusion and CA2780-SmBiT fusion was essentially described in Example 6, except for that seeded cells were incubated for 60 minutes at 37° C., 5% CO2 before addition of Nano-Glo.

For the assay with Beta-2AR-LgBiT fusion and CA4435-35GS-CA4437-SmBiT fusion the same process as in Example 3 was used. The ratio of DNA of the (32AR expressing pBiT1.1C vector and CA4435-35GS-CA4437 expressing pcDNA3.1 vector during transfection was 2:1 (corresponds to 1 μg of β2AR expressing vector and 0.5 μg of CA4435-35GS-CA4437 expressing vector).

In both assays cells are suspended in Opti-MEM I reduced medium and the compounds are prepared in Opti-MEM containing final 0.00022% Tween20. Compounds are screened at 200 μM final concentration. The cells were allowed to stabilize for 1 hour at room temperature after which NanoGlo was added (30 minutes at room temperature) followed by the compound to be tested (30 minutes). Luminescence is measured on Spectramax i3x plate reader.

### Example 15: Comparison of Two Assay Formats of the Invention with a Radioligand Assay

The collection of compound fragments used in Example 14 was also tested using a conventional radioligand assay (see for example WO 2012/007593) and a corresponding assay of the invention. In FIG. 29A, the radioligand assay was performed using CA2780 and the assay of the invention was performed using a CA2780-SmBiT fusion. In FIG. 29B, the radioligand assay was performed using CA4435 and the assay of the invention was performed using a CA4435-35GS-CA4437-SmBiT fusion. In both FIG. 29A and FIG. 29B, the other fusion protein used in the assay of the invention was a beta-2AR-LgBiT fusion. The assays were essentially performed as described in Example 14.

The results were plotted in FIGS. 29A and 29B, respectively, with the x-axis representing the results obtained using the assay of the invention, the y-axis representing the results obtained with the radioligand assay in the assay, and each dot representing the result obtained for one of the compounds when tested in both the radioligand assay and the assay of the invention.

As can be seen, for both assays of the invention, overall the results obtained using an assay of the invention generally correlated with the results obtained using the corresponding radioligand assay.

### Example 16: Comparison of cAMP Assay and Assays of the Invention

A set of 14 compounds with confirmed activity against Beta-2AR was used in a GloSensor cAMP assay and in corresponding assays of the invention.

First, the activity of the compounds against Beta-2AR was determined/confirmed using a GloSensor cAMP assay, the results of which are shown in FIGS. 30A (compounds at 100 μM) and 30B (compounds at 200 μM). The controls used in the GloSensor assays were isoproterenol (“iso”, at 10 μM) in FIG. 30A and isoproterenol (“iso”, at 10 μM) and forskolin (“for”, also at 10 μM) in FIG. 30B.

The compounds were then tested in two corresponding assays of the invention (using a Beta-2AR-LgBiT fusion and a CA2780-SmBiT fusion or a CA4435-35GS-CA4437-SmBiT fusion, respectively). The results are given in Table 3 below.

As can be seen from the data in Table 3, for the 14 compounds used, there was overall good correlation between the results in the cAMP assay and the results obtained using the assays of the invention.

For both assays of invention the same process as in Example 14 was used.

The GloSensor cAMP assay (Promega) to monitor changes in the intracellular concentration of cAMP was performed according to the guidance from the manufacturer. For this assay HEK293T cells are transiently transfected with pcDNA3.1 expression vector encoding Beta-2AR-LgBiT fusion described in Example 3 and with pGloSensor™-22F plasmid at ratio 1:1. Compounds are applied at two concentrations (100 μM and 200 μM) and controls isoproterenol and forskolin at single concentration (10 μM). Samples and vehicle are prepared in Opti-MEM I reduced medium containing final 0.5% (for compounds at 100 μM) or 1% DMSO (for compounds at 200 μM). Luminescence is continuously monitored for 40 minutes (one measurement every 2 minutes) on Envision plate reader.

Curve fitting and statistical analysis is performed in GraphPad Prism and data are represented as mean Area Under the Curve (AUC) and standard error on the mean. 2 replicates are implemented per data point. Data is represented as Sum of AUC of double normalized data which corresponds to the ratio AUC(sample) over AUC(blank).

TABLE 3
comparison of assay formats using compounds with confirmed
activity against Beta-2AR.

| | Assay invention | | GloSensor |
|---|---|---|---|
| | | CA4435-35GS- | DN β2AR |
| Compound | CA2780 | CA4437 | (200 μM) |
| 3-A | 4.88 | 2.27 | 14.66 |
| 3-B | 3.20 | 1.61 | 8.43 |
| 3-C | 2.46 | 1.33 | 11.60 |
| 3-D | 1.63 | 1.27 | 2.99 |
| 3-E | 1.28 | 0.78 | 0.23 |
| 3-F | 1.72 | 1.21 | 4.74 |
| 3-G | 1.04 | 0.93 | 0.72 |
| 3-H | 1.59 | 1.14 | 2.46 |
| 3-I | 1.45 | 1.09 | 1.24 |
| 3-J | 1.35 | 1.17 | 1.29 |
| 3-K | 1.09 | 1.06 | 2.12 |
| 3-L | 1.08 | 1.03 | 1.02 |
| 3-M | 1.32 | 1.11 | 1.90 |
| 3-N | 1.00 | 1.01 | 0.64 |

### Example 17: Comparison of Cell-Based and Membrane-Based Assays of the Invention

A cell-based assay of the invention was compared to a corresponding membrane-based assay of the invention. The fusions used were a Beta-2AR-LgBiT fusion and a CA2780-SmBit fusion.

The cell-based assay of the invention was essentially performed as described in Example 6.

For the membrane-based assay, membrane extracts prepared from HEK293T cells expressing Beta-2AR-LgBiT fusion and purified CA2780-SmBiT prepared E. coli were used. Beta-2AR-LgBiT fusion membrane extracts were prepared from HEK293T cells by applying a homogenization protocol in presence of a Tris buffer supplemented with protease inhibitors using a Ultra-Turrax homogenizer.

8 μl of the Beta-2AR-LgBiT fusion membrane extracts and 8 μl of the CA2780-SmBiT purified protein diluted in Lumit™ Immunoassay dilution buffer (Promega) supplemented with 100 μM GTPγS were added to a white 384-well flat bottom non-binding plate. After 15 minutes incubation at room temperature, 8 μl of NanoGlo for Lumit Immunoassay (Promega) was added. NanoGlo was diluted 51 fold in Lumit Immunoassay dilution buffer supplemented with GTPγS prior to addition to the plate. After 30 minutes incubation at room temperature the background luminescence is read on SpectraMax i3x plate reader, after which 8 μl of compounds or vehicle prepared in Lumit Immunoassay buffer supplemented with GTPγS, 0.5% DMSO and 0.5% MilliQ were added and the luminescence was read for 30 minutes on plate reader.

The assay of the invention was performed using both the intact cells and the cell membrane, using isoproterenol (at 10 μM) and carazolol (also at 10 μM). The results are shown in FIGS. 31A (whole cells) and 31B (cell extract) and confirm that similar results can be obtained with whole cells and membrane extracts.

### Example 18. Use of an Assay of the Invention to Characterize ConfoBodies

As described herein, the methods and arrangements of the invention can not only be used in screens for identifying modulators of a translayer protein (e.g. agonists or antagonists binding to the extracellular binding site), but also to identify and/or characterize potential intracellular ligands of the receptor (and in particular, intracellular ligands that can stabilize a particular conformation of the translayer protein and/or stabilize/induce the formation of a complex between an extracellular ligand such as an agonist, the translayer protein and said intracellular ligand).

As a non-limiting example, it was investigated whether an assay of the invention can be used to characterize potential ConfoBodies.

In this example, a set of 11 VHH's resulting from screening/selection of a VHH library for possible VHHs binding to an intracellular epitope on the APJ receptor were characterized using an assay of the invention, to see if these VHH's could provide a dose-response curve when the APJ receptor in the assay was exposed to apelin. For this purpose, an assay of the invention was used in which the sequence of the apelin receptor was fused to LgBiT, and each of the VHH's was tested as a fusion to SmBiT.

The resulting DRCs are shown in FIG. 32A, and show that the assay of the invention could be used to confirm that the tested VHHs allow for a dose-dependent response to apelin to be generated in the assay of the invention, which confirms that these VHHs are capable of inducing/stabilizing the formation of an APJ/APJ-receptor∧VHH complex. These findings are confirmed in FIGS. 32B and C, which show DRCs that were generated for 4 of these 11 VHHs in response to both APJ (FIG. 32B) and CMF-19 (FIG. 32C).

The assay was essentially performed as described in Example 11. The ratio of the DNA of the wild type human APJ receptor expressing pcDNA3.1 vector and each of the VHH expressing pcDNA3.1 vectors during transfection was 1:30.

### Example 19: Identification of Positive Allosteric Modulators

This example shows that an assay of the invention can be used to identify and/or characterize a positive allosteric modulators. To demonstrate this, the influence of LY2119620 (a known positive modulator of the M2 receptor, Croy et al., Mol Pharmacol. 2014 July; 86(1):106-15) on the dose response curve of iperoxo (a known M2 superagonist) was tested using an assay of the invention (M2-LgBiT fusion and an Nb9-8-SmBiT fusion).

As can be seen from the resulting DRC (FIG. 33), using the assay of the invention, it was possible to detect the effect as a positive allosteric modulator that LY2119620 has on the dose-response curve of iperoxo. Furthermore, using the same assay of the invention, it was also possible to show that LY2119620 itself has some agonistic effect on the M2 receptor (data not shown).

### Example 20: Screening of Compound Library

A plate of small chemical compounds (fragment library) was screened in an assay of the invention using a recombinant OX2R-MOR chimer (SEQ ID NO:23) fused to LgBiT and XA8633 fused to SmBiT (see Example 10) and in a commercially available OX2 IP-One assay.

The results are plotted in FIG. 34, with the x-axis representing the data obtained in the assay of the invention, the y-axis representing the data obtained in the IP-One assay and each dot representing the results for a single compound. As can be seen, there was a reasonable degree of correlation between the results obtained using the assay of the invention and the results obtained in the IP-One assay. A similar degree of correlation was observed when the results obtained using the same assay of the invention were compared to the results obtained in an OX2 radioligand assay (data not shown).

### Example 21: Screening of Large Compound Library

A library of 11378 compounds was screened in an assay of the invention using a recombinant OX2R-MOR chimer (SEQ ID NO:23) fused to LgBiT and XA8633 fused to SmBiT (see Example 10).

The results are plotted in FIGS. 35A (compounds tested at 3004) and 35B (compounds tested at 200 μM), with the x-axis representing the ratio of the signal obtained with the compound tested (“sample”) vs signal given by the carrier solvent (“blank”) and each dot representing the result obtained for a single compound.

As can be seen from these plots, screening of the large compound library using the assay of the invention afforded multiple hits.

1. Arrangement that comprises at least the following elements:
a boundary layer that separates a first environment from a second environment;
a translayer protein;
a first ligand for the translayer protein that is present in the first environment;
a second ligand for the translayer protein that is present in the second environment; and
a binding pair that consists of at least a first binding member and a second binding member, which binding pair is capable of generating a detectable signal.

2. Arrangement according to claim 1, in which the first binding member of the binding pair is part of a first fusion protein comprising the first binding member that is fused or linked, either directly or via a suitable linker or spacer, to the translayer protein.

3. Arrangement according to claim 1, in which the second ligand is a protein, ligand, binding domain, binding unit or other chemical entity that specifically binds to one or more functional, active and/or druggable conformations of the translayer protein, that induces the formation of and/or stabilizes one or more functional, active and/or druggable conformations of the translayer protein (and/or shifts the conformational equilibrium of the translayer protein towards one or more such conformations); and/or that induces the formation of and/or stabilizes a complex of the translayer protein, the first ligand and the second ligand.

4. Arrangement according to claim 1, in which the second ligand is part of a (second) fusion protein comprising the second binding member of the binding pair that is fused or linked, either directly or via a suitable linker or spacer, to the second ligand.

5. Arrangement according to claim 1, in which the second ligand is an immunoglobulin single variable domain.

6. Arrangement according to claim 1, in which the second ligand is a naturally occurring ligand of the translayer protein or an analog, derivative or ortholog of such a naturally occurring ligand.

7. Arrangement according to claim 1, in which the second member of the binding pair is part of a (second) fusion protein comprising the second binding member of the binding pair that is fused or linked, either directly or via a suitable linker or spacer, to a binding domain or binding unit that can bind to the second ligand.

8. Arrangement according to claim 7, in which the binding domain or binding unit is an immunoglobulin single variable domain.

9. Arrangement according to claim 1, in which the second ligand is part of a protein complex that comprises the second ligand and one or more further proteins, which protein complex binds to, or is bound to, the translayer protein.

10. Arrangement according to claim 1, in which the second member of the binding pair is part of a (second) fusion protein comprising the second binding member of the binding pair that is fused or linked, either directly or via a suitable linker or spacer, to a binding domain or binding unit that can bind to the second ligand.

11. Arrangement according to claim 10, in which the binding domain or binding unit is an immunoglobulin single variable domain.

12. Arrangement according to claim 1, in which the second binding member of the binding pair is part of a second fusion protein comprising the second binding member of the binding pair that is fused or linked, either directly or via a suitable linker or spacer, to a protein that can bind directly or indirectly to the translayer protein.

13. Arrangement according to claim 1, in which the second binding member of the binding pair is part of a second fusion protein comprising the second binding member of the binding pair that is fused or linked, either directly or via a suitable linker or spacer, to a protein that can bind directly to the translayer protein (said protein being the second ligand).

14. Arrangement according to any of claim 1, in which the second binding member of the binding pair is part of a second fusion protein comprising the second binding member of the binding pair that is fused or linked, either directly or via a suitable linker or spacer, to a protein that can bind directly to the translayer protein (said protein being the second ligand), in which the protein that can bind directly to the translayer protein is a protein, ligand, binding domain, binding unit or other chemical entity that specifically binds to one or more functional, active and/or druggable conformations of the translayer protein, that induces the formation of and/or stabilizes one or more functional, active and/or druggable conformations of the translayer protein (and/or shifts the conformational equilibrium of the translayer protein towards one or more such conformations); and/or that induces the formation of and/or stabilizes a complex of the translayer protein, the first ligand and the second ligand.

15. Arrangement according to claim 13, in which the protein that can bind directly to the translayer protein is an immunoglobulin single variable domain.

16. Arrangement according to claim 1, in which the second binding member of the binding pair is part of a second fusion protein comprising the second binding member of the binding pair that is fused or linked, either directly or via a suitable linker or spacer, to a protein that can bind indirectly to the translayer protein.

17. Arrangement according to claim 16, in which the second binding member of the binding pair is part of a second fusion protein comprising the second binding member of the binding pair that is fused or linked, either directly or via a suitable linker or spacer, to a binding domain or binding unit protein can bind to the second ligand.

18. Arrangement according to claim 17, in which the second binding member of the binding pair is part of a second fusion protein comprising the second binding member of the binding pair that is fused or linked, either directly or via a suitable linker or spacer, to a binding domain or binding unit protein can bind to the second ligand, in which the second ligand is a protein, ligand, binding domain, binding unit or other chemical entity that specifically binds to one or more functional, active and/or druggable conformations of the translayer protein, that induces the formation of and/or stabilizes one or more functional, active and/or druggable conformations of the translayer protein (and/or shifts the conformational equilibrium of the translayer protein towards one or more such conformations); and/or that induces the formation of and/or stabilizes a complex of the translayer protein, the first ligand and the second ligand.

19. Arrangement according to claim 17, in which the protein binding domain or binding unit protein can bind to the second ligand is an immunoglobulin single variable domain.

20. Arrangement according to claim 16, in which the second binding member of the binding pair is part of a second fusion protein comprising the second binding member of the binding pair that is fused or linked, either directly or via a suitable linker or spacer, to a binding domain or binding unit protein can bind to a protein complex that comprises the second ligand, which protein complex is bound to, or can bind to, the translayer protein.

21. Arrangement according to claim 20, in which the protein binding domain or binding unit protein can bind to the protein complex is an immunoglobulin single variable domain.

22. Arrangement that comprises at least the following elements:
a boundary layer that separates a first environment from a second environment;
a translayer protein;
a ligand for the translayer protein that is present in the second environment; and
a binding pair that consists of at least a first binding member and a second binding member, which binding pair is capable of generating a detectable signal.

23. Arrangement according to claim 22, in which the boundary layer is a cell wall or cell membrane.

24. Arrangement according to claim 22, in which the boundary layer is the wall or membrane of a liposome or vesicle.

25. Arrangement according to claim 22, in which the translayer protein is a GPCR.

26. Method comprising the steps of:
a) providing an arrangement according to claim 22; and
b) adding a first ligand to the first environment of said arrangement.

27. Method according to claim 26, further comprising the step of:
c) measuring the signal that is generated by the binding pair and/or measuring the change in the signal that is generated by the binding pair.


SEQUENCE LISTING (26 sequences)

SEQ ID NO: 1 - protein, 128 aa, Artificial sequence
  CA4435
QVQLQESGGGLVQPGGSLRLSCAASGFTFSNYKMNWVRQAPGKGLEWVSDISQSGASISYTGSVKGRFTISRDNAKNTLYLQMNSLKPEDTAVYYCARCPAPFTRDCFDVTSTTYAYRGQGTQVTVSS

SEQ ID NO: 2 - protein, 119 aa, Artificial sequence
  CA4437
QVQLQESGGGFVQAGGSLRLSCAASGSIFSKNTMAWFRQAPGKERELVAASPTGGSTAYKDSVKGRFTISRDSAKNTVLLQMNVLKPEDTAVYYCHLRQNNRGSWFHYWGQGTQVTVSS

SEQ ID NO: 3 - protein, 16 aa, Artificial sequence
  Hemagglutinin (HA) protein signal peptide
MKTIIALSYIFCLVFA

SEQ ID NO: 4 - protein, 8 aa, Artificial sequence
  FLAG-tag
DYKDDDDA

SEQ ID NO: 5 - protein, 21 aa, Artificial sequence
  Linker
GAQGNSGSSGGGGSGGGGSSG

SEQ ID NO: 6 - protein, 15 aa, Artificial sequence
  Linker
GSSGGGGSGGGGSSG

SEQ ID NO: 7 - protein, 158 aa, Artificial sequence
  large subunit of the NanoLuc luciferase (LgBit)
VFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 8 - protein, 11 aa, Artificial sequence
  small subunit of the NanoLuc luciferase (SmBit)
VTGYRLFEEIL

SEQ ID NO: 9 - protein, 535 aa, Artificial sequence
  MC4R fusion
MKTIIALSYIFCLVFADYKDDDDKAVNSTHRGMHTSLHLWNRSSYRLHSNASESLGKGYSDGGCYEQLFVSPEVFVTLGVISLLENILVIVAIAKNKNLHSPMYFFICSLAVADMLVSVSNGSETIVITLLNSTDTDAQSFTVNIDNVIDSVICSSLLASICSLLSIAVDRYFTIFYALQYHNIMTVKRVGIIISCIWAACTVSGILFIIYSDSSAVIICLITMFFTMLALMASLYVHMFLMARLHIKRIAVLPGTGAIRQGANMKGAITLTILIGVFVVCWAPFFLHLIFYISCPQNPYCVCFMSHFNLYLILIMCNSIIDPLIYALRSQELRKTFKEIICCYPLGGLCDLSSRYGAQGNSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 10 - protein, 666 aa, Artificial sequence
  GLP-1R fusion
MKTIIALSYIFCLVFADYKDDDDAMAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCSGAQGNSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 11 - protein, 575 aa, Artificial sequence
  Beta-2AR fusion
MKTIIALSYIFCLVFADYKDDDDAENLYFQGFGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYAEETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGGAQGNSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 12 - protein, 559 aa, Artificial sequence
  MOR fusion
MKTIIALSYIFCLVFADYKDDDDAMAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIGAQGNSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 13 - protein, 517 aa, Artificial sequence
  M2 fusion
MKTIIALSYIFCLVFADYKDDDDAMNNSTNSSNNSLALTSPYKTFEVVFIVLVAGSLSLVTIIGNILVMVSIKVNRHLQTVNNYFLFSLACADLIIGVFSMNLYTLYTVIGYWPLGPVVCDLWLALDYVVSNASVMNLLIISFDRYFCVTKPLTYPVKRTTKMAGMMIAAAWVLSFILWAPAILFWQFIVGVRTVEDGECYIQFFSNAAVTFGTAIAAFYLPVIIMTVLYWHISRASKSRIKKDKKEPVANQDPVSKKPPPSREKKVTRTILAILLAFIITWAPYNVMVLINTFCAPCIPNTVWTIGYWLCYINSTINPACYALCNATFKKTFKHLLMGAQGNSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 14 - protein, 522 aa, Artificial sequence
  AT1R fusion
MKTIIALSYIFCLVFADYKDDDDAMILNSSTEDGIKRIQDDCPKAGRHNYIFVMIPTLYSIIFVVGIFGNSLVVIVIYFYMKLKTVASVFLLNLALADLCFLLTLPLWAVYTAMEYRWPFGNYLCKIASASVSFNLYASVFLLTCLSIDRYLAIVHPMKSRLRRTMLVAKVTCIIIWLLAGLASLPAIIHRNVFFIENTNITVCAFHYESQNSTLPIGLGLTKNILGFLFPFLIILTSYTLIWKALKKAYEIQKNKPRNDDIFKIIMAIVLFFFFSWIPHQIFTFLDVLIQLGIIRDCRIADIVDTAMPITICIAYFNNCLNPLFYGFLGKKFKRYFLQLLKYGAQGNSGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 15 - protein, 149 aa, Artificial sequence
  XA8633 fusion
MQVQLQESGGGLVRPGGSRRLSCVDSERTSYPMGWFRRAPGKEREFVASITWSGIDPTYADSVADRFTISRDVANNTLYLQMNSLKHEDTAVYYCAARAPVGQSSSPYDYDYWGQGTQVTVSSGSSGGGGSGGGGSSGVTGYRLFEEIL

SEQ ID NO: 16 - protein, 147 aa, Artificial sequence
  CA2780 fusion
MQVQLQESGGGLVQAGGSLRLSCAASGSIFSINTMGWYRQAPGKQRELVAAIHSGGSTNYANSVKGRFTISRDNAANTVYLQMNSLKPEDTAVYYCNVKDYGAVLYEYDYWGQGTQVTVSSGSSGGGGSGGGGSSGVTGYRLFEEIL

SEQ ID NO: 17 - protein, 148 aa, Artificial sequence
  Nb9-8 fusion
MQVQLVESGGGLVQAGDSLRLSCAASGFDFDNFDDYAIGWFRQAPGQEREGVSCIDPSDGSTIYADSAKGRFTISSDNAENTVYLQMNSLKPEDTAVYVCSAWTLFHSDEYWGQGTQVTVSSGSSGGGGSGGGGSSGVTGYRLFEEIL

SEQ ID NO: 18 - protein, 153 aa, Artificial sequence
  NbAT110i1 fusion
MQVQLQESGGGLVQAGGSLRLSCAASGNIFDVDIMGWYRQAPGKERELVASITDGGSTDYADSVKGRFTISRDNAKNTVYLQMNSLKPEDTAVYYCAAVAYPDIPTYFDYDSDNFYWGQGTQVTVSSGSSGGGGSGGGGSSGVTGYRLFEEIL

SEQ ID NO: 19 - protein, 146 aa, Artificial sequence
  CA4437 fusion
MQVQLQESGGGFVQAGGSLRLSCAASGSIFSKNTMAWFRQAPGKERELVAASPTGGSTAYKDSVKGRFTISRDSAKNTVLLQMNVLKPEDTAVYYCHLRQNNRGSWFHYWGQGTQVTVSSGSSGGGGSGGGGSSGVTGYRLFEEIL

SEQ ID NO: 20 - protein, 155 aa, Artificial sequence
  CA4435 fusion
MQVQLQESGGGLVQPGGSLRLSCAASGFTFSNYKMNWVRQAPGKGLEWVSDISQSGASISYTGSVKGRFTISRDNAKNTLYLQMNSLKPEDTAVYYCARCPAPFTRDCFDVTSTTYAYRGQGTQVTVSSGSSGGGGSGGGGSSGVTGYRLFEEIL

SEQ ID NO: 21 - protein, 127 aa, Artificial sequence
  NbAT110i1
MQVQLQESGGGLVQAGGSLRLSCAASGNIFDVDIMGWYRQAPGKERELVASITDGGSTDYADSVKGRFTISRDNAKNTVYLQMNSLKPEDTAVYYCAAVAYPDIPTYFDYDSDNFYWGQGTQVTVSS

SEQ ID NO: 22 - protein, 356 aa, Artificial sequence
  APL receptor-MOR chimer
MKTIIALSYIFCLVFADYKDDDDAMEEGGDFDNYYGADNQSECEYTDWKSSGALIPAIYMLVFLLGTTGNGLVLWTIVRYTKMKRRSADIFIASLAVADLTFVVTLPLWATYTYRDYDWPFGTFFCKLSSYLIFVNMYASVFCLTGLSFDRYLAICHPVKALDFRTPRNGAVATAVLWVLAALLAMPVMVLRTTGDLENTTKVQCYMDYSMVATVSSEWAWEVGLGVSSTTVGFVVPFTIMLTCYGLMILRLKSVRMLSGSKEKDRNLRRILSIIVVLVVTFALCWMPYHLVKTLYMLGSLLHWPCDFDLFLMNIFPYCTCISYVNSCLNPFLYAFLDENFKRCFREFCIPTSSNI

SEQ ID NO: 23 - protein, 355 aa, Artificial sequence
  OX2-MOR chimer
MSGTKLEDSPPCRNWSSASELNETQEPFLNPTDYDDEEFLRYLWREYLHPKEYEWVLIAGYIIVFVVALIGNVLVMYVIVRYTKMKTATNYFIVNLSLADVLVTITCLPATLVVDITETWFFGQSLCKVIPYLQTVSVSVSVLTLSCIALDRYIAVCHPVKALDFRTPRNARNSIVIIWIVSCIIMIPQAIVMECSTVFPGLANKTTLFTVCDERWGGEIYPKMYHICFFLVTYMAPLCLMVLAYGLMILRLKSVRMLSGSKEKDRARRKTARMLMIVLLVFAICYLPISILNVLKRVFGMFAHTEDRETVYAWFTFSHWLVYANSAANPIIYAFLDENFKRCFREFCIPTSSNI

SEQ ID NO: 24 - protein, 122 aa, Artificial sequence
  XA8633
QVQLVESGGGLVRPGGSRRLSCVDSERTSYPMGWFRRAPGKEREFVASITWSGIDPTYADSVADRFTISRDVANNTLYLQMNSLKHEDTAVYYCAARAPVGQSSSPYDYDYWGQGTQVTVSS

SEQ ID NO: 25 - protein, 121 aa, Artificial sequence
  Nb 9-8
QVQLQESGGGLVQAGDSLRLSCAASGFDFDNFDDYAIGWFRQAPGQEREGVSCIDPSDGSTIYADSAKGRFTISSDNAENTVYLQMNSLKPEDTAVYVCSAWTLFHSDEYWGQGTQVTVSS

SEQ ID NO: 26 - protein, 120 aa, Artificial sequence
  CA2780
QVQLQESGGGLVQAGGSLRLSCAASGSIFSINTMGWYRQAPGKQRELVAAIHSGGSTNYANSVKGRFTISRDNAANTVYLQMNSLKPEDTAVYYCNVKDYGAVLYEYDYWGQGTQVTVSS